(19) United States
(12) Patent Application Publication
(10) Pub. No.: US 2026/0250665 A1
(43) Pub. Date: Aug. 27, 2026

(54) SYSTEMS AND METHODS FOR SEQUENCE DISPLAY-ENABLED MACHINE LEARNING: UNLOCKING LARGE-SCALE DATA FOR PROTEIN EVOLUTION

(71) Applicants: William Marsh Rice University, Houston, TX (US); The Johns Hopkins University, Baltimore, MD (US)

(72) Inventors: Xiongyi Huang, Ellicott City, MD (US); Jinyan Rui, Baltimore, MD (US); Han Xiao, Houston, TX (US); Linqi Cheng, Houston, TX (US)

(21) Appl. No.: 19/550,066

(22) Filed: Feb. 25, 2026

Related U.S. Application Data
(60) Provisional application No. 63/763,079, filed on Feb. 25, 2025.

(51) Int. Cl. C12N 15/10; C12N 9/22; C12N 9/78; C12N 15/11; G16B 30/00

(52) U.S. Cl. CPC C12N 15/1065; C12N 9/226; C12N 9/78; C12N 15/11; G16B 30/00; C07K 2319/00; C12N 2310/20

(57) ABSTRACT

Disclosed herein are systems, methods, compositions, and kits for generating sequence-activity datasets for variants of a protein of interest (POI).

### CROSS-REFERENCE TO RELATED APPLICATIONS

This application claims priority from U.S. provisional application 63/763,079, filed Feb. 25, 2025, the entire contents of which are herein incorporated by reference.

### REFERENCE TO A SEQUENCE LISTING

The application contains a Sequence Listing which has been submitted electronically in .xml format and is hereby incorporated by reference in its entirety. Said .xml copy, created on Feb. 24, 2026, is named “130492-878145_SEQLISTING.xml” and is 24,576 bytes in size.

### FIELD

The present disclosure generally relates to protein evolution analysis, and in particular, to a system and associated method for generating of large-scale protein sequence-activity datasets.

### BACKGROUND

Engineering proteins with desired functions remains a significant challenge, often hindered by the inefficiency of traditional methods and the lack of comprehensive sequence-activity datasets.

It is with these observations in mind, among others, that various aspects of the present disclosure were conceived and developed.

### BRIEF SUMMARY

In some aspects, the techniques described herein relate to a system including: (a) a plasmid library including a plurality of plasmids, each plasmid including: (i) a nucleic acid sequence encoding a variant of a protein of interest (POI); and (ii) a recording barcode including a nucleic acid sequence positioned in cis with the nucleic acid sequence encoding the variant of the POI; (b) a writer enzyme including a base-editing enzyme capable of introducing one or more sequence modifications in the recording barcode, wherein activity of the variant of the POI modulates expression or activity of the base-editing enzyme such that sequence modifications are introduced into the recording barcode in an activity-dependent manner; and (c) a sequencing and data-processing subsystem including: (i) a sequencing module configured to determine sequences of the nucleic acid sequence encoding the variant of the POI and the corresponding recording barcode; and (ii) at least one processor in communication with a memory storing instructions that, when executed, cause the processor to correlate sequence information of the variant of the POI with sequence modifications in the corresponding recording barcode to generate a sequence-activity dataset.

In some aspects, the techniques described herein relate to a method of generating a sequence-activity dataset for variants of a protein of interest (POI), the method including: (a) constructing a plasmid library including a plurality of plasmids, each plasmid including: (i) a nucleic acid sequence encoding a variant of the POI; and (ii) a recording barcode including a nucleic acid sequence positioned in cis with the nucleic acid sequence encoding the variant of the POI; (b) expressing the plasmid library in a cell in the presence of a writer enzyme capable of introducing one or more sequence modifications in the recording barcode; (c) coupling biological activity of each variant of the POI to expression or activity of the writer enzyme such that sequence modifications are introduced into the corresponding recording barcode in an activity-dependent manner; (d) sequencing the nucleic acid sequence encoding the variant of the POI and the corresponding recording barcode; and (e) using at least one processor in communication with a memory to correlate the nucleic acid sequence encoding the variant of the POI with sequence modifications in the corresponding recording barcode to generate a sequence-activity dataset.

In some aspects, the techniques described herein relate to a method of recording biological activity of protein variants in nucleic acid memory, the method including: (a) providing a plurality of nucleic acid constructs, each construct including: (i) a nucleic acid sequence encoding a variant of a protein of interest (POI); and (ii) a recording sequence including one or more editable nucleotides; (b) functionally coupling biological activity of each variant of the POI to activity of a programmable writer enzyme such that the writer enzyme modifies the recording sequence in proportion to or in response to the biological activity of the variant; (c) permitting the writer enzyme to introduce one or more sequence modifications into the recording sequence; (d) determining the sequence of the nucleic acid sequence encoding the variant of the POI and the sequence of the modified recording sequence; and (e) correlating, using at least one processor, the sequence of the variant of the POI with the sequence modifications in the recording sequence to generate a dataset associating protein sequence with recorded biological activity.

In some aspects, the techniques described herein relate to a plasmid library including a plurality of plasmids, each plasmid including: (a) a nucleic acid sequence encoding a variant of a protein of interest (POI); and (b) a recording barcode including a nucleic acid sequence positioned on the same plasmid as the nucleic acid sequence encoding the variant of the POI.

In some aspects, the techniques described herein relate to an isolated nucleic acid including: (a) a nucleic acid sequence encoding a variant of a protein of interest (POI); and (b) a recording sequence including one or more editable nucleotides positioned in cis with the nucleic acid sequence encoding the variant of the POI.

In some aspects, the techniques described herein relate to a kit for generating a sequence-activity dataset for variants of a protein of interest (POI), the kit including: (a) a plasmid library including a plurality of plasmids, each plasmid including: (i) a nucleic acid sequence encoding a variant of the POI; and (ii) a recording barcode including one or more editable nucleotides positioned on the same plasmid as the nucleic acid sequence encoding the variant of the POI; (b) a nucleic acid construct encoding a programmable writer enzyme capable of introducing sequence modifications into the recording barcode; (c) one or more reagents for expressing the plasmid library and the programmable writer enzyme in a host cell; and (d) instructions for: (i) coupling biological activity of variants of the POI to activity of the programmable writer enzyme to generate activity-dependent sequence modifications in the recording barcode; (ii) sequencing the nucleic acid sequence encoding the variants of the POI and the corresponding recording barcodes; and (iii) correlating variant sequences with barcode modifications to generate a sequence-activity dataset.

### BRIEF DESCRIPTION OF THE DRAWINGS

The present patent or application file contains at least one drawing executed in color. Copies of this patent or patent application publication with color drawing(s) will be provided by the Office upon request and payment of the necessary fee.

FIGS. 1A-1C are a series of diagrams showing a pipeline of a Sequence Display-Enabled Machine Learning (SDEML) system outlined herein. (A) Sequence Display platform for generating large-scale sequence-activity datasets. (B) Machine learning pipeline leveraging pre-trained pLMs for protein evolution using data generated by Sequence Display. (C) Ensemble strategy for activity inference and evaluation of protein variants.

FIG. 2 is a diagram showing a general design scheme of Sequence Display.

FIG. 3 is a diagram showing design of a recording sequence and the connection between UGI domain mutations and editing activity of a cytosine base editor.

FIG. 4 is a graphical representation showing initial data collection and machine learning model development.

FIG. 5 is a simplified diagram showing design of a genetic circuit that link the activity of RNase III with the activity of base editor.

FIGS. 6A-6D are a series of graphical representations showing SlugCas9 evolution towards diverse PAMs using SDEML. (A) Pipeline of SlugCas9 evolution targeting diverse PAMs using SDEML. (B) docking structure of SlugCas9-WT with the recording barcode and NNGG PAM. (C) Evaluation of the accuracy of SD evolution system using SlugCas9-WT and various variants. (D) Evaluation of the accuracy of SD evolution system using the variants from 2NNK SlugCas9 library.

FIGS. 7A-7C are a series of graphical representations showing 5NNK SlugCas9 library for sequence display evolution. (a) Identification of SlugCas9 variants with general PAM recognition and specificity for three distinct PAMs from large-scale NGS data. (b) Comparison of PAM activities from large-scale sequence data and NGS validation for SlugCas9-WT and the top-performing variant, SlugCas9-GRRTR. (C) Evaluation of the accuracy of SD evolution system using the variants from 5NNK SlugCas9 library.

FIGS. 8A-8E are a series of graphical representations modeling the relationship between SlugCas9 variants and their activities using different models. (a) Seven evaluation metrics used to compare the performance of different fine-tuning strategies for pLMs. ESM-2 W/O FT: ESM-2 without fine-tuning; ESM-2 FT11L: ESM-2 fine-tuned on the last layer (11th layer); ESM-2 FT10-11 L: ESM-2 fine-tuned on the last two layers (10th and 11th layers); SP FT11L: Saprot fine-tuned on the last layer (11th layer); SP FT10-11L: Saprot fine-tuned on the last two layers (10th and 11th layers). (b) Scatter plots of test datasets for four PAMs modeled using 5NNK SlugCas9 variants with ESM-2 fine-tuned on the last layer. (c) Scatter plots of test datasets for four PAMs modeled using 5NNK SlugCas9 variants with Saprot fine-tuned on the last layer. (d) Seven evaluation metrics used to compare the performance of different models. (e) Seven evaluation metrics used to compare the performance of 5-fold ensemble models for ESM-2 and Saprot.

FIGS. 9A and 9B are a series of graphical representations showing NGS validation of SlugCas9 hits predicted by ensemble models. (a) NGS validation of the top 25 SlugCas9 variants ranked by average mutation numbers. (b) NGS validation of the top 22 SlugCas9 variants ranked by average rank.

FIG. 10 is a simplified diagram showing an example computing system for implementation of aspects of the SDEML system of FIGS. 1A-5.

FIGS. 11A-11D are a series of simplified illustrations showing examples of a writing plasmid and a corresponding recording plasmid.

FIG. 12A is a graphical representation of the function and mechanism of UGI. UDG initiates base excision repair by removing uracil from dsDNA, creating an abasic site that prevents successful base editing. UGI inhibits UDG activity, preserving uracil in dsDNA and allowing mismatch repair to facilitate successful base editing.

FIG. 12B is a graphical depiction of the structure and binding pocket of UGI in complex with EcUDG. Leu58 and Tyr65 in UGI interact with Leu191 and Ala133 in EcUDG (PDB: 2UUG). The zoom-in interface view highlights the hydrophobic interaction between UGI Leu58 and EcUDG Leu191 and the hydrogen bonding between UGI Tyr65 and EcUDG Ala133.

FIG. 12C is a graphical depiction of the plasmid design for a Sequence Display platform for protein evolution comprising a recording plasmid comprising a promoter, rAPOBEC1, dSlugCas9, and a recording barcode, and a targeting plasmid comprising a promoter, a sgRNA and target DNA.

FIG. 12D is a graphical depiction of the plasmid design for evaluating the function of the degron tag. The sfGFP gene is fused to an N-terminal 6×His tag and a C-terminal degron tag. The N-terminal 6×His tag allows detection by western blot using an Anti-6×His Antibody-HRP. A construct lacking the C-terminal degron tag fused to sfGFP serves as a positive control.

FIG. 12E is a bar graph showing fluorescence-based evaluation of the degron tag effectiveness. Constructs encoding 6×His-sfGFP-degron and 6×His-sfGFP (control) were expressed in parallel, and their fluorescence signals were quantified using a plate reader following overnight expression.

FIG. 12F is a graphical depiction of the plasmid design for evaluating degron tag effectiveness in the base editor system. In the experimental group, one plasmid encodes sfGFP fused to a C-terminal degron tag via a Trp codon-containing linker (20 bp target sequence), along with a sgRNA designed to guide the base editor complex to this target sequence. A second plasmid expresses the rAPOBEC1-dSlugCas9-UGI base editor, which introduces a C-to-T conversion within the 20 bp target DNA, converting the Trp codon into a stop codon and thus removing the degron tag from sfGFP. For the negative control, a mismatched sgRNA prevents base editing and degron removal, leaving sfGFP fused to the degron tag. For the positive control, sfGFP is expressed without a degron tag.

FIG. 12G is a bar graph showing fluorescence analysis of degron tag effectiveness in the base editor system. The positive control, expressing sfGFP without a degron tag, showed a strong fluorescence signal. The experimental group (sfGFP-degron+base editor system) exhibited an intermediate fluorescence signal due to the removal of the degron tag via C-to-T editing by the rAPOBEC1-dSlugCas9-UGI complex. The negative control, expressing sfGFP fused with the degron tag (no editing), displayed a weak fluorescence signal. Statistical significance is indicated as P<0.05 (*), P<0.01 (**), P<0.001 (***), P<0.0001 (****) and ns (not significant).

FIG. 12H is a graphical depiction of the Degron-based fluorescence assay for validating the activity of UGI variants. The recording plasmid contains sgRNA and sfGFP with a degron tag, while the editing plasmid encodes rAPOBEC1, and UGI variants. sfGFP is fused via a Trp-containing linker to a C-terminal degron tag, leading to protein degradation and a low fluorescence signal. The Trp codon (TGG) encodes the cytosine base editor target sequence CCA on the transcriptional template strand. Deamination of cytosine to thymine by rAPOBEC1 converts the Trp codon into a STOP codon (UAG, UGA, or UAA, transcribed as CUA, UCA, or UUA on the template strand). This prevents degron translation, restores sfGFP expression, and results in a high fluorescence signal.

FIG. 12I are bar and box plots showing Sequence Display results and validation for UGI. The average mutation number in barcodes was used to evaluate the activity of variants in the 2NNK UGI library. Leu58 and Tyr65 were selected for the 2NNK UGI library. Five variants and WT proteins of UGI were selected for NGS validation (n=4) and degron fluorescence validation (n=6).

FIG. 12J is a graphical depiction of the plasmid design of the Sequence Display platform for UGI evolution via random mutagenesis. The recording plasmid encodes the UGI error-prone library, the corresponding base editor components, and recording barcodes with PAM sequences. The target plasmid encodes the sgRNA and complementary DNA harboring the recording barcode, directing the base editor to introduce mutations at the barcode locus.

FIG. 12K is a bar graph showing Sequence Display results for the UGI error-prone library. The average mutation number within recording barcodes was used as a quantitative proxy for UGI activity.

FIG. 12L is a box plot and bar graph depicting mutation numbers and fluorescence values of UGI error-prone variants. Nine randomly selected UGI variants and UGI-WT were subjected to NGS-based validation (n=4) and degron-based fluorescence assays (n=4, WT n=9).

FIG. 13A is a graphical representation of the function and mechanism of rAPOBEC1.

FIG. 13B is a graphical depiction of the docking structure of rAPOBEC1 with ssDNA. The rAPOBEC1 structure was predicted using AlphaFold2, while the ssDNA was derived from the hAPOBEC3A structure (PDB: 5SWW) for docking. The zoom-in interface view highlights Tyr120 and His121 as key residues interacting with ssDNA.

FIG. 13C is a graphical representation of the plasmid design of the Sequence Display platform for protein evolution of rAPOBEC1.

FIG. 13D is a graphical representation of the plasmid design for degron fluorescence assay to validate the activity of rAPOBEC1 variants. One plasmid encodes sfGFP fused to a degron tag via a 20 bp target DNA sequence and expresses the sgRNA. Another plasmid encodes dSlugCas9, UGI, and rAPOBEC1 variants.

FIG. 13E is a set of bar graphs and box plots showing Sequence Display results and validation for rAPOBEC1. The average mutation number in barcodes was used to evaluate the activity of variants in the rAPOBEC1 library.Asn984 and Lys1016 for the 2NNK SlugCas9 library, and Tyr120 and His121 was selected for the 2NNK rAPOBEC1 library. Five variants and WT proteins of rAPOBEC1 were selected for NGS validation (n=4) and degron fluorescence validation (n=6).

FIG. 14A is a graphical representation of the pipeline for evolving MbPylRS(IPYE) variants toward distinct ncAAs using the Sequence Display platform. The recording plasmid encodes an MbPylRS(IPYE) variant library and a recording barcode containing five editable cytosine sites adjacent to PAM sequences. The writing plasmid encodes the rAPOBEC1-dSpCas9-UGI base-editing complex and an sgRNA complementary to the recording barcode. A TAG (amber) codon was introduced at the second position of rAPOBEC1 (immediately after the initiator methionine) to render base editing dependent on ncAA incorporation. Genetic code expansion enables site-specific incorporation of ncAAs at amber stop codons via engineered aaRS/tRNA pairs. Here, propionyllysine (PrK), butyrylysine (BuK), or acetyllysine (AcK) is incorporated into rAPOBEC1 to restore its activity, thereby enabling barcode editing. The average mutation number in barcodes provides a quantitative readout of MbPylRS(IPYE) activity toward each ncAA. NGS links MbPylRS(IPYE) variant sequences with their corresponding barcode-derived activities to generate ncAA-specific sequence-activity datasets. These datasets can be used to train ML models that capture sequence-function relationships, enabling identification of variants with broadened ncAA recognition as well as variants exhibiting ncAA-specific preferences.

FIG. 14B is a graphical depiction of the structures of PrK, BuK and AcK. PrK, BuK and AcK are non-canonical amino acids that mimic lysine post-translational modifications and represent acylated lysine derivatives.

FIG. 14C is a graphical depiction of the docking structure of MbPylRS(IPYE) with PrK. The MbPylRS(IPYE) structure was predicted using AlphaFold2. The zoom-in interface view highlights Leu270, Tyr271, Leu274 and Cys313 as key residues interacting with PrK.

FIG. 14D is a graphical depiction of a fluorescence assay for validating the activity of aaRS variants. The incorporating plasmid encodes MbPylRS(IPYE) variants and MbPylT, while the reporting plasmid carries sfGFP(Y151TAG). In the presence of 2 mM ncAA, functional variants incorporate ncAA at the amber codon (position 151) to generate full-length sfGFP. Non-functional variants fail to incorporate ncAA, resulting in truncated sfGFP with low fluorescence. Thus, fluorescence intensity reflects the incorporation efficiency of MbPylRS(IPYE) variants toward this specific ncAA.

FIG. 14E is a bar graph showing Sequence Display results for the 2NNK MbPylRS(IPYE) library. The average mutation number in barcodes was used to evaluate the activity of variants in the 2NNK MbPylRS(IPYE) library. Tyr271 and Cys313 were selected for the 2NNK MbPylRS(IPYE) library.

FIG. 14F is a bar chart showing fluorescence results for 2NNK MbPylRS(IPYE) variants. Each variant was assayed in the presence of 2 mM PrK (n=4), with no-PrK groups (n=4) serving as negative controls.

FIG. 14G is a graphical depiction of the Sequence Display-enabled machine learning pipeline for predicting MbPylRS(IPYE) activity toward different ncAAs. Three focused 3NDT libraries (Library 1: Leu270, Tyr271, Cys313; Library 2: Tyr271, Leu274, Cys313; Library 3: Leu270, Tyr271, Leu274) were designed based on docking results shown in FIG. 8B. These libraries were pooled and subjected to Sequence Display reactions in the presence of individual ncAAs. Following NGS, ncAA-specific sequence-activity datasets were generated and used to train pLMs (ESM-2 and SaProt) coupled with downstream MLPs. A 5-fold ensemble strategy was then applied. All combinations of the four targeted residues in MbPylRS(IPYE) were evaluated by activity inference, and the top-ranked variants were selected for fluorescence-based validation.

FIGS. 14H-14J are bar charts showing fluorescence validation of inferred MbPylRS(IPYE) variants toward PrK (FIG. 14H), BuK (FIG. 14I), and AcK (FIG. 14J). Inferred MbPylRS(IPYE) variants were evaluated by fluorescence assays in the presence of 2 mM ncAA (n=6). Corresponding no-ncAA conditions (n=6) were included as negative controls. Statistical significance is indicated as P<0.05 (*), P<0.01 (**), P<0.001 (***), P<0.0001 (****), and ns (not significant).

FIG. 15A is a graphical depiction of the pipeline for evolving SlugCas9 variants with broadened PAM recognition using the Sequence Display platform. rAPOBEC1 fused with a focused library of SlugCas9 variants are incorporated into a recording plasmid containing four recording barcodes, each followed by a different PAM (NNGA, NNGT, NNGC, NNGG). A targeting plasmid containing sgRNA and complementary DNA sequences directs the base editor system to introduce mutations specifically at six cytosine sites within each recording barcode. The rAPOBEC1-SlugCas9 complexes bind these recording barcodes and introduce mutations. The average mutation number in barcodes quantitatively reflects the activity of SlugCas9 variants towards each PAM. NGS captures the SlugCas9 variant sequences alongside their corresponding PAM activities, generating a comprehensive sequence-activity dataset. This dataset can be used for machine learning to model the relationship between SlugCas9 sequences and their activities in four PAMs, facilitating the identification of variants with general PAM activity as well as those exhibiting specific PAM preferences.

FIG. 15B is a graphical depiction of the docking structure of SlugCas9-WT in complex with DNA containing the recording barcode and an NNGG PAM. The SlugCas9 structure was obtained from the AlphaFold Protein Structure Database (accession: AOA133QCR3), and the DNA structure was predicted using AlphaFold3. The zoomed-in view highlights five key residues—N984, S985, M990, E1012, and K1016—in SlugCas9 that interact with the PAM region of the DNA.

FIG. 15C is a graphical depiction of the TadA8e fluorescence assay for validating the activity of SlugCas9 variants. The recording plasmid contains sgRNA and sfGFP fused with a 20 bp target sequence and PAM sequence. The editing plasmid encodes TadA8e and SlugCas9 variants. sfGFP is linked via a 20 bp target sequence containing two stop codons (TGA and TAA) to an N-terminal PAM sequence. Initially, the stop codons halt sfGFP translation in the reporting plasmid, resulting in no fluorescence signal. These stop codons (TGA and TAA) correspond to adenine base-editing targets (TCA and TTA) on the transcriptional template strand. Deamination of adenine to guanine by TadA8e converts the two-stop codons into Arg and Gln codons (CGA and CAA; transcribed as TCG and TTG on the template strand). This modification allows sfGFP translation to proceed, restoring sfGFP expression and resulting in a strong fluorescence signal.

FIG. 15D are box plots showing Sequence Display results for SlugCas9-WT and variants. Evaluation of the accuracy of the Sequence Display platform in evolving SlugCas9 variants with diverse PAM recognition activities using four samples: SlugCas9-WT, SlugCas9-N984S, SlugCas9-K1016I, and the catalytically inactive SlugCas9-dead variant (n=4).

FIG. 15E is a set of bar charts showing fluorescence validation of SlugCas9-WT and variants. The accuracy of Sequence Display results was validated using a fluorescence assay, measuring the activities of SlugCas9-WT, SlugCas9-N984S, SlugCas9-K1016I, and the SlugCas9-dead variant against four PAMs and comparing these activities to Sequence Display results.

FIGS. 15F-15I are bar charts and box plots showing Sequence Display results and validations for the 2NNK SlugCas9 variants. Glu1012 and Lys 1016 were selected for constructing the 2NNK SlugCas9 library. The average mutation number in barcodes was used to evaluate variant activities across NNGA (FIG. 14F), NNGT (FIG. 14G), NNGC (FIG. 14H), and NNGG (FIG. 14I) PAMs. Five representative variants with SlugCas9-WT were selected for validation by NGS (NGS, n=4) and fluorescence assays (n=4) in four PAMs.

FIG. 16A is a graphical depiction of the plasmid design for SlugCas9-mediated genome editing. The writing plasmids encode SlugCas9 (liveCas9 variants) fused to N- and C-terminal nuclear localization signals (NLSs). The targeting plasmids encode sgRNAs and corresponding target sequences complementary to different endogenous genomic loci.

FIG. 16B is a graphical depiction of the experimental workflow for SlugCas9-mediated genome editing in HEK293T cells. Writing and targeting plasmids were co-transfected into HEK293T cells. After 3 days, genomic DNA was extracted and used to prepare amplicon libraries for NGS. Editing efficiencies were quantified by analyzing sequencing results.

FIG. 16C-16F are bar graphs showing examples of indel frequencies at endogenous target sites bearing NNGA (FIG. 16C), NNGT (FIG. 16D), NNGC (FIG. 16E), and NNGG (FIG. 16F) PAMs. For each PAM motif, three distinct genomic loci were tested (n=4). Genomic DNA was extracted three days after plasmid transfection and subjected to targeted deep sequencing. The PAM sequence for each target site is indicated above the corresponding panel.

FIG. 16G is a set of dot plots showing indel frequencies at 12 endogenous target sites in HEK293T cells. The five best-performing SlugCas9 variants identified from Sequence Display, together with the best sequencing variant, are compared with SlugCas9-WT across four PAM motifs, with three endogenous loci tested per PAM (n=4). Statistical significance is indicated as P<0.05 (*), P<0.01 (**), P<0.001 (***), P<0.0001 (****), and ns (not significant).

FIG. 17A is a graphical depiction of the plasmid design for SlugCBE-mediated base editing. The writing plasmids encode SlugCas9 nickase variants fused to Anc689 APOBEC1 at the N terminus, two UGI domains at the C terminus, and N- and C-terminal NLSs. The targeting plasmids encode sgRNAs together with their corresponding target sequences complementary to different endogenous genomic loci.

FIGS. 17B-17D are sets of bar graphs showing examples of C-to-T conversion at endogenous target sites in HEK293T cells with NNGA (FIG. 17B), NNGT (FIG. 17C), and NNGC (FIG. 17D) PAMs. For each PAM motif, three distinct genomic loci were tested (n=4).

Genomic DNA was extracted three days after plasmid transfection and subjected to targeted deep sequencing. The PAM and corresponding protospacer sequence for each target site are indicated above each panel. Statistical significance is indicated as P<0.05 (*), P<0.01 (**), P<0.001 (***), P<0.0001 (****), and ns (not significant).

FIG. 17E is a set of bar graphs showing base editing with SlugCBE variants in human cells at NNGG PAM sites. Examples of C-to-T conversion at endogenous target sites in HEK293T cells with NNGG PAMs. Three distinct genomic loci were tested for each condition (n=4). Genomic DNA was extracted three days after plasmid transfection and subjected to targeted deep sequencing. The PAM and corresponding protospacer sequence for each target site are indicated above each panel.

Appendix A and Appendix B are documents associated with the concepts outlined herein and are hereby incorporated by reference in their entirety.

Corresponding reference characters indicate corresponding elements among the view of the drawings. The headings used in the figures do not limit the scope of the claims.

### DETAILED DESCRIPTION

The following detailed description references the accompanying drawings that illustrate various aspects of the present disclosure. The drawings and description are intended to describe aspects and aspects of the present disclosure in sufficient detail to enable those skilled in the art to practice the present disclosure. Other components can be utilized and changes can be made without departing from the scope of the present disclosure. The following description is, therefore, not to be taken in a limiting sense.

### I. Overview

The present application introduces Sequence Display-Enabled Machine Learning (SDEML), an innovative platform designed to generate large-scale sequence-activity datasets for proteins. By integrating transfer learning from protein language models (pLMs) with our big amount of sequence-activity data and ensemble strategies, SDEML enables the fine-grained construction of variant-specific activity landscapes, facilitating the identification of high-activity variants. The systems outlined herein are applied to evolve Staphylococcus lugdunensis Cas9 (SlugCas9) for expanded protospacer adjacent motif (PAM) recognition, successfully identifying superior variants with enhanced activity across multiple PAMs. These findings establish SDEML as a powerful tool for constructing detailed sequence-activity landscapes and accelerating the discovery of optimized protein variants for applications in biology and medicine.

Over millions of years of evolution history, proteins have been central to the development of life, evolving diverse and specialized functions through natural selection to meet various biological demands. Nowadays, numerous artificially evolved proteins have been developed through different engineering and evolution methods in experimental approaches to meet the growing demand for proteins with tailored functions in different fields. Directed Evolution (DE), a groundbreaking technique, has transformed protein engineering by emulating the process of natural selection in the laboratory. Despite its success, DE remains labor-intensive and time-consuming, largely due to the extensive manual screening required to identify desired protein variants. High-throughput methods, such as phage display and positive and negative selection, are commonly employed to streamline protein evolution. However, these methods primarily focus on identifying the optimal protein variant, often discarding valuable information about less favorable and inactive variants that could provide critical insights for rational protein design. Understanding the relationship between protein amino acid sequences and their corresponding activities is fundamental for uncovering principles that can guide protein evolution and enable the rational design of enhanced protein properties. However, robust experimental methods for efficiently generating large-scale datasets that connect protein sequences to their activities remain unavailable, presenting a significant barrier to the systematic guidance of protein evolution.

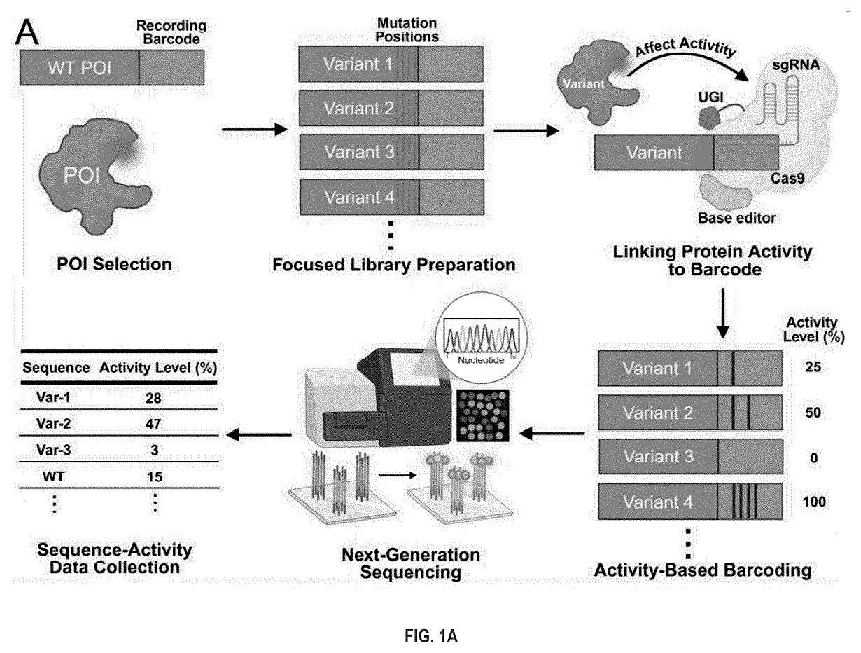

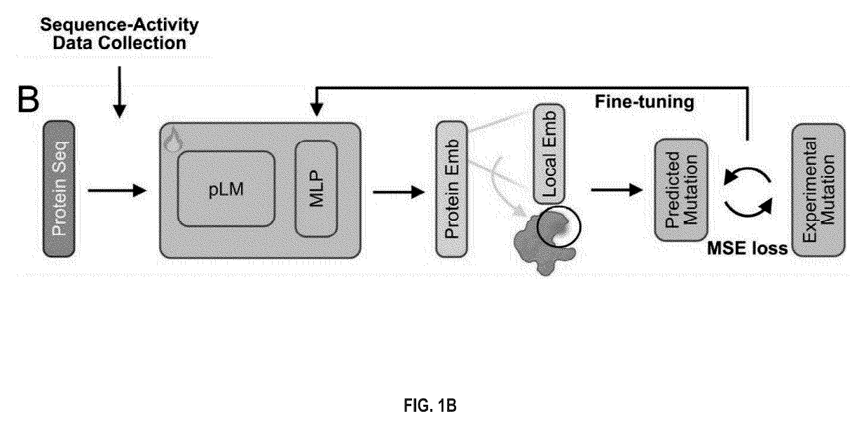

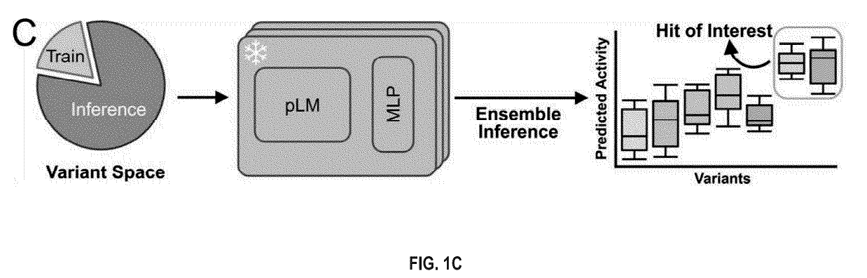

Here, the present disclosure outlines an experimental method termed Sequence Display (SD), which enables generation of large-scale protein sequence-activity datasets at the first time. This approach provides a powerful platform for systematically guiding protein evolution through data-driven insights. To implement this innovative method, a 20 bp recording barcode may be introduced adjacent to the sequence encoding the protein of interest (POI). A focused library can then be constructed based on this POI. Base editors and CRISPR/Cas9 systems were employed to specifically target this recording barcode. The activity of the POI influences the frequency of mutations introduced by the base editor within the recording barcode, allowing base changes in the barcode to serve as a quantitative measure of the activity levels of different protein variants. Following the introduction of mutations, next-generation sequencing (NGS) is used to sequence the library, capturing both the POI variants and their corresponding barcodes. By correlating the mutation numbers in the barcodes with the activities of the variants, an extensive sequence-activity dataset is generated, providing a valuable resource for guiding downstream protein evolution (FIG. 1A).

The development of protein language models (pLMs) has revolutionized the ability to capture protein information across evolutionary scales, enabling diverse downstream applications through their universal representations, which parameterize a comprehensive “protein universe”. To accelerate protein evolution, computational methods have been proposed to establish sequence-to-activity connections, leveraging pLM fitness landscapes and universal representations to evolve wild-type (WT) protein sequences. Other studies employed pLMs for novel protein design and evolution. However, despite these advancements, mapping fitness landscapes to activity landscapes using zero-shot or few-shot approaches remains limited in precision, especially when data is scarce, rendering the lack of granularity to identify optimal protein variants with the highest activity compared to experimental methods. Moreover, de novo-designed proteins frequently fail to achieve activity levels comparable to natural WT sequences. The systems outlined herein address these limitations by integrating transfer learning from pLMs with SD data, enabling a fine-grained construction of variant-specific protein activity landscapes to identify high-activity variants specific to the targeted WT (FIGS. 1B and 1C). SD experiments provide activity data for approximately 1-5% of the whole landscape, while pLMs extrapolate the remaining 95-99% through fine-tuning on this experimental data. This integration of experimental and computational approaches facilitates the creation of precise activity landscapes, offering a robust platform for efficient and flexible protein evolution.

The present disclosure further demonstrates the effectiveness and generalizability of the SDEML platform across distinct proteins, such as cytosine deaminase, uracil glycosylase inhibitor (UGI), compact Cas9 nuclease, Ribonuclease III (RNase III) and so on. These proteins play critical roles in the fields of gene editing and therapy. Furthermore, SDEML was used to evolve Staphylococcus lugdunensis Cas9 (SlugCas9) to recognize a broader NNG protospacer adjacent motif (PAM) sequence, expanding beyond the NNGG PAM recognized by the wild-type SlugCas9 (SlugCas9-WT). Also, the SDEML platform can be further used to evolve different kinds of base editors, Cas proteins, guided RNA/DNA, RNA/DNA apatamer, transcription factors, antibodies, RNA polymerases, protein inhibitors, anti-CRISPR inhibitor, inhibitors that block Protein-Protein interactions, inhibitors that block Small molecule-Protein interactions, and some other therapeutic proteins. Compared to the recently popular active learning-directed evolution (ALDE) methods, the SDEML platform outlined herein streamlines the workflow with no need for iterative optimization. The entire process, including library construction, barcode mutagenesis, NGS, and model training, can be completed within just 1-2 weeks, providing a highly efficient and time-saving approach to protein evolution. SDEML represents a revolutionary approach to protein engineering, poised to lead the development of using μl to guide protein evolution. For computational biologists, this initiative will provide more comprehensive datasets to train foundational models, which can then be used for downstream biological tasks such as structure prediction and activity prediction. For those in the field of protein engineering, it offers a new, rapid, and labor-efficient method to evolve more interesting proteins. Additionally, for those in the field of protein therapy, it will yield a greater number of promising protein candidates for pharmaceutical studies and clinical trials. Furthermore, for researchers in drug discovery, this project can accelerate the identification of novel therapeutic targets by providing a deeper understanding of protein functions and interactions. In the future, as the number of proteins evolved using the SDEML platform continues to grow, the resulting large-scale sequence-activity datasets across diverse proteins can be leveraged to develop a generalized model for protein evolution. Such a model has the potential to guide the evolution of new proteins with reduced experimental effort, streamlining the process of protein engineering.

The present application thus provides novel systems, methods, and compositions for generating large-scale, high-quality sequence-activity datasets that directly associate protein sequence information with functional activity measurements. The application is based, at least in part, on the surprising finding that protein activity can be reliably and quantitatively recorded by linking activity of a protein of interest to sequence modifications in a recording barcode, thereby enabling precise, heritable encoding of functional information without requiring survival-based selection or exhaustive screening. It was further surprisingly found that, by employing multiple recording barcodes within a single experimental framework, both activity magnitude and specificity bias of individual protein variants can be simultaneously captured, allowing reconstruction of fine-grained, variant-specific activity landscapes from only a fraction of the accessible sequence space. As described herein, this decoupled activity-to-recording architecture enables generation of extensive sequence-activity datasets even for proteins that do not inherently modify nucleic acids, and further supports integration with computational models to infer activities of unmeasured variants. Thus, the sequence display-enabled recording and analysis framework described herein is useful for accelerating protein engineering, identifying optimized protein variants, training predictive models of sequence-function relationships, and expanding the range of proteins and functional outputs amenable to systematic evolution and analysis in both biotechnological and therapeutic contexts.

### II. Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used herein, the singular forms “a,” “an,” and “the,” refer to both the singular as well as plural, unless the context clearly indicates otherwise. The abbreviation, “e.g.” is derived from the Latin exempli gratia, and is used herein to indicate a non-limiting example. Thus, the abbreviation “e.g.” is synonymous with the term “for example.”

As used herein, “comprises,” “comprising,” “containing,” and “having” and the like may have the meaning ascribed to them in U.S. Patent Law and may mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. patent law. “Consisting essentially of” or “consists essentially of” have the meaning generally ascribed to them by U.S. patent law. In particular, such terms are generally closed terms, with the exception of allowing inclusion of additional items, materials, components, steps, or elements, which do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. In this specification when using an open-ended term, like “comprising” or “including,” it is understood that direct support should be afforded also to “consisting essentially of” language as well as “consisting of” language as if stated explicitly and vice versa.

Ranges can be expressed herein as from “about” one particular value, and/or to “about” another particular value. When such a range is expressed, the range includes the bracketing values of the range as well as all values between them. It will be further understood that the endpoints of each of the ranges are significant both in relation to the other endpoint, and independently of the other endpoint. Similarly, when values are expressed as approximations, by use of the antecedent “about,” it will be understood that the disclosure encompasses the particular value. In certain example aspects, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term “about”. Further, terms used herein such as “example,” “exemplary,” or “exemplified,” are not meant to show preference, but rather to explain that the aspect discussed thereafter is merely one example of the aspect presented.

It is further to be understood that all base sizes or amino acid sizes, and all molecular weight or molecular mass values given for nucleic acids or polypeptides are approximate, and are provided for description. Although methods and materials similar or equivalent to those described herein can be used in the practice or testing of this disclosure, suitable methods and materials are described below. In case of conflict, the present specification including explanations of terms will control. In addition, the materials, methods, and examples are illustrative only and not intended to be limiting.

To facilitate review of the various aspects of this disclosure, the following explanations of specific terms are provided:

The term Nucleotide as used herein includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

The term Encoding as used herein refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those that are recombinantly or synthetically produced. In some examples, a peptide is one or more of the peptides disclosed herein.

Sequence identity: The similarity between two nucleic acid sequences, or between two amino acid sequences, is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith & Waterman Adv. Appl. Math. 2: 482, 1981; Needleman & Wunsch J. Mol. Biol. 48: 443, 1970; Pearson & Lipman Proc. Natl. Acad. Sci. USA 85: 2444, 1988; Higgins & Sharp Gene 73: 237-244, 1988; Higgins & Sharp CABIOS 5: 151-153, 1989; Corpet et al. Nuc. Acids Res. 16, 10881-90, 1988; Huang et al. Computer Appls. In the Biosciences 8, 155-65, 1992; and Pearson et al. Meth. Mol. Bio. 24, 307-31, 1994. Altschul et al. Mol. Biol. 215:403-410, 1990), presents a detailed consideration of sequence alignment methods and homology calculations.

The NCBI Basic Local Alignment Search Tool (BLAST) (Altschul et al. J. Mol. Biol. 215:403-410, 1990) is available from several sources, including the National Center for Biotechnology Information (NCBI, Bethesda, MD) and on the Internet, for use in connection with the sequence analysis programs blastp, blastn, blastx, tblastn and tblastx.

Those of skill in the art will recognize that several aspects are possible within the scope and spirit of the present disclosure. The following description illustrates the disclosure and, of course, should not be construed in any way as limiting the scope of the disclosure described herein.

### III. Systems

Disclosed herein are systems and methods for recording biological activity of protein variants in nucleic acid memory. In certain aspects, the disclosed architecture converts biological activity of a protein of interest (POI) into heritable or sequence-detectable modifications in a recording nucleic acid sequence. The modified nucleic acid sequence functions as a molecular memory encoding activity information, thereby enabling high-throughput generation of sequence-activity datasets linking genotype to phenotype.

In some aspects, the system comprises: (i) a nucleic acid encoding a variant of a POI; (ii) a recording barcode positioned in cis relative to the nucleic acid encoding the POI variant; (iii) a programmable writer enzyme capable of introducing sequence modifications into the recording barcode; and (iv) sequencing and computational analysis components for correlating barcode modifications with POI sequence.

### Recording Barcode and Cis Linkage

In certain aspects, each POI variant is associated with a recording barcode comprising a nucleic acid sequence that contains one or more editable nucleotides. The recording barcode may comprise, for example, about 5 to about 200 nucleotides, including in some aspects about 10 to about 100 nucleotides, or about 15 to about 50 nucleotides. In some aspects, the recording barcode is about 10, about 15, about 20, about 25, about 30, about 35, about 40, about 45, or about 50 nucleotides.

In some aspects, the recording barcode is positioned in cis relative to the nucleic acid sequence encoding the POI variant. As used herein, “in cis” refers to placement on the same nucleic acid molecule. In some aspects, the recording barcode and the nucleic acid encoding the POI variant are present on the same plasmid. In other aspects, they are located on the same linear DNA molecule, viral vector, integrative construct, or other recombinant nucleic acid.

In certain aspects, the recording barcode is positioned adjacent to, upstream of, downstream of, or within a defined distance (e.g., within about 10 kb, about 5 kb, about 1 kb, or about 500 bp) of the POI coding sequence. The cis configuration preserves physical linkage between genotype (POI variant sequence) and activity record (barcode modification), thereby reducing cross-association between unrelated variants.

In some aspects, each POI variant is associated with a unique recording barcode. In other aspects, a common recording barcode sequence is used across multiple variants, and activity is encoded by quantitative differences in editing patterns.

The recording barcode may comprise a plurality of editable nucleotides susceptible to enzymatic modification. In some aspects, the editable nucleotides comprise cytosine residues susceptible to C-to-T substitution. In other aspects, the editable nucleotides comprise adenine residues susceptible to A-to-G substitution. In still other aspects, other programmable nucleotide modifications may be used.

### Programmable Writer Enzyme

The system further comprises a programmable writer enzyme capable of introducing sequence modifications into the recording barcode. As used herein, a “programmable writer enzyme” refers to an enzyme or enzyme complex that modifies a nucleic acid sequence at a defined or targetable location.

In certain aspects, the programmable writer enzyme comprises a base editor. In some aspects, the base editor comprises a nucleic acid deaminase fused to a programmable DNA-binding domain. In certain aspects, the programmable DNA-binding domain comprises a CRISPR-associated protein, including but not limited to a Cas nuclease or Cas nickase, which may be guided to the recording barcode by a guide RNA. In other aspects, the programmable DNA-binding domain may comprise a zinc-finger protein, TALE domain, or other sequence-recognizing protein.

In some aspects, the writer enzyme introduces substitutions without generating double-stranded breaks. In other aspects, the writer enzyme may comprise a recombinase, transposase, nickase, ligase, polymerase, or other nucleic acid-modifying enzyme capable of generating detectable sequence modifications.

In certain aspects, the writer enzyme is encoded on the same nucleic acid construct as the POI variant. In other aspects, the writer enzyme is encoded on a separate plasmid or provided in trans within a host cell.

### Functional Coupling of Biological Activity to Writer Activity

In certain aspects, biological activity of each POI variant is functionally coupled to expression, assembly, recruitment, or catalytic activation of the programmable writer enzyme.

As used herein, “functionally coupled” refers to a causal relationship in which the biological activity of a POI variant modulates the activity of the writer enzyme.

In some aspects, the biological activity regulates transcription of the writer enzyme.

For example, a transcription factor variant may activate or repress a promoter controlling writer expression.

In other aspects, the biological activity regulates post-translational assembly or activation of the writer enzyme. For example, a protease variant may cleave a regulatory linker to activate the writer enzyme. In other aspects, a protein-protein interaction may reconstitute a split writer enzyme. In other aspects, ligand binding to a receptor or antibody variant may trigger writer activation. In yet other aspects, metabolite production by an enzyme variant may activate a metabolite-responsive promoter controlling writer expression. In other aspects, incorporation of a non-canonical amino acid may restore catalytic function of a writer enzyme containing a conditional mutation.

In some aspects, the magnitude of writer activity is proportional to the biological activity of the POI variant. In other embodiments, the coupling may produce threshold-dependent or graded responses.

### Sequence Modifications as Quantitative Molecular Memory

In certain aspects, sequence modifications introduced into the recording barcode serve as quantitative molecular memory encoding activity of the POI variant. In some aspects, the memory may be represented by edit frequency (e.g., a percentage of modified nucleotides), the total number of edited nucleotides, an editing pattern, a distribution of edits across multiple barcode positions, or combinations thereof.

In certain aspects, stronger biological activity results in increased editing frequency or increased total number of edits within the recording barcode. In other aspects, distinct activity types may generate distinct edit signatures.

Because the recording barcode is physically linked in cis to the POI coding sequence, sequence modifications are retained in association with the corresponding variant. The resulting modified barcode sequence thus encodes activity information in a stable, sequence-detectable format.

### Sequencing and Computational Correlation

Following recording, nucleic acid constructs comprising the POI variant sequence and the corresponding recording barcode are subjected to sequencing.

In some aspects, sequencing comprises next-generation sequencing (NGS), long-read sequencing, nanopore sequencing, or other high-throughput sequencing techniques. In certain aspects, the POI coding sequence and the recording barcode are sequenced within a single read. In other aspects, paired-end sequencing or computational reconstruction is used.

In some aspects, sequence data are processed to determine the nucleotide sequence encoding each POI variant, the sequence of the corresponding recording barcode, and/or the presence and extent of sequence modifications.

In certain aspects, one or more processors execute instructions to correlate POI variant sequence with barcode modification metrics to generate a sequence-activity dataset. The dataset may comprise variant identifiers and associated quantitative activity values derived from barcode edit metrics.

In some aspects, the dataset is used to train machine learning models, including protein language models, to predict activity landscapes across sequence space.

### Proteins of Interest

The disclosed architecture may be implemented in prokaryotic cells, eukaryotic cells, cell-free systems, or synthetic biological platforms.

In some aspects, the POI comprises an enzyme. As used herein, an “enzyme” refers to any protein or protein complex capable of catalyzing a chemical transformation, including but not limited to covalent modification of nucleic acids, proteins, small molecules, or metabolites. In certain aspects, the enzyme exhibits catalytic activity measurable in vitro or in vivo, and such catalytic activity may be functionally coupled to activity of the programmable writer enzyme as described herein.

In some aspects, the POI comprises a protease. As used herein, a “protease” refers to any enzyme capable of cleaving a peptide bond in a substrate polypeptide. The protease may be a serine protease, cysteine protease, aspartyl protease, metalloprotease, threonine protease, or any engineered or synthetic protease variant. The protease may act on a defined peptide sequence, a naturally occurring protein substrate, or a synthetic linker sequence engineered into a regulatory construct.

In certain aspects, variants of a protease are encoded in a plasmid library, each variant being positioned in cis with a recording barcode as described herein. Biological activity of each protease variant is functionally coupled to activity of a programmable writer enzyme such that cleavage efficiency or substrate specificity modulates the extent of sequence modification within the corresponding recording barcode.

In some aspects, protease activity regulates writer enzyme activation through cleavage of a regulatory fusion protein. For example, a transcriptional activator or RNA polymerase may be maintained in an inactive state by fusion to an inhibitory domain via a peptide linker comprising a candidate protease cleavage sequence. In certain aspects, the linker comprises a substrate sequence recognized by the protease variant library. Upon proteolytic cleavage of the linker by an active protease variant, the inhibitory domain is separated from the activator, thereby restoring transcriptional activity and inducing expression of the programmable writer enzyme. The resulting writer activity introduces sequence modifications into the cis-linked recording barcode in an activity-dependent manner.

In other aspects, protease-mediated cleavage modulates a split writer enzyme architecture. For example, two fragments of a programmable writer enzyme may be connected by a protease-sensitive linker that prevents proper folding or assembly. Cleavage of the linker by an active protease variant may permit reconstitution of the writer enzyme, leading to barcode editing. Variants exhibiting higher catalytic efficiency may produce increased barcode modification relative to variants with reduced activity.

In some aspects, protease activity modulates stability of a transcriptional repressor or activator. For example, cleavage of a degradation tag, repressor domain, or inhibitory peptide by an active protease variant may alter expression levels of the writer enzyme. In such embodiments, the magnitude of barcode modification reflects the catalytic efficiency, substrate recognition specificity, or kinetic parameters (e.g., k_{cat} or k_{cat}/K_{M}) of the protease variant.

In certain aspects, the protease is a viral protease, bacterial protease, mammalian protease, or engineered synthetic protease. The protease may comprise, for example, a serine protease such as trypsin or a chymotrypsin-like enzyme, a cysteine protease such as TEV protease, a caspase, or a viral main protease. In some aspects, the protease may be an engineered variant evolved for altered substrate specificity.

In some aspects, a protease variant library may be configured such that cleavage of a peptide linker positioned between a T7 lysozyme and a T7 RNA polymerase restores T7 RNA polymerase activity. In such aspects, active protease variants cleave the linker sequence, releasing T7 RNA polymerase from lysozyme-mediated inhibition. The restored polymerase drives transcription of a base-editing complex from a T7 promoter, thereby inducing activity-dependent editing of the recording barcode. Variants with reduced cleavage activity maintain inhibition of T7 RNA polymerase and result in correspondingly lower barcode modification levels.

In some aspects, the POI comprises a polymerase. As used herein, a “polymerase” refers to an enzyme capable of catalyzing template-directed synthesis of nucleic acid polymers, including DNA polymerases, RNA polymerases, reverse transcriptases, and RNA-dependent RNA polymerases. The polymerase may be naturally occurring, engineered, or synthetically derived, and may be modified to alter promoter recognition, substrate specificity, fidelity, processivity, or regulatory properties.

In certain aspects, variants of a polymerase are encoded in a nucleic acid construct positioned in cis with a recording barcode as described herein. Biological activity of each polymerase variant is functionally coupled to activity of a programmable writer enzyme such that polymerase-dependent transcription or replication modulates the extent of sequence modification within the corresponding recording barcode.

In some aspects, the polymerase comprises a DNA-dependent RNA polymerase. For example, variants of bacteriophage RNA polymerases, including T7 RNA polymerase or related single-subunit polymerases, may be used. In such embodiments, the writer enzyme may be operably linked to a promoter selectively recognized by the polymerase of interest. Polymerase variants exhibiting increased transcriptional activity from the polymerase-dependent promoter may drive increased expression of the programmable writer enzyme, resulting in increased editing of the recording barcode. Conversely, polymerase variants with reduced activity may produce reduced writer expression and correspondingly lower barcode modification levels.

In certain aspects consistent with the architecture described herein, polymerase activity is modulated through a regulatory fusion protein. For example, a T7 RNA polymerase may be fused to an inhibitory domain such as T7 lysozyme via a peptide linker. In such embodiments, activity of a POI that influences polymerase release or activation may indirectly regulate transcription of a base-editing complex. Variants of the polymerase that differ in promoter affinity, transcriptional initiation efficiency, elongation rate, or resistance to inhibition may therefore produce quantitatively distinct levels of barcode editing.

In some aspects, the polymerase comprises a DNA polymerase. DNA polymerase variants may differ in replication fidelity, mismatch tolerance, extension efficiency, or substrate selectivity. In certain aspects, DNA polymerase activity may regulate synthesis of a template encoding the programmable writer enzyme or may generate a DNA intermediate required for writer activation. For example, replication of a regulatory sequence or conversion of a single-stranded template to double-stranded form may be required for transcription of the writer enzyme, thereby coupling DNA polymerase activity to barcode modification.

In some aspects, the polymerase comprises a reverse transcriptase. Reverse transcriptase variants may differ in efficiency of complementary DNA (cDNA) synthesis from RNA templates. In certain aspects, synthesis of a cDNA encoding the programmable writer enzyme may be required for writer expression, thereby linking reverse transcription efficiency to barcode editing levels.

In some aspects, the polymerase comprises an RNA-dependent RNA polymerase. In such embodiments, replication of an RNA template encoding the writer enzyme or an essential regulatory component may be required for writer activation.

In some aspects, polymerase activity may be evaluated under different promoter sequences, template structures, nucleotide concentrations, cofactor availability, or inhibitory conditions. Multiple recording barcodes may be incorporated within a single construct to measure polymerase activity across distinct promoter variants or regulatory sequences, thereby enabling mapping of promoter specificity landscapes.

In certain aspects, sequence modifications introduced into the recording barcode quantitatively reflect polymerase activity. For example, increased transcriptional output may correlate with increased writer enzyme abundance, resulting in increased edit frequency, increased total number of edits, or distinct editing patterns within the recording barcode. The recording barcode thus serves as a molecular memory encoding polymerase performance metrics.

In some aspects, the POI comprises a CRISPR-associated nuclease. As used herein, a “CRISPR-associated nuclease” refers to a programmable nucleic acid-binding and/or nucleic acid-cleaving enzyme that forms part of a CRISPR (Clustered Regularly Interspaced Short Palindromic Repeats) system. Such nucleases may include Class 2 single-effector proteins, including but not limited to Cas9, Cas12 (Cpf1), Cas13, or engineered derivatives thereof. The nuclease may be wild-type, catalytically impaired, nickase-form, catalytically dead, or fused to regulatory or effector domains.

In certain aspects, variants of a CRISPR-associated nuclease are encoded in a nucleic acid construct positioned in cis with a recording barcode. Biological activity of each nuclease variant is functionally coupled to activity of a programmable writer enzyme such that target recognition, cleavage efficiency, binding affinity, or protospacer adjacent motif (PAM) compatibility modulates the extent of sequence modification within the corresponding recording barcode.

In some aspects, the POI comprises a SlugCas9 or a variant thereof. SlugCas9 is a compact Cas9 nuclease capable of programmable DNA targeting. Variants of SlugCas9 may differ in PAM recognition, cleavage efficiency, specificity, or activity across distinct target sequences. In some aspects, recognition of a target sequence or a particular PAM sequence by a SlugCas9 variant modulates activation of a writer enzyme. For example, binding or cleavage at a defined target locus may activate transcription of a base-editing complex, resulting in activity-dependent editing of the cis-linked recording barcode.

In some aspects, multiple recording barcodes are positioned downstream of distinct PAM sequences. In such embodiments, SlugCas9 variants exhibiting differential PAM compatibility (e.g., recognition of NNGA, NNGT, NNGC, or NNGG PAMs) may induce differential levels of barcode modification associated with each PAM, thereby generating a multiplexed sequence-activity profile for each variant. Barcode edit frequency, total number of edits, or editing pattern may quantitatively reflect nuclease activity toward each PAM sequence.

In certain aspects, the CRISPR-associated nuclease is configured to directly mediate modification of the recording barcode. For example, a nuclease or nickase fused to a deaminase may be guided by a guide RNA to introduce base substitutions within the recording barcode in a sequence-dependent manner. In such embodiments, guide RNAs corresponding to barcode-embedded protospacer sequences may direct editing of the barcode region, and activity of the nuclease variant may influence the efficiency of barcode editing.

In other aspects, the CRISPR-associated nuclease may regulate expression of the writer enzyme indirectly. For example, a nuclease variant may cleave a repressor element, disrupt a transcriptional terminator, or activate a transcriptional regulatory cascade controlling writer expression. In such embodiments, increased cleavage efficiency or improved target recognition may lead to increased writer activity and correspondingly increased barcode modification.

In some aspects, the CRISPR-associated nuclease comprises a catalytically inactive or nickase variant fused to transcriptional activation or repression domains. In such embodiments, DNA binding without cleavage may regulate expression of the writer enzyme. Variants exhibiting altered binding affinity or specificity may therefore generate distinct barcode modification profiles.

In certain aspects, the CRISPR-associated nuclease may comprise Cas9, Cas12, Cas13, Cas14, or engineered derivatives thereof. The nuclease may be configured for DNA cleavage, RNA cleavage, transcriptional regulation, base editing, prime editing, or other programmable nucleic acid modification activities.

In some aspects, large-scale variant libraries of SlugCas9 are generated and evaluated across multiple PAM contexts. In such embodiments, activity-dependent barcode modification may provide a quantitative readout of PAM recognition and editing efficiency, enabling construction of sequence-activity landscapes and identification of variants with broadened or altered PAM compatibility. Such embodiments demonstrate that CRISPR-associated nuclease activity, including sequence recognition and cleavage efficiency, can be recorded in nucleic acid memory through programmable writer-mediated barcode editing.

In certain aspects, the recording barcode comprises a plurality of editable nucleotides such that cumulative edits provide a graded or analog representation of nuclease activity. Increased nuclease activity toward a given target sequence may correlate with increased edit frequency, increased number of edited positions, or characteristic editing signatures within the barcode.

In some aspects, the POI comprises a single-guide RNA (sgRNA) or a variant thereof. As used herein, an “sgRNA” refers to a guide RNA capable of directing a cognate CRISPR-associated effector to a target nucleic acid sequence. sgRNA variants may differ in spacer sequence, protospacer complementarity, scaffold architecture, stem-loop configuration, stability, chemical modification, or other structural features that influence targeting efficiency or specificity. In certain embodiments, an sgRNA variant library is positioned in cis with a recording barcode and co-expressed with a CRISPR effector, including but not limited to Cas9, Cas12, Cas13, Cas14, a nickase, or a catalytically impaired variant thereof. Biological performance of each sgRNA variant, including on-target activity, off-target discrimination, or binding affinity, is functionally coupled to activity of a programmable writer enzyme such that effective sgRNA-guided targeting results in activity-dependent modification of the associated recording barcode.

In some aspects, sgRNA-guided binding or cleavage at a defined genomic or plasmid target regulates expression or activation of the writer enzyme. For example, sgRNA-dependent cleavage may disrupt a repressor element, activate a promoter, release a transcriptional activator, or otherwise modulate transcription of a base-editing complex, thereby converting sgRNA efficacy into quantitative barcode editing levels. In other embodiments, the CRISPR effector directly modifies the recording barcode in an sgRNA-dependent manner, wherein each sgRNA variant directs the effector to a corresponding protospacer embedded within or adjacent to the barcode region. sgRNA variants exhibiting higher on-target activity or improved specificity may generate increased barcode edit frequency, distinctive editing patterns, or greater numbers of edits, whereas poorly performing sgRNAs may yield reduced or absent modifications. Sequencing of sgRNA variant sequences together with their associated edited barcodes enables quantitative mapping of sgRNA performance across large variant libraries and supports optimization of guide RNA designs for diverse CRISPR-based applications.

In some aspects, the POI comprises a transcription factor. As used herein, a “transcription factor” refers to any protein capable of binding a nucleic acid regulatory sequence and modulating transcription of a target gene. The transcription factor may function as an activator, repressor, co-activator, co-repressor, or bifunctional regulator. The transcription factor may bind DNA directly or may associate with DNA indirectly through interaction with other regulatory proteins.

In certain aspects, variants of a transcription factor are encoded in a nucleic acid construct positioned in cis with a recording barcode as described herein. Biological activity of each transcription factor variant is functionally coupled to activity of a programmable writer enzyme such that transcriptional activation or repression modulates the extent of sequence modification within the corresponding recording barcode.

In some aspects, the programmable writer enzyme is placed under control of a transcription factor-responsive promoter. The promoter may comprise one or more cognate binding sites for the transcription factor positioned upstream of a minimal promoter. In such embodiments, transcription factor variants exhibiting stronger DNA-binding affinity, improved specificity, or enhanced activation potential may drive increased transcription of the writer enzyme, resulting in increased barcode editing. Conversely, variants with reduced DNA-binding affinity or impaired regulatory function may produce reduced writer expression and correspondingly lower levels of barcode modification.

In certain aspects, the transcription factor is a naturally occurring DNA-binding protein, including but not limited to bacterial transcriptional regulators, eukaryotic transcription factors, zinc-finger proteins, helix-turn-helix proteins, leucine zipper proteins, homeodomain proteins, or basic helix-loop-helix (bHLH) proteins. In other aspects, the transcription factor comprises an engineered DNA-binding protein.

In some aspects, the transcription factor may regulate a promoter controlling expression of a base-editing complex comprising a deaminase fused to a programmable DNA-binding domain. Increased transcriptional activation by a high-performance transcription factor variant may increase abundance of the base editor and thereby increase edit frequency within the cis-linked recording barcode.

In other aspects, the transcription factor may function as a repressor, and repression strength may inversely correlate with barcode modification. For example, variants exhibiting stronger repression may reduce writer enzyme expression and decrease barcode editing, while weaker repressors may allow higher writer expression and increased editing.

In certain aspects, transcription factor activity may depend on ligand binding, cofactor interaction, post-translational modification, dimerization, or interaction with chromatin. In such embodiments, the disclosed system may be used to evolve and/or identify transcription factor variants with altered ligand responsiveness, dynamic range, DNA-binding specificity, or regulatory strength. Barcode modification levels may provide a quantitative or semi-quantitative representation of transcriptional activity under defined environmental or cellular conditions.

In some aspects, multiple recording barcodes may be associated with promoters containing distinct binding site sequences. In such embodiments, transcription factor variants may be profiled simultaneously across multiple DNA-binding motifs, thereby generating a sequence-specificity landscape.

In some aspects, the POI comprises an aminoacyl-tRNA synthetase (aaRS). As used herein, an “aminoacyl-tRNA synthetase” refers to an enzyme that catalyzes attachment of an amino acid to its cognate transfer RNA (tRNA), thereby enabling incorporation of the amino acid during translation. The aaRS may be naturally occurring, engineered, orthogonal, or synthetically derived, and may be configured to recognize canonical or non-canonical amino acids (ncAAs).

In certain aspects, variants of an aaRS are encoded in a nucleic acid construct positioned in cis with a recording barcode as described herein. Biological activity of each aaRS variant is functionally coupled to activity of a programmable writer enzyme such that aminoacylation efficiency, substrate specificity, or ncAA incorporation efficiency modulates the extent of sequence modification within the corresponding recording barcode.

In some aspects, the aaRS comprises a pyrrolysyl-tRNA synthetase (PylRS) variant. In some aspects, the POI comprises MbPylRS(IPYE) or a variant thereof. In some aspects, the MbPylRS(IPYE) is engineered to recognize and incorporate non-canonical amino acids, including acylated lysine derivatives. Variants of MbPylRS(IPYE) may differ in catalytic efficiency, substrate specificity, or discrimination among ncAAs such as propionyllysine (PrK), butyrylysine (BuK), or acetyllysine (AcK).

In some aspects, activity of an aaRS variant is coupled to restoration or activation of a programmable writer enzyme via ncAA-dependent translation. For example, a premature stop codon (e.g., a TAG amber codon) may be introduced at a permissive position within a base editor or within an essential component of the writer enzyme. In such embodiments, efficient charging of an orthogonal tRNA with an ncAA by a high-performance aaRS variant enables readthrough of the stop codon and production of a functional writer enzyme. The functional writer enzyme then introduces sequence modifications into the cis-linked recording barcode in an activity-dependent manner.

In some aspects, MbPylRS(IPYE) variants are evaluated for incorporation of distinct ncAAs (e.g., PrK, BuK, or AcK). Efficient incorporation of a selected ncAA into a base editor containing an amber codon restores base-editing activity. Variants exhibiting higher incorporation efficiency generate increased barcode edit frequency, increased total number of edits, or distinct edit patterns within the recording barcode.

In some aspects, the aaRS may be evaluated under varying ncAA concentrations, in the presence of competing canonical amino acids, or across multiple ncAA substrates. Multiple recording barcodes may be incorporated within a single construct to simultaneously evaluate aaRS performance across different ncAA conditions, thereby generating ncAA-specific sequence-activity datasets.

In certain aspects, the aaRS may be part of an orthogonal translation system (OTS) comprising an engineered aaRS and a cognate tRNA that do not cross-react with endogenous host translational machinery. The OTS may be introduced into a prokaryotic or eukaryotic host cell. In such embodiments, aaRS activity may be measured in bacterial cells, mammalian cells, or cell-free expression systems.

In some aspects, aaRS variants may be generated by site-saturation mutagenesis, focused mutagenesis of active-site residues, combinatorial mutagenesis, or error-prone PCR. For example, residues within the amino acid binding pocket of MbPylRS(IPYE), such as Leu270, Tyr271, Leu274, or Cys313, may be randomized to generate variant libraries. Barcode editing levels may provide a quantitative measure of substrate recognition and aminoacylation efficiency for each variant.

In certain aspects, sequence modifications introduced into the recording barcode may serve as quantitative molecular memory encoding aaRS activity. For example, increased aminoacylation efficiency or improved ncAA specificity may correlate with increased edit frequency or total number of edits within the barcode. Conversely, variants with reduced charging efficiency may generate fewer edits.

In some aspects, the POI comprises a binding protein or antibody. As used herein, a “binding protein” refers to any protein capable of specifically associating with a target molecule, including proteins that recognize peptides, proteins, nucleic acids, carbohydrates, lipids, small molecules, metabolites, post-translational modifications, or other ligands. As used herein, an “antibody” includes full-length immunoglobulins, antibody fragments, engineered antibody formats, and antibody-derived scaffolds.

In certain aspects, the POI comprises an antibody or antibody fragment, including but not limited to IgG, Fab, scFv, single-domain antibodies (e.g., VHH or nanobodies), bispecific antibodies, or engineered affinity reagents. Variants of such antibodies may differ in binding affinity, specificity, epitope recognition, stability, or functional activity.

In some aspects, ligand binding by a POI binding protein or antibody is functionally coupled to activation or modulation of a programmable writer enzyme. For example, binding of a target antigen may induce proximity-based reconstitution of a split writer enzyme, activate a ligand-responsive transcription factor controlling writer expression, or trigger recruitment of a writer enzyme to a defined recording locus. In such embodiments, stronger binding affinity or improved specificity may result in increased barcode editing, whereas weaker or non-binding variants may generate reduced or absent barcode modification.

In certain aspects, binding may regulate transcription of the writer enzyme through receptor signaling pathways, dimerization-dependent transcriptional activation, or displacement of inhibitory complexes. In other aspects, binding of a target molecule may stabilize or destabilize a regulatory fusion protein controlling writer activity.

In some aspects, the disclosed system may be used to generate sequence-activity datasets for antibody libraries to identify improved binders, altered specificity variants, or variants with enhanced functional properties such as receptor blockade or activation.

In some aspects, the POI comprises a metabolic enzyme or a variant thereof. As used herein, a “metabolic enzyme” refers to any enzyme that catalyzes a biochemical transformation within a metabolic pathway, including enzymes involved in central carbon metabolism, amino acid biosynthesis, nucleotide metabolism, lipid metabolism, redox metabolism, secondary metabolite biosynthesis, or engineered synthetic pathways. Variants of a metabolic enzyme may differ in catalytic efficiency, substrate specificity, cofactor utilization, regulatory properties, stability, expression level, or pathway flux contribution. In certain aspects, a variant library of a metabolic enzyme is positioned in cis with a recording barcode and expressed in a host cell such that metabolic output or pathway activity can be functionally coupled to activity of a programmable writer enzyme.

In some aspects, biological activity of the metabolic enzyme is coupled to barcode modification through a metabolite-responsive sensing module. For example, formation of a product metabolite, accumulation of an intermediate, or depletion of a substrate may activate a transcription factor, riboswitch, two-component sensor, ligand-inducible repressor or activator, or other regulatory element controlling expression or activation of a programmable writer enzyme. In such embodiments, enzyme variants that produce higher levels of a target metabolite or increase pathway throughput may induce stronger writer activity and correspondingly increased modification of the associated recording barcode. Conversely, variants exhibiting reduced catalytic activity or diminished pathway flux may generate reduced barcode modification. In certain embodiments, multiple recording barcodes may be associated with distinct metabolite-responsive modules to profile enzyme activity across different pathway branches or environmental conditions.

In some aspects, the sensing module may operate at the transcriptional, translational, or post-translational level. For example, metabolite binding to a transcription factor may activate transcription of a writer enzyme; a metabolite-responsive riboswitch may modulate translation efficiency of the writer enzyme; or a metabolite-dependent conformational change may activate a split or conditionally assembled writer enzyme complex. In each case, the magnitude of barcode editing (i.e., such as edit frequency, total number of edits, or characteristic edit pattern) provides a quantitative or semi-quantitative representation of metabolic enzyme performance.

Sequencing of the nucleic acid encoding each metabolic enzyme variant together with its associated modified recording barcode enables correlation of enzyme genotype with metabolic output. In certain embodiments, barcode modification metrics are used to rank variants according to catalytic efficiency, product yield, substrate utilization, or pathway flux contribution. Such embodiments permit large-scale sequence-activity mapping and high-throughput evolution of metabolic enzymes across diverse pathways, including both naturally occurring and engineered biosynthetic routes.

In some aspects, the POI comprises a riboswitch or a variant thereof. As used herein, a “riboswitch” refers to a regulatory RNA element capable of modulating gene expression in response to binding of a target ligand. A riboswitch may comprise an aptamer domain that binds a small molecule, metabolite, ion, or other ligand, and an expression platform that alters transcription, translation, RNA stability, splicing, or ribozyme-mediated cleavage in response to ligand binding. Riboswitches may be naturally occurring or synthetically engineered, and may be configured as ON-switches, OFF-switches, or graded-response regulators.

In certain aspects, a riboswitch variant library is operably linked in cis with a recording barcode and positioned in a regulatory context controlling expression and/or activity of a programmable writer enzyme. For example, each riboswitch variant may be placed within the 5′ untranslated region (UTR) of a transcript encoding a writer enzyme or an essential component thereof. In some embodiments, ligand binding to the riboswitch modulates ribosome binding site accessibility, transcriptional termination, RNA stability, alternative splicing, or ribozyme cleavage, thereby altering expression or functional assembly of the writer enzyme. In other embodiments, the riboswitch may regulate expression of an upstream activator or repressor that indirectly controls writer activity.

In certain aspects, riboswitch variants exhibiting stronger ligand-dependent activation may produce increased writer enzyme output and correspondingly increased modification of the associated recording barcode. Conversely, variants exhibiting tighter repression, reduced leakiness, or altered ligand specificity may produce decreased barcode editing in the absence or presence of ligand, depending on circuit design. In some aspects, barcode modification metrics-such as edit frequency, total number of edits, or edit pattern-serve as a quantitative or semi-quantitative representation of riboswitch performance, including dynamic range, ligand sensitivity, and regulatory precision.

Following a recording phase, sequencing of nucleic acid constructs comprising the riboswitch variant and its associated modified recording barcode enables correlation of riboswitch sequence with functional output. In certain embodiments, barcode modification levels are used to rank riboswitch variants according to ligand responsiveness, specificity, dynamic range, or background activity. Such embodiments permit large-scale sequence-activity mapping of riboswitch libraries and facilitate optimization of ligand-responsive gene control systems for use in prokaryotic, eukaryotic, or synthetic biological contexts.

In some aspects, the POI comprises an aptamer or a variant thereof. As used herein, an “aptamer” refers to a nucleic acid molecule, including RNA or DNA, capable of specifically binding a target molecule. Aptamers may bind proteins, peptides, nucleic acids, small molecules, metabolites, ions, carbohydrates, lipids, or other ligands. Aptamers may be naturally occurring, selected in vitro (e.g., via SELEX or related techniques), or synthetically engineered. Variants of an aptamer may differ in binding affinity, specificity, structural stability, folding kinetics, or ligand responsiveness.

In certain aspects, an aptamer variant library is positioned in cis with a recording barcode and configured such that target binding by the aptamer modulates activity of a programmable writer enzyme. In some embodiments, the aptamer is incorporated within a transcript encoding the writer enzyme or a regulatory component thereof. Ligand binding may induce a conformational change that alters transcription, translation, RNA stability, ribozyme cleavage, or splice-site accessibility, thereby increasing or decreasing expression of the writer enzyme. In other aspects, target binding by the aptamer promotes proximity-based reconstitution of a split transcriptional activator, such as a split RNA polymerase, or facilitates recruitment of a barcode-writing enzyme to the recording locus through aptamer-protein interactions. In still other aspects, ligand binding stabilizes an “ON” conformation that exposes a ribosome-binding site, promoter element, or other regulatory feature controlling writer output.

In certain aspects, aptamer variants exhibiting stronger binding affinity or improved specificity for a target ligand produce increased writer enzyme activity and correspondingly increased modification of the associated recording barcode. Conversely, weakly binding or non-binding variants may yield reduced or absent barcode modification. Barcode modification metrics, including edit frequency, total number of edits, or characteristic edit patterns, may provide a quantitative or semi-quantitative representation of aptamer binding performance and switching behavior.

Following a recording phase, sequencing of nucleic acid constructs comprising the aptamer variant and its associated modified recording barcode enables correlation of aptamer sequence with functional output. In certain aspects, barcode modification levels are used to rank aptamer variants according to affinity, specificity, dynamic range, or ligand-dependent regulatory properties. Such embodiments permit large-scale sequence-activity mapping of aptamer libraries and facilitate identification and optimization of aptamers for sensing, regulatory, diagnostic, or therapeutic applications.

In some aspects, the POI comprises an anti-CRISPR (Acr) protein or a variant thereof. As used herein, an “anti-CRISPR” protein refers to a protein capable of inhibiting the activity of a CRISPR-associated effector, including nucleases, nickases, base editors, transcriptional regulators, or other programmable CRISPR-derived systems. Acr proteins may function by blocking DNA binding, inhibiting catalytic activity, preventing guide RNA loading, promoting effector degradation, or otherwise interfering with CRISPR effector function. Variants of an Acr protein may differ in inhibitory potency, specificity toward different CRISPR effectors, binding affinity, stability, or regulatory properties.

In certain aspects, an Acr variant library is positioned in cis with a recording barcode and co-expressed with a CRISPR effector configured to modulate barcode writing. In some embodiments, the circuit architecture is designed such that CRISPR effector activity suppresses activity of a programmable writer enzyme. For example, the CRISPR effector may be directed to repress transcription of a writer enzyme by targeting its promoter or coding sequence, to cleave a regulatory element required for writer expression, or to interfere with transcription or translation of the writer enzyme. In other aspects, the CRISPR effector may directly target the recording barcode to prevent editing, such as by cleavage, nicking, transcriptional interference, or blocking access of the writer enzyme to the barcode region.

In such configurations, effective Acr variants that inhibit the CRISPR effector relieve suppression of the writer enzyme and thereby restore or increase barcode editing in an activity-dependent manner. Acr variants exhibiting stronger inhibitory activity may produce increased writer output and correspondingly increased barcode modification, whereas weak or inactive variants may fail to inhibit the effector, resulting in continued suppression and low or absent barcode edits. In some embodiments, the magnitude of barcode modification (i.e., such as edit frequency, total number of edits, or characteristic edit patterns) serves as a quantitative or semi-quantitative representation of Acr potency and/or specificity toward a given CRISPR effector.

Following a recording phase, sequencing of nucleic acid constructs comprising each Acr variant and its associated modified recording barcode enables correlation of Acr genotype with inhibitory performance. In certain aspects, barcode modification metrics are used to rank Acr variants according to inhibition efficiency, effector specificity, or resistance to escape mutations. Such embodiments permit large-scale sequence-activity mapping and high-throughput evolution of anti-CRISPR proteins targeting Cas9, Cas12, Cas13, or other CRISPR-derived systems.

The disclosed systems may be provided as plasmid libraries, isolated nucleic acids, vectors, host cells, or kits comprising one or more of the above components.

### IV. Methods

Also provided herein, in some aspects, is a method comprising: (a) providing a nucleic acid encoding a variant of a protein of interest and a recording barcode positioned on the same nucleic acid molecule; (b) introducing activity-dependent sequence modifications into the recording barcode using a writer enzyme; (c) sequencing the nucleic acid encoding the variant and the recording barcode; and (d) correlating the variant sequence with barcode modifications to determine an activity value for the variant.

In other aspects, provided herein is a method of generating a sequence-activity dataset for variants of a protein of interest (POI), the method comprising: (a) constructing a plasmid library comprising a plurality of plasmids, each plasmid comprising: (i) a nucleic acid sequence encoding a variant of the POI; and (ii) a recording barcode comprising a nucleic acid sequence positioned in cis with the nucleic acid sequence encoding the variant of the POI; (b) expressing the plasmid library in a cell in the presence of a writer enzyme capable of introducing one or more sequence modifications in the recording barcode; (c) coupling biological activity of each variant of the POI to expression or activity of the writer enzyme such that sequence modifications are introduced into the corresponding recording barcode in an activity-dependent manner; (d) sequencing the nucleic acid sequence encoding the variant of the POI and the corresponding recording barcode; and (e) using at least one processor in communication with a memory to correlate the nucleic acid sequence encoding the variant of the POI with sequence modifications in the corresponding recording barcode to generate a sequence-activity dataset.

### Construction of Plasmid Library

In certain aspects, the method comprises constructing a plasmid library comprising a plurality of plasmids, each plasmid comprising: (i) a nucleic acid sequence encoding a variant of the POI; and (ii) a recording barcode positioned in cis with the nucleic acid sequence encoding the variant as described hereinabove.

The plasmid library may be generated using any suitable mutagenesis strategy. In some aspects, variants of the POI are generated by site-saturation mutagenesis at one or more selected residues. In other aspects, variants are generated by error-prone PCR to introduce random mutations. In still other aspects, combinatorial mutagenesis, synthetic gene assembly, rational design, or computationally guided mutagenesis may be used. The resulting library may comprise at least about 10², 10³, 10⁴, or more distinct POI variants. In some aspects, the plasmid library may comprise between about 10² and about 10⁶ distinct POI variants, such as between about 10² and about 10⁵, between about 10² and about 10⁴, or between about 10² and about 10³ distinct POI variants.

In some aspects, each plasmid comprises a unique recording barcode sequence associated with a single POI variant. In other aspects, a common barcode sequence is used, and activity differences are reflected in quantitative modification levels rather than barcode identity.

### Expression in Host Cells

In certain aspects, the plasmid library is introduced into a host cell for expression. The host cell may be a prokaryotic cell, such as Escherichia coli, or a eukaryotic cell, including yeast, insect, or mammalian cells. In some aspects, the host cell comprises regulatory elements suitable for controlled expression of the POI variants and the programmable writer enzyme.

In some aspects, transformation conditions are selected to favor introduction of a single plasmid variant per cell, thereby preserving one-to-one correspondence between POI variant and barcode within individual cells.

### Accumulation of Activity-Dependent Sequence Modifications

In certain aspects, the writer enzyme introduces one or more sequence modifications into the recording barcode over a defined period of expression. Activity-dependent modifications may accumulate such that the number, frequency, or pattern of edits within the barcode reflects biological activity of the corresponding POI variant.

In some aspects, stronger biological activity results in increased edit frequency or increased total number of edits within the barcode. In other embodiments, distinct activity classes may generate characteristic editing signatures.

### Sequencing of POI and Recording Barcode

Following the recording phase, nucleic acid constructs comprising the POI coding sequence and the associated recording barcode are subjected to sequencing.

In some aspects, amplification of the region containing both the POI coding sequence and the barcode is performed prior to sequencing. Sequencing may comprise next-generation sequencing, long-read sequencing, nanopore sequencing, or other suitable techniques. In certain aspects, both the POI coding sequence and the recording barcode are obtained within a single sequencing read or through paired-end reconstruction.

### Computational Correlation and Dataset Generation

In certain aspects, sequence data are processed using one or more processors configured to identify POI variant sequences and corresponding barcode modifications as described herein. Computational steps may include alignment, quality filtering, counting of edited nucleotides, and calculation of edit frequency or total edit number.

In some aspects, barcode modification metrics are used to calculate a quantitative activity value for each POI variant. The resulting data may be compiled into a sequence-activity dataset comprising a plurality of POI variant sequences and associated activity measurements.

In certain aspects, the sequence-activity dataset may be used for downstream analyses, including statistical modeling, construction of activity landscapes, or training of machine learning models configured to predict functional properties of additional variants.

Also provided herein, in some aspects, is a method of recording biological activity of protein variants in nucleic acid memory, the method comprising: (a) providing a plurality of nucleic acid constructs, each construct comprising: (i) a nucleic acid sequence encoding a variant of a protein of interest (POI); and (ii) a recording sequence comprising one or more editable nucleotides; (b) functionally coupling biological activity of each variant of the POI to activity of a programmable writer enzyme such that the writer enzyme modifies the recording sequence in proportion to or in response to the biological activity of the variant; (c) permitting the writer enzyme to introduce one or more sequence modifications into the recording sequence; (d) determining the sequence of the nucleic acid sequence encoding the variant of the POI and the sequence of the modified recording sequence; and (e) correlating, using at least one processor, the sequence of the variant of the POI with the sequence modifications in the recording sequence to generate a dataset associating protein sequence with recorded biological activity.

In some aspects, sequence modifications introduced into the recording sequence serve as quantitative molecular memory of POI activity. In some aspects, the recording may be represented by the frequency of modification at one or more positions, the total number of modified nucleotides, the pattern or distribution of modifications, the presence or absence of recombination events, and/or the structural alterations detectable by sequencing.

In some aspects, accumulation of sequence modifications over time provides an analog or graded record of biological activity. In other embodiments, discrete sequence changes may represent categorical activity states.

In some aspects, because the recording sequence is positioned in cis with the POI coding sequence, the modified recording sequence remains physically associated with the genetic identity of the POI variant. This cis linkage preserves genotype-phenotype association through sequencing.

### V. Computer Implemented Systems

FIG. 10 is a schematic block diagram of an example device 100 that may be used with one or more embodiments described herein, e.g., as a component implementing aspects of the SDEML system outlined herein and in FIGS. 1A-5.

Device 100 comprises one or more network interfaces 110 (e.g., wired, wireless, PLC, etc.), at least one processor 120, and a memory 140 interconnected by a system bus 150, as well as a power supply 160 (e.g., battery, plug-in, etc.). Device 100 can also include or otherwise communicate with a display interface device 130 which can include one or more input/output devices that enable a user to input data, and to view or otherwise access output data. Input/output devices can include but are not limited to a monitor, a touch-screen, a speaker, a keyboard, a mouse, and the like.

Network interface(s) 110 include the mechanical, electrical, and signaling circuitry for communicating data over the communication links coupled to a communication network. Network interfaces 110 are configured to transmit and/or receive data using a variety of different communication protocols. As illustrated, the box representing network interfaces 110 is shown for simplicity, and it is appreciated that such interfaces may represent different types of network connections such as wireless and wired (physical) connections. Network interfaces 110 are shown separately from power supply 160, however it is appreciated that the interfaces that support PLC protocols may communicate through power supply 160 and/or may be an integral component coupled to power supply 160.

Memory 140 includes a plurality of storage locations that are addressable by processor 120 and network interfaces 110 for storing software programs and data structures associated with the embodiments described herein. In some embodiments, device 100 may have limited memory or no memory (e.g., no memory for storage other than for programs/processes operating on the device and associated caches). Memory 140 can include instructions executable by the processor 120 that, when executed by the processor 120, cause the processor 120 to implement aspects of the systems and the methods outlined herein.

Processor 120 comprises hardware elements or logic adapted to execute the software programs (e.g., instructions) and manipulate data structures 145. An operating system 142, portions of which are typically resident in memory 140 and executed by the processor, functionally organizes device 100 by, inter alia, invoking operations in support of software processes and/or services executing on the device. These software processes and/or services may include SDEML processes/services 190, which can include aspects of the methods and/or implementations of various modules described herein. Note that while SDEML processes/services 190 is illustrated in centralized memory 140, alternative embodiments provide for the process to be operated within the network interfaces 110, such as a component of a MAC layer, and/or as part of a distributed computing network environment.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the SDEML processes/services 190 is shown as a standalone process, those skilled in the art will appreciate that this process may be executed as a routine or module within other processes.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

### VI. Libraries, Plasmids, Vectors, and Host Cells

In certain aspects, the disclosed systems and methods utilize a plasmid library comprising a plurality of recombinant nucleic acid constructs, each construct encoding a variant of a protein of interest (POI) and a recording sequence positioned in cis relative to the POI coding sequence.

As used herein, a “plasmid library” refers to a population of plasmids comprising distinct nucleic acid sequences encoding different POI variants. In some embodiments, the library comprises at least about 10² distinct variants. In other embodiments, the library comprises at least about 10³, 10⁴, 10⁵, or more distinct variants. In certain embodiments, the practical upper limit of library size is determined by transformation efficiency, cloning efficiency, or host cell capacity.

Each plasmid in the library may comprise: a nucleic acid sequence encoding a variant of the POI; a recording sequence comprising one or more nucleotides susceptible to modification by a programmable writer enzyme; and/or one or more regulatory elements controlling expression of the POI and/or writer enzyme.

The recording sequence may be positioned upstream of, downstream of, or within a defined proximity to the POI coding sequence, provided that the recording sequence and the POI variant remain physically linked on the same nucleic acid molecule.

In some aspects, each plasmid comprises a unique recording barcode corresponding to a single POI variant. In other embodiments, a common recording sequence may be used across variants, and activity information is encoded quantitatively through modification patterns.

In certain aspects, POI variant libraries are generated using site-saturation mutagenesis. Site-saturation mutagenesis may target one or more selected residues within a catalytic site, binding interface, regulatory domain, or structurally significant region.

Degenerate codons (e.g., NNK, NNS, NDT, or other degenerate codon schemes) may be used to encode a defined subset or full complement of amino acids at selected positions.

In other aspects, libraries are generated by error-prone PCR, which introduces random nucleotide substitutions at a controlled mutation rate. Error-prone amplification may be performed under conditions that increase misincorporation frequency, including altered divalent cation concentrations or use of low-fidelity polymerases.

In certain aspects, combinatorial mutagenesis may be performed by assembly of synthetic oligonucleotides encoding predefined mutation combinations. In other embodiments, fully synthetic gene libraries may be generated using parallel DNA synthesis platforms.

In some aspects, computationally designed libraries may be constructed based on structural modeling, evolutionary analysis, or machine learning-guided residue selection. The disclosed systems are not limited to any particular mutagenesis or library construction strategy.

In certain aspects, the disclosed compositions comprise isolated nucleic acids encoding a POI variant and a recording sequence positioned in cis.

As used herein, an “isolated nucleic acid” refers to a nucleic acid molecule that has been removed from its natural genomic context or artificially constructed through recombinant or synthetic means. The isolated nucleic acid may be DNA or RNA and may comprise double-stranded or single-stranded molecules.

In some aspects, the isolated nucleic acid comprises: a promoter operably linked to the POI coding sequence; a recording sequence positioned on the same molecule; and optional transcriptional terminators, selectable markers, and/or replication origins.

The isolated nucleic acid may be maintained episomally, integrated into a genome, or provided in vitro in a cell-free transcription-translation system.

In certain aspects, the nucleic acid encoding the POI variant and recording sequence is incorporated into a vector. As used herein, a “vector” refers to any nucleic acid construct capable of delivering or maintaining a nucleic acid sequence within a host cell.

In some aspects, the vector comprises a plasmid vector capable of autonomous replication in a host cell. The plasmid may include an origin of replication suitable for bacterial, yeast, or mammalian cells.

In other aspects, the vector comprises a viral vector. Viral vectors may include lentiviral vectors, adenoviral vectors, adeno-associated viral (AAV) vectors, retroviral vectors, or other engineered viral delivery systems.

In certain aspects, the vector comprises an integrative vector capable of genomic insertion. Integration may occur via homologous recombination, transposase-mediated insertion, recombinase-mediated integration, or other genomic integration mechanisms.

In some aspects, the vector comprises elements enabling inducible expression, conditional expression, or tissue-specific expression.

The disclosed systems are not limited to any particular vector backbone or delivery modality.

In certain aspects, expression of the POI variant is controlled by one or more regulatory elements. Such regulatory elements may include constitutive promoters, inducible promoters, minimal promoters, tissue-specific promoters, or synthetic promoters.

Inducible systems may include, for example: tetracycline-inducible systems, IPTG-inducible systems, arabinose-inducible systems, hormone-responsive promoters, and/or metabolite-responsive promoters.

In certain aspects, expression of the programmable writer enzyme is controlled independently of the POI. In other aspects, the POI and writer enzyme may be co-expressed from a single transcript separated by self-cleaving peptides (e.g., 2A peptides) or internal ribosome entry sites (IRES).

In some aspects, transcriptional terminators, ribosome binding sites, polyadenylation signals, enhancer elements, or insulator sequences may be included to regulate transcription and translation.

In certain aspects, the plasmid library or vector is introduced into a host cell for expression and recording.

In some aspects, the host cell is a prokaryotic cell, including but not limited to Escherichia coli, Bacillus species, or other bacterial strains. In certain aspects, highly competent bacterial strains are used to maximize library diversity.

In other aspects, the host cell is a eukaryotic cell, including yeast cells (e.g., Saccharomyces cerevisiae), insect cells, plant cells, or mammalian cells such as HEK293 cells, CHO cells, or other cultured cell lines.

In certain aspects, the host cell may comprise endogenous or introduced components required for function of the programmable writer enzyme, including guide RNAs, accessory proteins, or orthogonal translation systems.

In some aspects, the host cell is selected based on compatibility with the POI, the writer enzyme, or the intended application. The disclosed systems are not limited to any specific host organism.

### VI. Kits

In some aspects, provided herein is a kit for generating a sequence-activity dataset for variants of a protein of interest (POI), the kit comprising: a) a plasmid library comprising a plurality of plasmids, each plasmid comprising: i) a nucleic acid sequence encoding a variant of the POI; and ii) a recording barcode comprising one or more editable nucleotides positioned on the same plasmid as the nucleic acid sequence encoding the variant of the POI; b) a nucleic acid construct encoding a programmable writer enzyme capable of introducing sequence modifications into the recording barcode; and c) one or more reagents for expressing the plasmid library and the programmable writer enzyme in a host cell.

In some aspects, the kit further comprises instructions for coupling biological activity of variants of the POI to activity of the programmable writer enzyme to generate activity-dependent sequence modifications in the recording barcode.

In some aspects, the kit further comprises instructions for sequencing the nucleic acid sequence encoding the variants of the POI and the corresponding recording barcodes.

In some aspects, the kit further comprises instructions for correlating variant sequences with barcode modifications to generate a sequence-activity dataset.

VIII. Sequences

| Nickname | Residues | SEQ ID |
|---|---|---|
| rAPOBEC1- | ATGTCTTCTGAAACCGGTCCGGTTGCGGTTGACC | SEQ ID NO: 1 |
| SlugCas9- | CGACCCTGCGTCGTCGTATCGAACCGCACGAATT | |
| UGI complex | CGAAGTTTTCTTCGACCCGCGTGAACTGCGTAAA | |
| | GAAACCTGCCTGCTGTACGAAATCAACTGGGGT | |
| | GGTCGTCACTCTATCTGGCGTCACACCTCTCAGA | |
| | ACACCAACAAACACGTTGAAGTTAACTTCATCGA | |
| | AAAATTCACCACCGAACGTTACTTCTGCCCGAAC | |
| | ACCCGTTGCTCTATCACCTGGTTCCTGTCTTGGTC | |
| | TCCGTGCGGTGAATGCTCTCGTGCGATCACCGAA | |
| | TTCCTGTCTCGTTACCCGCACGTTACCCTGTTCAT | |
| | CTACATCGCGCGTCTGTACCACCACGCGGACCCG | |
| | CGTAACCGTCAGGGTCTGCGTGACCTGATCTCTT | |
| | CTGGTGTTACCATCCAGATCATGACCGAACAGGA | |
| | ATCTGGTTACTGCTGGCGTAACTTCGTTAACTAC | |
| | TCTCCGTCTAACGAAGCGCACTGGCCGCGTTACC | |
| | CGCACCTGTGGGTTCGTCTGTACGTTCTGGAACT | |
| | GTACTGCATCATCCTGGGTCTGCCGCCGTGCCTG | |
| | AACATCCTGCGTCGTAAACAGCCGCAGCTGACCT | |
| | TCTTCACCATCGCGCTGCAGTCTTGCCACTACCA | |
| | GCGTCTGCCGCCGCACATCCTGTGGGCGACCGGT | |
| | CTGAAATCCGGTAGCGAAACACCGGGGACTTCA | |
| | GAATCGGCCACCCCGGAGTCTATGAATCAAAAA | |
| | TTCATTCTGGGGTTGGCTATTGGAATCACGTCGG | |
| | TGGGGTATGGCCTTATTGACTATGAAACCAAGAA | |
| | CATCATTGACGCGGGGGTCCGTTTGTTCCCCGAG | |
| | GCCAATGTCGAAAATAACGAGGGCCGTCGTAGT | |
| | AAACGCGGCTCCCGTCGTCTTAAACGCCGCCGCA | |
| | TCCACCGTCTTGAGCGCGTGAAGAAGTTGCTGGA | |
| | GGATTATAACCTGCTTGATCAGAGCCAGATCCCG | |
| | CAGTCTACCAATCCATATGCTATTCGTGTCAAAG | |
| | GGCTTTCTGAGGCATTATCCAAGGACGAGCTGGT | |
| | GATTGCATTACTTCACATTGCTAAACGCCGCGGT | |
| | ATTCATAAAATCGATGTAATCGATAGTAACGACG | |
| | ATGTTGGTAACGAGCTTTCGACGAAGGAACAGTT | |
| | GAACAAAAACTCGAAACTTTTGAAGGATAAGTT | |
| | CGTTTGTCAGATCCAACTTGAACGTATGAATGAA | |
| | GGTCAAGTCCGTGGAGAAAAGAATCGTTTTAAA | |
| | ACTGCCGATATTATCAAGGAAATCATCCAATTGC | |
| | TTAATGTTCAAAAAAATTTCCACCAATTAGACGA | |
| | GAATTTTATCAACAAATACATCGAACTGGTGGAG | |
| | ATGCGTCGTGAATATTTTGAAGGACCTGGAAAG | |
| | GGCAGTCCTTATGGGTGGGAAGGGGACCCCAAG | |
| | GCCTGGTACGAGACCTTAATGGGGCATTGTACTT | |
| | ATTTTCCAGATGAATTACGCTCAGTAAAGTATGC | |
| | GTATAGCGCGGACTTGTTCAACGCGTTAAATGAT | |
| | TTAAACAATCTGGTTATCCAGCGCGACGGCTTAT | |
| | CAAAGTTGGAGTACCACGAGAAATATCATATTAT | |
| | TGAAAATGTATTTAAGCAGAAGAAAAAACCGAC | |
| | CTTAAAACAAATTGCTAATGAAATTAACGTAAAT | |
| | CCAGAAGATATTAAGGGTTATCGCATCACAAAG | |
| | AGTGGCAAACCTCAGTTCACGGAGTTCAAATTAT | |
| | ACCATGATTTAAAGTCCGTTCTTTTTGACCAATC | |
| | TATTTTGGAAAACGAAGATGTGCTTGATCAGATT | |
| | GCTGAAATCTTAACGATTTACCAAGATAAAGATT | |
| | CAATTAAATCGAAGTTAACTGAGTTGGATATTTT | |
| | ACTGAATGAAGAAGATAAGGAGAACATCGCTCA | |
| | ACTGACCGGATACACCGGTACTCATCGCTTATCC | |
| | TTAAAGTGCATTCGCTTGGTGTTGGAAGAACAAT | |
| | GGTACTCCTCGCGCAATCAAATGGAAATCTTCAC | |
| | ACACCTGAACATCAAGCCTAAGAAGATCAACCT | |
| | GACGGCAGCTAATAAAATCCCGAAAGCCATGAT | |
| | TGATGAGTTCATCTTAAGCCCTGTAGTGAAACGT | |
| | ACATTCGGCCAAGCTATTAACTTAATCAACAAGA | |
| | TCATCGAGAAATACGGAGTCCCCGAAGATATCA | |
| | TCATCGAGCTGGCTCGCGAGAACAATTCCAAGG | |
| | ACAAGCAGAAGTTTATTAATGAGATGCAGAAAA | |
| | AGAACGAAAACACCCGCAAGCGCATTAACGAAA | |
| | TTATCGGTAAATACGGAAACCAAAATGCCAAGC | |
| | GTCTTGTGGAGAAGATTCGCCTGCATGATGAACA | |
| | GGAGGGTAAATGTCTGTATTCGTTGGAGTCTATT | |
| | CCTTTAGAGGATTTGCTGAATAACCCAAATCATT | |
| | ATGAAGTAGATCACATCATCCCGCGTTCCGTATC | |
| | ATTTGACAACAGTTACCATAACAAGGTGCTTGTT | |
| | AAGCAGTCGGAAGCAAGCAAGAAAAGTAATCTT | |
| | ACACCCTACCAATACTTCAATTCAGGCAAGTCAA | |
| | AACTGAGCTATAACCAATTTAAACAGCACATCCT | |
| | TAACCTTTCAAAGAGCCAGGATCGTATTTCTAAA | |
| | AAGAAGAAGGAATATCTTCTTGAGGAGCGTGAT | |
| | ATTAATAAGTTCGAGGTCCAGAAAGAATTCATTA | |
| | ATCGTAACTTAGTAGACACGCGCTACGCTACCCG | |
| | TGAATTAACAAATTACTTGAAAGCGTACTTCTCT | |
| | GCAAACAATATGAACGTTAAGGTGAAAACGATT | |
| | AATGGGTCCTTTACAGATTATTTGCGCAAAGTGT | |
| | GGAAGTTTAAGAAGGAACGCAACCACGGATACA | |
| | AACACCACGCGGAAGACGCTTTAATCATCGCCA | |
| | ATGCAGACTTCTTATTCAAGGAGAACAAGAAATT | |
| | AAAAGCGGTTAATTCAGTGCTTGAAAAGCCCGA | |
| | GATCGAATCGAAGCAGTTAGACATCCAAGTTGA | |
| | CTCCGAGGATAACTACTCTGAAATGTTTATTATC | |
| | CCGAAGCAGGTACAGGACATTAAGGACTTCCGT | |
| | AATTTCAAATATTCCCACCGTGTCGATAAGAAAC | |
| | CAAACCGTCAATTAATTAACGATACATTATACAG | |
| | CACCCGTAAGAAAGACAACTCAACTTACATCGT | |
| | GCAAACCATCAAGGACATCTATGCAAAGGATAA | |
| | CACAACACTGAAGAAACAGTTCGACAAGAGTCC | |
| | GGAGAAATTTCTGATGTACCAACACGACCCGCGT | |
| | ACCTTCGAGAAGTTAGAAGTCATCATGAAGCAA | |
| | TATGCTAATGAAAAAAATCCTCTTGCCAAGTATC | |
| | ACGAAGAAACGGGAGAATATCTGACCAAATACT | |
| | CCAAGAAAAACAATGGACCAATCGTTAAGAGCC | |
| | TGAAATACATTGGGAATAAGTTAGGCTCCCACTT | |
| | GGATGTTACGCATCAATTTAAAAGTTCCACTAAA | |
| | AAGCTTGTGAAGTTAAGTATTAAACCATACCGTT | |
| | TTGATGTATATTTGACAGATAAGGGTTACAAGTT | |
| | TATTACAATTAGTTACTTGGACGTTTTAAAAAAA | |
| | GACAACTACTACTACATCCCAGAGCAGAAATAT | |
| | GATAAATTAAAATTGGGTAAAGCGATTGATAAG | |
| | AATGCTAAGTTTATCGCCAGTTTCTATAAAAATG | |
| | ACCTGATCAAGCTGGACGGGGAGATTTACAAAA | |
| | TCATCGGGGTTAACTCCGACACACGCAACATGAT | |
| | CGAGCTTGACTTACCCGATATTCGTTACAAGGAG | |
| | TATTGCGAACTTAACAATATTAAGGGGGAACCTC | |
| | GTATTAAAAAGACAATCGGCAAAAAAGTTAATA | |
| | GTATTGAAAAACTGACAACCGATGTATTGGGCA | |
| | ACGTCTTTACAAACACCCAGTACACTAAACCTCA | |
| | GCTTCTTTTTAAACGCGGTAATAGCGGCGGTTCA | |
| | ATGACAAATCTGTCGGATATCATCGAAAAAGAA | |
| | ACCGGTAAACAGCTGGTTATCCAAGAAAGTATTT | |
| | TAATGCTTCCCGAAGAAGTCGAAGAGGTGATTG | |
| | GCAACAAACCCGAAAGTGATATTCTCGTGCATAC | |
| | TGCGTATGATGAGTCGACCGATGAAAACGTCAT | |
| | GCTGCTGACCTCCGACGCGCCCGAGTATAAACCG | |
| | TGGGCTTTGGTTATCCAGGATAGCAACGGTGAAA | |
| | ATAAGATTAAAATGTTATAA | |
| SaCas9- | TTGACAGCTAGCTCAGTCCTAGGTATAATACTAG | SEQ ID NO: 2 |
| sgRNA | TGTCCACCCGCAAAATTAAAAGTTTTAGTACTCT | |
| | GGAAACAGAATCTACTAAAACAAGGCAAAATGC | |
| | CGTGTTTATCTCGTCAACTTGTTGGCGAGATTTTT | |
| | ATGAGCAAAGGAGAAGAACTTTTCACTGGAGTT | |
| | GTCCCAATTCTTGTTGAATTAGATGGTGATGTTA | |
| | ATGGGCACAAATTTTCTGTCCGTGGAGAGGGTGA | |
| | AGGTGATGCTACAAACGGAAAACTCACCCTTAA | |
| | ATTTATTTGCACTACTGGAAAACTACCTGTTCCG | |
| | TGGCCAACACTTGTCACTACTCTGACCTATGGTG | |
| | TTCAATGCTTTTCCCGTTATCCGGATCACATGAA | |
| | ACGGCATGACTTTTTCAAGAGTGCCATGCCCGAA | |
| | GGTTATGTACAGGAACGCACTATATCTTTCAAAG | |
| | ATGACGGGACCTACAAGACGCGTGCTGAAGTCA | |
| | AGTTTGAAGGTGATACCCTTGTTAATCGTATCGA | |
| sfGFP-target- | GTTAAAGGGTATTGATTTTAAAGAAGATGGAAA | SEQ ID NO: 3 |
| degron | CATTCTTGGACACAAACTCGAGTACAACTTTAAC | |
| | TCACACAATGTATACATCACGGCAGACAAACAA | |
| | AAGAATGGAATCAAAGCTAACTTCAAAATTCGC | |
| | CACAACGTTGAAGATGGTTCCGTTCAACTAGCAG | |
| | ACCATTATCAACAAAATACTCCAATTGGCGATGG | |
| | CCCTGTCCTTTTACCAGACAACCATTACCTGTCG | |
| | ACACAATCTGTCCTTTCGAAAGATCCCAACGAAA | |
| | AGCGTGACCACATGGTCCTTCTTGAGTTTGTAAC | |
| | TGCTGCTGGGATTACACATGGCATGGATGAGCTC | |
| | TACAAATGGCCGCTTTTAATTTTACCCTGGAACG | |
| | CAGCGAACGACGAAAATTATGCGTTGGCAGCTT | |
| | AA | |
| NNGG PAM- | ATGGCGATTGGCCGCAAGATGTTTGAGTGATAAC | SEQ ID NO: 4 |
| target-sfGFP | TTAGCAAAGGAGAAGAACTTTTCACTGGAGTTGT | |
| | CCCAATTCTTGTTGAATTAGATGGTGATGTTAAT | |
| | GGGCACAAATTTTCTGTCCGTGGAGAGGGTGAA | |
| | GGTGATGCTACAAACGGAAAACTCACCCTTAAA | |
| | TTTATTTGCACTACTGGAAAACTACCTGTTCCGT | |
| | GGCCAACACTTGTCACTACTCTGACCTATGGTGT | |
| | TCAATGCTTTTCCCGTTATCCGGATCACATGAAA | |
| | CGGCATGACTTTTTCAAGAGTGCCATGCCCGAAG | |
| | GTTATGTACAGGAACGCACTATATCTTTCAAAGA | |
| | TGACGGGACCTACAAGACGCGTGCTGAAGTCAA | |
| | GTTTGAAGGTGATACCCTTGTTAATCGTATCGAG | |
| | TTAAAGGGTATTGATTTTAAAGAAGATGGAAAC | |
| | ATTCTTGGACACAAACTCGAGTACAACTTTAACT | |
| | CACACAATGTATACATCACGGCAGACAAACAAA | |
| | AGAATGGAATCAAAGCTAACTTCAAAATTCGCC | |
| | ACAACGTTGAAGATGGTTCCGTTCAACTAGCAGA | |
| | CCATTATCAACAAAATACTCCAATTGGCGATGGC | |
| | CCTGTCCTTTTACCAGACAACCATTACCTGTCGA | |
| | CACAATCTGTCCTTTCGAAAGATCCCAACGAAAA | |
| | GCGTGACCACATGGTCCTTCTTGAGTTTGTAACT | |
| | GCTGCTGGGATTACACATGGCATGGATGAGCTCT | |
| | ACAAATAA | |
| TadA8e- | ATGAGTGAGGTCGAATTTTCTCACGAATACTGGA | SEQ ID NO: 5 |
| SlugCas9 | TGCGCCACGCTCTTACTTTAGCCAAGCGTGCGCG | |
| | CGATGAGCGCGAGGTCCCGGTAGGAGCTGTCCT | |
| | GGTGTTAAACAACCGTGTGATCGGTGAAGGTTG | |
| | GAATCGTGCTATCGGTCTGCATGATCCAACGGCC | |
| | CATGCCGAGATCATGGCGCTTCGCCAGGGGGGG | |
| | CTGGTCATGCAAAACTACCGCCTTATTGATGCTA | |
| | CACTGTATGTCACTTTCGAGCCTTGTGTGATGTG | |
| | TGCGGGAGCCATGATCCACAGTCGTATTGGTCGT | |
| | GTCGTTTTTGGTGTACGTAACAGTAAACGCGGCG | |
| | CAGCTGGATCCTTAATGAACGTCTTAAATTATCC | |
| | AGGCATGAATCATCGTGTTGAGATTACTGAAGGC | |
| | ATCTTAGCGGATGAATGTGCCGCCCTTTTGTGCG | |
| | ATTTTTACCGTATGCCTCGCCAAGTGTTTAACGC | |
| | ACAGAAAAAGGCGCAATCGTCGATCAATTCCGG | |
| | TAGCGAAACACCGGGGACTTCAGAATCGGCCAC | |
| | CCCGGAGTCTATGAATCAAAAATTCATTCTGGGG | |
| | TTGGCTATTGGAATCACGTCGGTGGGGTATGGCC | |
| | TTATTGACTATGAAACCAAGAACATCATTGACGC | |
| | GGGGGTCCGTTTGTTCCCCGAGGCCAATGTCGAA | |
| | AATAACGAGGGCCGTCGTAGTAAACGCGGCTCC | |
| | CGTCGTCTTAAACGCCGCCGCATCCACCGTCTTG | |
| | AGCGCGTGAAGAAGTTGCTGGAGGATTATAACC | |
| | TGCTTGATCAGAGCCAGATCCCGCAGTCTACCAA | |
| | TCCATATGCTATTCGTGTCAAAGGGCTTTCTGAG | |
| | GCATTATCCAAGGACGAGCTGGTGATTGCATTAC | |
| | TTCACATTGCTAAACGCCGCGGTATTCATAAAAT | |
| | CGATGTAATCGATAGTAACGACGATGTTGGTAAC | |
| | GAGCTTTCGACGAAGGAACAGTTGAACAAAAAC | |
| | TCGAAACTTTTGAAGGATAAGTTCGTTTGTCAGA | |
| | TCCAACTTGAACGTATGAATGAAGGTCAAGTCCG | |
| | TGGAGAAAAGAATCGTTTTAAAACTGCCGATATT | |
| | ATCAAGGAAATCATCCAATTGCTTAATGTTCAAA | |
| | AAAATTTCCACCAATTAGACGAGAATTTTATCAA | |
| | CAAATACATCGAACTGGTGGAGATGCGTCGTGA | |
| | ATATTTTGAAGGACCTGGAAAGGGCAGTCCTTAT | |
| | GGGTGGGAAGGGGACCCCAAGGCCTGGTACGAG | |
| | ACCTTAATGGGGCATTGTACTTATTTTCCAGATG | |
| | AATTACGCTCAGTAAAGTATGCGTATAGCGCGG | |
| | ACTTGTTCAACGCGTTAAATGATTTAAACAATCT | |
| | GGTTATCCAGCGCGACGGCTTATCAAAGTTGGAG | |
| | TACCACGAGAAATATCATATTATTGAAAATGTAT | |
| | TTAAGCAGAAGAAAAAACCGACCTTAAAACAAA | |
| | TTGCTAATGAAATTAACGTAAATCCAGAAGATAT | |
| | TAAGGGTTATCGCATCACAAAGAGTGGCAAACC | |
| | TCAGTTCACGGAGTTCAAATTATACCATGATTTA | |
| | AAGTCCGTTCTTTTTGACCAATCTATTTTGGAAA | |
| | ACGAAGATGTGCTTGATCAGATTGCTGAAATCTT | |
| | AACGATTTACCAAGATAAAGATTCAATTAAATCG | |
| | AAGTTAACTGAGTTGGATATTTTACTGAATGAAG | |
| | AAGATAAGGAGAACATCGCTCAACTGACCGGAT | |
| | ACACCGGTACTCATCGCTTATCCTTAAAGTGCAT | |
| | TCGCTTGGTGTTGGAAGAACAATGGTACTCCTCG | |
| | CGCAATCAAATGGAAATCTTCACACACCTGAAC | |
| | ATCAAGCCTAAGAAGATCAACCTGACGGCAGCT | |
| | AATAAAATCCCGAAAGCCATGATTGATGAGTTC | |
| | ATCTTAAGCCCTGTAGTGAAACGTACATTCGGCC | |
| | AAGCTATTAACTTAATCAACAAGATCATCGAGA | |
| | AATACGGAGTCCCCGAAGATATCATCATCGAGCT | |
| | GGCTCGCGAGAACAATTCCAAGGACAAGCAGAA | |
| | GTTTATTAATGAGATGCAGAAAAAGAACGAAAA | |
| | CACCCGCAAGCGCATTAACGAAATTATCGGTAA | |
| | ATACGGAAACCAAAATGCCAAGCGTCTTGTGGA | |
| | GAAGATTCGCCTGCATGATGAACAGGAGGGTAA | |
| | ATGTCTGTATTCGTTGGAGTCTATTCCTTTAGAG | |
| | GATTTGCTGAATAACCCAAATCATTATGAAGTAG | |
| | ATCACATCATCCCGCGTTCCGTATCATTTGACAA | |
| | CAGTTACCATAACAAGGTGCTTGTTAAGCAGTCG | |
| | GAAGCAAGCAAGAAAAGTAATCTTACACCCTAC | |
| | CAATACTTCAATTCAGGCAAGTCAAAACTGAGCT | |
| | ATAACCAATTTAAACAGCACATCCTTAACCTTTC | |
| | AAAGAGCCAGGATCGTATTTCTAAAAAGAAGAA | |
| | GGAATATCTTCTTGAGGAGCGTGATATTAATAAG | |
| | TTCGAGGTCCAGAAAGAATTCATTAATCGTAACT | |
| | TAGTAGACACGCGCTACGCTACCCGTGAATTAAC | |
| | AAATTACTTGAAAGCGTACTTCTCTGCAAACAAT | |
| | ATGAACGTTAAGGTGAAAACGATTAATGGGTCC | |
| | TTTACAGATTATTTGCGCAAAGTGTGGAAGTTTA | |
| | AGAAGGAACGCAACCACGGATACAAACACCACG | |
| | CGGAAGACGCTTTAATCATCGCCAATGCAGACTT | |
| | CTTATTCAAGGAGAACAAGAAATTAAAAGCGGT | |
| | TAATTCAGTGCTTGAAAAGCCCGAGATCGAATCG | |
| | AAGCAGTTAGACATCCAAGTTGACTCCGAGGAT | |
| | AACTACTCTGAAATGTTTATTATCCCGAAGCAGG | |
| | TACAGGACATTAAGGACTTCCGTAATTTCAAATA | |
| | TTCCCACCGTGTCGATAAGAAACCAAACCGTCAA | |
| | TTAATTAACGATACATTATACAGCACCCGTAAGA | |
| | AAGACAACTCAACTTACATCGTGCAAACCATCA | |
| | AGGACATCTATGCAAAGGATAACACAACACTGA | |
| | AGAAACAGTTCGACAAGAGTCCGGAGAAATTTC | |
| | TGATGTACCAACACGACCCGCGTACCTTCGAGAA | |
| | GTTAGAAGTCATCATGAAGCAATATGCTAATGA | |
| | AAAAAATCCTCTTGCCAAGTATCACGAAGAAAC | |
| | GGGAGAATATCTGACCAAATACTCCAAGAAAAA | |
| | CAATGGACCAATCGTTAAGAGCCTGAAATACATT | |
| | GGGAATAAGTTAGGCTCCCACTTGGATGTTACGC | |
| | ATCAATTTAAAAGTTCCACTAAAAAGCTTGTGAA | |
| | GTTAAGTATTAAACCATACCGTTTTGATGTATAT | |
| | TTGACAGATAAGGGTTACAAGTTTATTACAATTA | |
| | GTTACTTGGACGTTTTAAAAAAAGACAACTACTA | |
| | CTACATCCCAGAGCAGAAATATGATAAATTAAA | |
| | ATTGGGTAAAGCGATTGATAAGAATGCTAAGTTT | |
| | ATCGCCAGTTTCTATAAAAATGACCTGATCAAGC | |
| | TGGACGGGGAGATTTACAAAATCATCGGGGTTA | |
| | ACTCCGACACACGCAACATGATCGAGCTTGACTT | |
| | ACCCGATATTCGTTACAAGGAGTATTGCGAACTT | |
| | AACAATATTAAGGGGGAACCTCGTATTAAAAAG | |
| | ACAATCGGCAAAAAAGTTAATAGTATTGAAAAA | |
| | CTGACAACCGATGTATTGGGCAACGTCTTTACAA | |
| | ACACCCAGTACACTAAACCTCAGCTTCTTTTTAA | |
| | ACGCGGTAATTAA | |
| MKIEE- | ATGAAAATCGAAGAAAATCAAAAATTCATTCTG | SEQ ID NO: 6 |
| SlugCas9- | GGGTTGGCTATTGGAATCACGTCGGTGGGGTATG | |
| 6xHis | GCCTTATTGACTATGAAACCAAGAACATCATTGA | |
| | CGCGGGGGTCCGTTTGTTCCCCGAGGCCAATGTC | |
| | GAAAATAACGAGGGCCGTCGTAGTAAACGCGGC | |
| | TCCCGTCGTCTTAAACGCCGCCGCATCCACCGTC | |
| | TTGAGCGCGTGAAGAAGTTGCTGGAGGATTATA | |
| | ACCTGCTTGATCAGAGCCAGATCCCGCAGTCTAC | |
| | CAATCCATATGCTATTCGTGTCAAAGGGCTTTCT | |
| | GAGGCATTATCCAAGGACGAGCTGGTGATTGCA | |
| | TTACTTCACATTGCTAAACGCCGCGGTATTCATA | |
| | AAATCGATGTAATCGATAGTAACGACGATGTTG | |
| | GTAACGAGCTTTCGACGAAGGAACAGTTGAACA | |
| | AAAACTCGAAACTTTTGAAGGATAAGTTCGTTTG | |
| | TCAGATCCAACTTGAACGTATGAATGAAGGTCA | |
| | AGTCCGTGGAGAAAAGAATCGTTTTAAAACTGC | |
| | CGATATTATCAAGGAAATCATCCAATTGCTTAAT | |
| | GTTCAAAAAAATTTCCACCAATTAGACGAGAATT | |
| | TTATCAACAAATACATCGAACTGGTGGAGATGC | |
| | GTCGTGAATATTTTGAAGGACCTGGAAAGGGCA | |
| | GTCCTTATGGGTGGGAAGGGGACCCCAAGGCCT | |
| | GGTACGAGACCTTAATGGGGCATTGTACTTATTT | |
| | TCCAGATGAATTACGCTCAGTAAAGTATGCGTAT | |
| | AGCGCGGACTTGTTCAACGCGTTAAATGATTTAA | |
| | ACAATCTGGTTATCCAGCGCGACGGCTTATCAAA | |
| | GTTGGAGTACCACGAGAAATATCATATTATTGAA | |
| | AATGTATTTAAGCAGAAGAAAAAACCGACCTTA | |
| | AAACAAATTGCTAATGAAATTAACGTAAATCCA | |
| | GAAGATATTAAGGGTTATCGCATCACAAAGAGT | |
| | GGCAAACCTCAGTTCACGGAGTTCAAATTATACC | |
| | ATGATTTAAAGTCCGTTCTTTTTGACCAATCTATT | |
| | TTGGAAAACGAAGATGTGCTTGATCAGATTGCTG | |
| | AAATCTTAACGATTTACCAAGATAAAGATTCAAT | |
| | TAAATCGAAGTTAACTGAGTTGGATATTTTACTG | |
| | AATGAAGAAGATAAGGAGAACATCGCTCAACTG | |
| | ACCGGATACACCGGTACTCATCGCTTATCCTTAA | |
| | AGTGCATTCGCTTGGTGTTGGAAGAACAATGGTA | |
| | CTCCTCGCGCAATCAAATGGAAATCTTCACACAC | |
| | CTGAACATCAAGCCTAAGAAGATCAACCTGACG | |
| | GCAGCTAATAAAATCCCGAAAGCCATGATTGAT | |
| | GAGTTCATCTTAAGCCCTGTAGTGAAACGTACAT | |
| | TCGGCCAAGCTATTAACTTAATCAACAAGATCAT | |
| | CGAGAAATACGGAGTCCCCGAAGATATCATCAT | |
| | CGAGCTGGCTCGCGAGAACAATTCCAAGGACAA | |
| | GCAGAAGTTTATTAATGAGATGCAGAAAAAGAA | |
| | CGAAAACACCCGCAAGCGCATTAACGAAATTAT | |
| | CGGTAAATACGGAAACCAAAATGCCAAGCGTCT | |
| | TGTGGAGAAGATTCGCCTGCATGATGAACAGGA | |
| | GGGTAAATGTCTGTATTCGTTGGAGTCTATTCCT | |
| | TTAGAGGATTTGCTGAATAACCCAAATCATTATG | |
| | AAGTAGATCACATCATCCCGCGTTCCGTATCATT | |
| | TGACAACAGTTACCATAACAAGGTGCTTGTTAAG | |
| | CAGTCGGAAGCAAGCAAGAAAAGTAATCTTACA | |
| | CCCTACCAATACTTCAATTCAGGCAAGTCAAAAC | |
| | TGAGCTATAACCAATTTAAACAGCACATCCTTAA | |
| | CCTTTCAAAGAGCCAGGATCGTATTTCTAAAAAG | |
| | AAGAAGGAATATCTTCTTGAGGAGCGTGATATTA | |
| | ATAAGTTCGAGGTCCAGAAAGAATTCATTAATCG | |
| | TAACTTAGTAGACACGCGCTACGCTACCCGTGAA | |
| | TTAACAAATTACTTGAAAGCGTACTTCTCTGCAA | |
| | ACAATATGAACGTTAAGGTGAAAACGATTAATG | |
| | GGTCCTTTACAGATTATTTGCGCAAAGTGTGGAA | |
| | GTTTAAGAAGGAACGCAACCACGGATACAAACA | |
| | CCACGCGGAAGACGCTTTAATCATCGCCAATGCA | |
| | GACTTCTTATTCAAGGAGAACAAGAAATTAAAA | |
| | GCGGTTAATTCAGTGCTTGAAAAGCCCGAGATCG | |
| | AATCGAAGCAGTTAGACATCCAAGTTGACTCCG | |
| | AGGATAACTACTCTGAAATGTTTATTATCCCGAA | |
| | GCAGGTACAGGACATTAAGGACTTCCGTAATTTC | |
| | AAATATTCCCACCGTGTCGATAAGAAACCAAAC | |
| | CGTCAATTAATTAACGATACATTATACAGCACCC | |
| | GTAAGAAAGACAACTCAACTTACATCGTGCAAA | |
| | CCATCAAGGACATCTATGCAAAGGATAACACAA | |
| | CACTGAAGAAACAGTTCGACAAGAGTCCGGAGA | |
| | AATTTCTGATGTACCAACACGACCCGCGTACCTT | |
| | CGAGAAGTTAGAAGTCATCATGAAGCAATATGC | |
| | TAATGAAAAAAATCCTCTTGCCAAGTATCACGAA | |
| | GAAACGGGAGAATATCTGACCAAATACTCCAAG | |
| | AAAAACAATGGACCAATCGTTAAGAGCCTGAAA | |
| | TACATTGGGAATAAGTTAGGCTCCCACTTGGATG | |
| | TTACGCATCAATTTAAAAGTTCCACTAAAAAGCT | |
| | TGTGAAGTTAAGTATTAAACCATACCGTTTTGAT | |
| | GTATATTTGACAGATAAGGGTTACAAGTTTATTA | |
| | CAATTAGTTACTTGGACGTTTTAAAAAAAGACAA | |
| | CTACTACTACATCCCAGAGCAGAAATATGATAA | |
| | ATTAAAATTGGGTAAAGCGATTGATAAGAATGC | |
| | TAAGTTTATCGCCAGTTTCTATAAAAATGACCTG | |
| | ATCAAGCTGGACGGGGAGATTTACAAAATCATC | |
| | GGGGTTAACTCCGACACACGCAACATGATCGAG | |
| | CTTGACTTACCCGATATTCGTTACAAGGAGTATT | |
| | GCGAACTTAACAATATTAAGGGGGAACCTCGTA | |
| | TTAAAAAGACAATCGGCAAAAAAGTTAATAGTA | |
| | TTGAAAAACTGACAACCGATGTATTGGGCAACG | |
| | TCTTTACAAACACCCAGTACACTAAACCTCAGCT | |
| | TCTTTTTAAACGCGGTAATGGGTCGGGCGGCGGC | |
| | GGCTCGGGCAAGCGTACAGCCGACGGAAGTGAG | |
| | TTCGAGCCTAAAAAGAAGCGTAAGGTACATCAC | |
| | CATCACCATCATTAA | |
| MbPyIRS | ATGGATAAGAAGCCGCTGGATGTTCTGATCTCTG | SEQ ID NO: 7 |
| (IPYE) | CGACCGGTCTGTGGATGTCCCGTACCGGCACGCT | |
| | GCACAAGATCAAGCACTATGAGATTTCTCGTTCT | |
| | AAAATCTACATCGAAATGGCGTGTGGTGACCATC | |
| | TGGTTGTGAACAACTCTCGTTCTTGTCGTCCCGC | |
| | ACGTGCATTCCGTTATCATAAATACCGTAAAACC | |
| | TGCAAACGTTGTCGTGTTTCTGACGAAGATATCA | |
| | ACAACTTCCTGACCCGTTCTACCGAAGGCAAAAC | |
| | CTCTGTTAAAGTTAAAGTTGTTTCTGAGCCGAAA | |
| | GTGAAAAAAGCGATGCCGAAATCTGTTTCTCGTG | |
| | CGCCGAAACCGCTGGAAAATCCGGTTTCTGCGA | |
| | AAGCGTCTACCGACACCTCTCGTTCTGTTCCGTC | |
| | TCCGGCGAAATCTACCCCGAACTCTCCGGTTCCG | |
| | ACCTCTGCGCCGGCGCCGTCTCTGACCCGTTCTC | |
| | AGCTGGATCGTGTTGAAGCGCTGCTGTCTCCGGA | |
| | AGATAAAATCTCTCTGAACATCGCGAAACCGTTC | |
| | CGTGAACTGGAATCTGAACTGGTTACCCGTCGTA | |
| | AAAACGATTTCCAGCGTCTGTACACCAACGATCG | |
| | TGAAGACTACCTGGGTAAACTGGAACGTGACAT | |
| | CACCAAATTCTTCGTTGACCGTGATTTCCTGGAA | |
| | ATCAAATCTCCGATCCTGATCCCGGCGGAATACG | |
| | TTGAACGTATGGGTATCAACAACGATACCGAACT | |
| | GTCTAAACAGATCTTCCGTGTTGATAAAAACCTG | |
| | TGCCTGCGTCCGATGCTGGCGCCGACCCTGTACA | |
| | ACTATCTGCGTAAACTGGATCGTATCCTGCCGGA | |
| | CCCGATCAAAATCTTCGAAGTTGGTCCGTGCTAC | |
| | CGTAAAGAATCTGACGGTAAAGAACACCTGGAA | |
| | GAGTTCACCATGGTGAACTTCTGCCAGATGGGTT | |
| | CTGGTTGCACCCGTGAGAACCTGGAATCTCTGAT | |
| | CAAAGAATTTCTGGACTACCTGGAAATCGACTTC | |
| | GAAATCGTTGGTGACTCCTGCATGGTGTACGGTG | |
| | ATACCCTGGACATCATGCACGGTGACCTGGAACT | |
| | GTCTTCTGCGGTTGTTGGTCCGGTTCCGCTGGAT | |
| | CGTGAATGGGGTATCGACAAACCGTGGATCGGT | |
| | GCGGGTTTCGGTCTGGAACGTCTGCTGAAAGTTA | |
| | TGCACGGTTTCAAAAACATCAAACGTGCGTCTCG | |
| | TTCTGAATCTTACTACAACGGTATCTCTACCAAC | |
| | CTGTAA | |
| MbPylT | TGTGCTTCTCAAATGCCTGAGGCCAGTTTGCTCA | SEQ ID NO: 8 |
| | GGCTCTCCCCGTGGAGGTAATAATTGACGATATG | |
| | ATCAGTGCACGGCTAACTAAGCGGCCTGCTGACT | |
| | TTCTCGCCGATCAAAAGGCATTTTGCTATTAAGG | |
| | GATTGACGAGGGCGTATCTGCGCAGTAAGATGC | |
| | GCCCCGCATTCGGAAACGTGATCATGTAGATCGA | |
| | AtGGACTCTAAATCCGTTCAGTGGGGTTAGATTCC | |
| | CCACGTTTCCGCCA | |

### EXAMPLES

### Example 1

### Methods

A critical challenge hindering the development of a general artificial intelligence model for protein function prediction is the scarcity of large-scale datasets linking enzyme sequences to their functions. Due to the limitations in experimental data collection, there is currently no other effective experimental method to generate comprehensive enzyme sequence-function datasets. Consequently, existing datasets for enzyme sequence-function relationships are limited in both scope and diversity. Fewer than twenty datasets are commonly utilized for training machine learning models in protein function prediction, and most of these contain fewer than 10,000 data points. This stands in sharp contrast to the datasets used to train advanced machine learning models like AlphaFold, which leverage over 200 million sequence-structure data points.

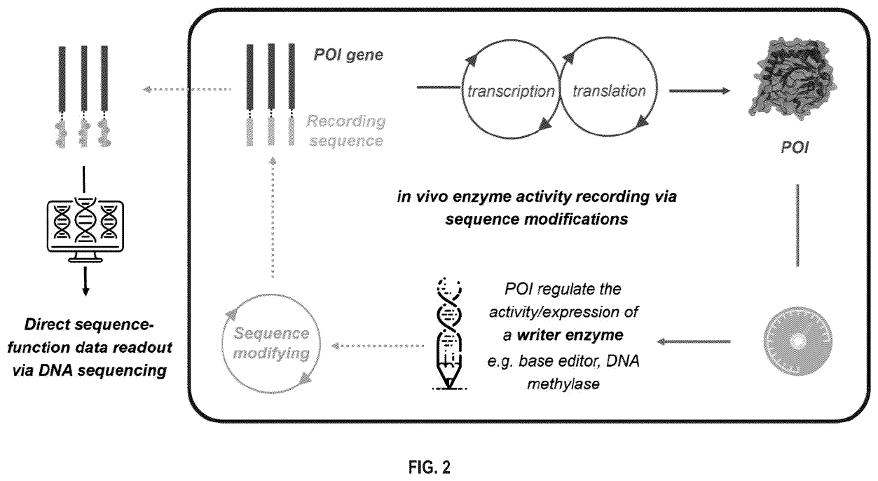

This study introduces an innovative technique termed “sequence display,” which offers an efficient and high-throughput platform for generating large-scale enzyme sequence-function datasets (FIG. 2). The core principle of the sequence display platform is as follows: First, a recording sequence is attached to the gene encoding the protein of interest (POI). Second, the function of the POI is linked to the activity of a writer enzyme, which induces sequence modifications in the recording sequence. These modifications serve as a direct reflection of the POI's activity. By employing high-throughput sequencing technologies, such as Illumina or Oxford Nanopore sequencing, the sequence of the POI and the modifications in the recording sequence can be simultaneously read. This enables the direct correlation of the POI's sequence with its function, allowing sequence-function data to be obtained in a single sequencing run.

This approach eliminates the labor-intensive library preparation and screening processes required by traditional methods, such as fluorescence-activated cell sorting (FACS). Moreover, compared to existing techniques like FACS-based or growth-based methods, sequence display delivers more accurate and high-resolution sequence-function data, with significantly improved dynamic range and precision.

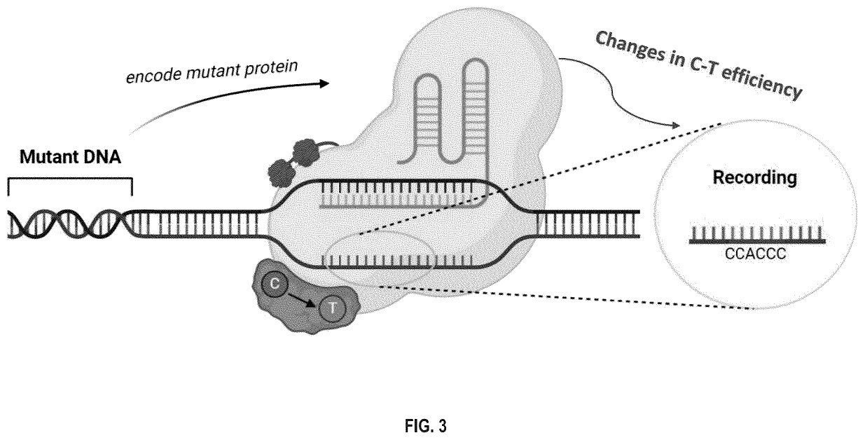

As a proof of concept, this platform was initially applied to generate a sequence-function dataset for the Uracil DNA Glycosylase Inhibitor (UGI) domain of a cytosine base editor, which plays a critical role in preventing the conversion of uracil into an apurinic/apyrimidinic (AP) site. The foundational design is illustrated in the figure below (FIG. 3). A recording sequence containing multiple cytosines (e.g., a “CCACCC” motif) was inserted downstream of the stop codon of the base editor, adjacent to the UGI domain. Mutations introduced in the UGI domain alter the cytosine-to-thymine (C-T) editing efficiency of the base editor, which subsequently causes variations in the C-T mutation frequencies within the CCACCC region of the recording sequence. This approach enables the direct correlation of UGI domain mutations with their functional impact on editing efficiency, as reflected in the recording sequence.

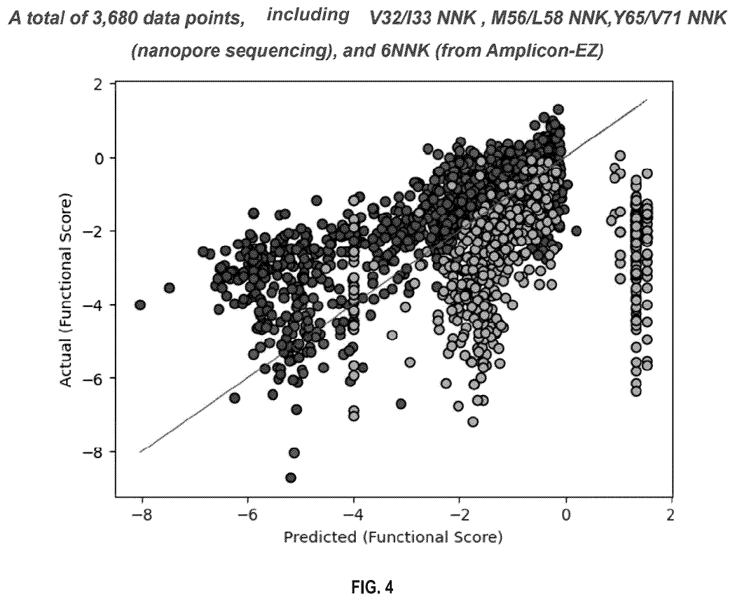

Using this strategy, a small library dataset was generated including 3,680 data points for double-site saturation NNK libraries at positions V32/I33, M56/L58, and Y65/V71, as well as a subset of multi-site saturation libraries covering all six sites (FIG. 4). This sequence-function dataset was subsequently used to train a machine learning model, which successfully captured the sequence-function relationship of the UGI domain within the base editor. The model's effectiveness is demonstrated by the strong linear correlation between the experimentally measured activity data and the activity predictions generated by the machine learning model.

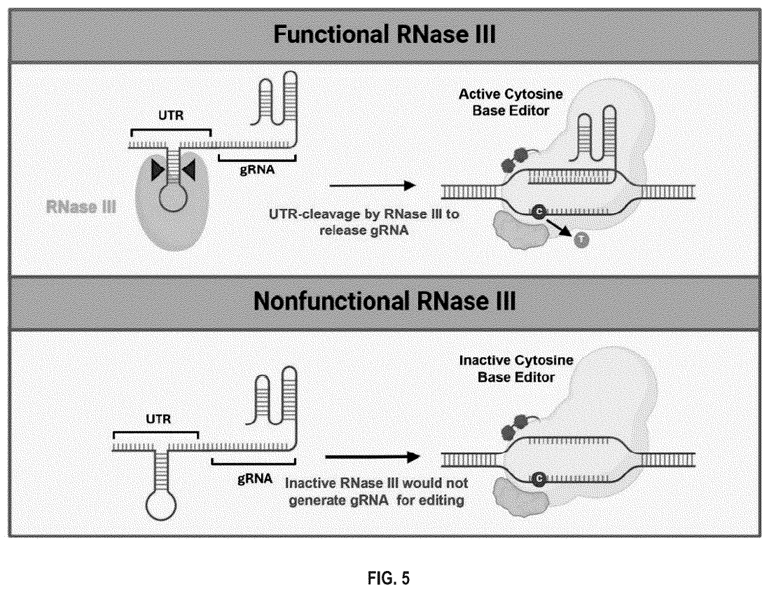

The Sequence Display platform is not limited to profiling sequence-function datasets for domains of base editors. In principle, by designing appropriate genetic circuits, any protein whose activity can be linked to the regulation of a writer enzyme can be profiled using this technique to generate sequence-function datasets. To demonstrate this potential, Sequence Display was next applied to obtain a sequence-function dataset for RNase III. The design is as follows: the gRNA essential for the activity of a base editor was fused to an untranslated region (UTR). RNase III cleaves this UTR, enabling the formation of functional gRNA (FIG. 5) In this way, the activity of RNase III is linked to the activity regulation of the base editor. Ostermeier et al. previously used a similar design to study the sequence-function relationship of RNase III using fluorescence-activated cell sorting (FACS), generating sequence-function data for single-site saturation mutagenesis libraries of all RNase III residues.

To demonstrate the applicability of Sequence Display for RNase III, ten representative RNase III mutants characterized by Ostermeier et al. were selected, which cover a wide range of functional regions and features, and a library was constructed using the platform outlined herein to profile their sequence-function data using Sequence Display. Results showed that Sequence Display accurately recapitulated the relative activities of these ten mutants, closely matching the relative activities reported by the Ostermeier group (Table 1). These findings highlight the broad applicability of the Sequence Display technique. Much larger-scale sequence-function datasets may be generated particularly for multi-site combinatorial site-saturation mutagenesis libraries of RNase III.

TABLE 1
Initial results revealed a strong correlation between
the RNase III mutant activity obtained via Sequence
Display and the activity previously determined by Ostermeier
et al. through biochemical characterization.

| | | | | | Functional |
|---|---|---|---|---|---|
| | | Functional | | | Score |
| | | Score | | | Sequence |
| Variant | Fitness | Mean | Replica 1 | Replica 2 | Display |
| Wild Type | 1.00 ± 0.01 | 0 | 0 | 0 | 0 |
| E65P | 1.00 ± 0.01 | −0.09 | 0.04 + | −0.13 + | −0.55 |
| | | | 0.02/−0.02 | 0.01/−0.01 | |
| D114G | 0.99 ± 0.01 | −0.04 | 0.07 + | −0.07 + | −0.44 |
| | | | 0.03/−0.04 | 0.01/−0.01 | |
| E38Q | 0.99 ± 0.01 | −0.11 | 0.01 + | −0.05 + | −0.73 |
| | | | 0.02/−0.02 | 0.01/−0.01 | |
| D114R | 0.97 ± 0.01 | −0.02 | 0.01 + | −0.03 + | −1.05 |
| | | | 0.05/−0.05 | 0.01/−0.01 | |
| D155E | 0.90 ± 0.01 | −0.36 | −0.09 + | −0.45 + | −0.64 |
| | | | 0.04/−0.05 | 0.05/−0.05 | |
| E38A | 0.88 ± 0.01 | −1.47 | −1.51 + | −0.46 + | −0.85 |
| | | | 0.03/−0.03 | 0.04/−0.04 | |
| F188D | 0.83 ± 0.01 | −0.13 | −0.017 + | 0.01 + | −2.17 |
| | | | 0.07/−0.08 | 0.04/−0.04 | |
| E38V | 0.80 ± 0.01 | −1.55 | −1.58 + | −0.65 + | −2.10 |
| | | | 0.02/−0.02 | 0.05/−0.06 | |
| C192C | 0.80 ± 0.01 | −0.91 | −0.96 + | −0.63 + | −2.88 |
| | | | 0.06/−0.07 | 0.16/−0.25 | |
| TCT | 0.76 ± 0.01 | −2.33 | −2.40 + | −0.88 + | −2.27 |
| | | | 0.15/−0.23 | 0.20/−0.38 | |
| ΔdsRBD⁸ | 0.75 ± 0.01 | n.d. | n.d. | n.d. | n.d. |
| E117K | 0.71 ± 0.01 | −2.18 | −2.20 + | −0.84 + | −2.26 |
| | | | 0.01/−0.01 | 0.01/−0.01 | |

### Design of the SDEML Platform for SlugCas9 Evolution Towards Diverse PAM Specificities

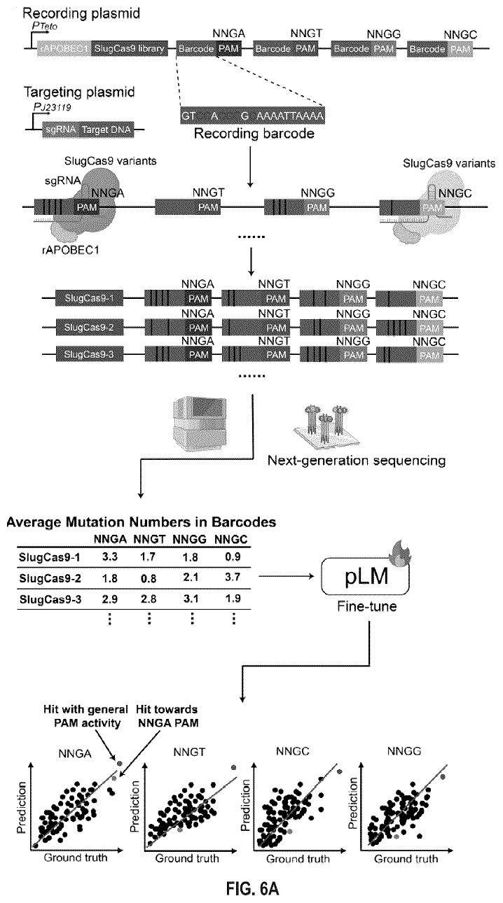

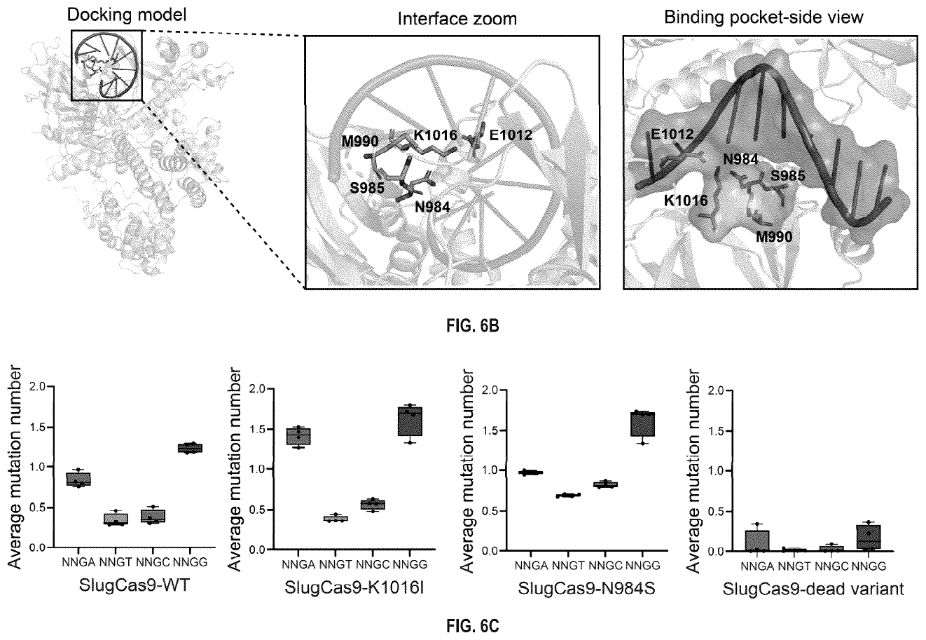

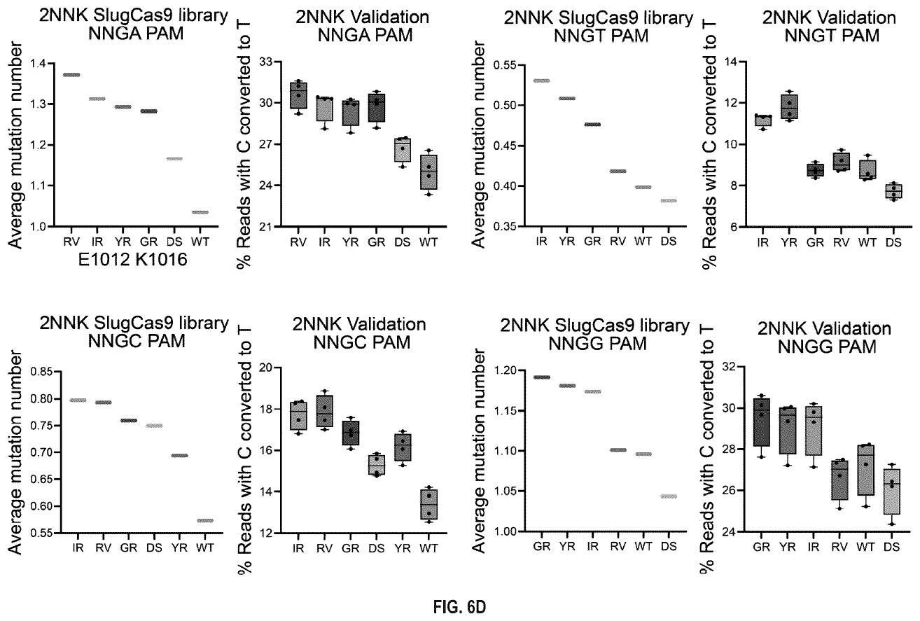

Compared to the widely used SpCas9 and its variants, which are too large to be delivered via a single adeno-associated virus (AAV) for in-vivo therapies, SlugCas9 represents a compact Cas9 nuclease that can be efficiently packaged into a single AAV. This feature positions SlugCas9 as a promising tool for therapeutic applications. However, the limited PAM recognition of compact Cas9 nucleases poses a challenge for their use in gene editing applications. For example, SlugCas9-WT specifically recognizes the NNGG PAM, which is more restrictive compared to the broader NNG PAM recognized by SpCas9. One goal of the systems outlined herein was to engineer SlugCas9 variants capable of recognizing a broader NNG PAM with enhanced efficiency. First, the SDEML pipeline was established to engineer SlugCas9 variants tailored for specific PAM requirements (FIG. 6A). A cytosine deaminase (rAPOBEC1) and a SlugCas9 library were incorporated into a recording plasmid including four recording barcodes with different PAM sequences (NNGA, NNGT, NNGC, NNGG) downstream of the SlugCas9 gene. These recording barcodes are 20 bp DNA sequences with six cytosine bases, which can mutate to thymine (T) bases upon the action of rAPOBEC1. Another writing plasmid included SaCas9-sgRNA, which can accurately guide SlugCas9 to the barcodes and uses the rAPOBEC1 to introduce mutations in barcodes. If certain SlugCas9 variants exhibit strong bias toward the NNGA PAM, rAPOBEC1 will introduce more mutations in the recording barcodes located near NNGA PAMs. Conversely, SlugCas9 variants with low activity toward the NNGC PAM will result in fewer mutations in the corresponding recording barcodes. After the barcode hyper-mutagenesis reaction, a SlugCas9 barcode-mutated library was obtained, enabling the activity of each SlugCas9 variant to be assessed based on the mutations in the barcodes. By using NGS to sequence both the SlugCas9 variants and their corresponding recording barcodes near each PAM, the average number of mutations in the barcodes was determined for each variant toward each PAM. These large sequence-activity datasets were then utilized for transfer learning using pre-trained pLMs to train a model that maps SlugCas9 variant sequences to their activities. The trained model was subsequently used to predict the activities of different SlugCas9 variants, enabling the identification of both general hits capable of recognizing the broad NNG PAM and specific hits with selectivity for one or two PAMs.

Next, AlphaFold2 was used to predict the 3D structure of SlugCas9 and dock it with the recording barcode and NNGG PAM. Based on the predicted structure, N984, M990, 5985, E1012, and K1016 were identified as being near the Cas9 binding sites and the PAM motif (FIG. 6B). It is hypothesized that mutating these residues will alter the recognition bias of the SlugCas9 protein, potentially enhancing its binding affinity for diverse PAM sequences.

The functions performed in the processes and methods may be implemented in differing order. Furthermore, the outlined steps and operations are provided as examples, and some of the steps and operations may be optional, combined into fewer steps and operations, or expanded into additional steps and operations without detracting from the essence of the disclosed embodiments.

### Evaluation of the Accuracy of the Sequence Display Evolution System for SlugCas9 Engineering

Previously, the activities of SlugCas9 and its variants toward different PAMs were reported. To evaluate the system outlined herein, SlugCas9-WT and these reported variants were constructed and subjected to SD hypermutagenesis reactions. Sequencing results revealed that SlugCas9-WT exhibited average mutation numbers in the order of NNGG>NNGA>NNGC>NNGT, consistent with the activities reported in prior studies (FIG. 6C). Additionally, the SlugCas9 variants SlugCas9-N984S and SlugCas9-K1016I demonstrated higher activities across all four PAMs compared to SlugCas9-WT, aligning with previous findings. As expected, the catalytically inactive SlugCas9 (dead variant) showed minimal activity across all PAMs, confirming the low background signal of the SD system.

Structural docking analysis identified residues N984, M990, S985, E1012, and K1016 as potentially critical for PAM recognition and binding. Among these, E1012 and K1016 were selected to construct a focused 2NNK library (N=any nucleotide, K=G or T), as these residues have also been reported in previous studies to play important roles in SlugCas9 activity. The 2NNK library plasmids, along with writing plasmids, were transformed into E. coli, and SD hypermutagenesis reactions were performed. NGS was then employed to sequence the four barcodes and the 2NNK sites in SlugCas9 variants, generating data that linked the activities of different variants to their respective PAM preferences. Six 2NNK SlugCas9 variants were selected at random for validation. Comparing the average mutation numbers of different variants against the same PAM, the overall trend from the library experimental data is consistent with the validation data, with only minor discrepancies (FIG. 6D). These results demonstrate that the SD evolution system outlined herein can correlate protein activities with their genotype sequences and provide enough resolution and sufficient accuracy to meet the requirements of machine learning.

### Construction of a 5NNK SlugCas9 Library for Sequence Display Evolution

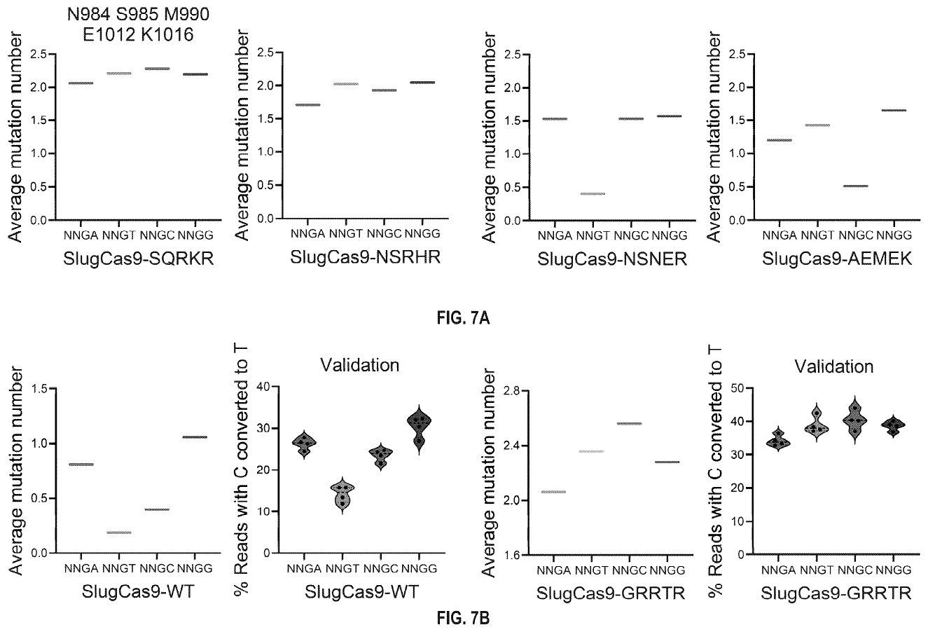

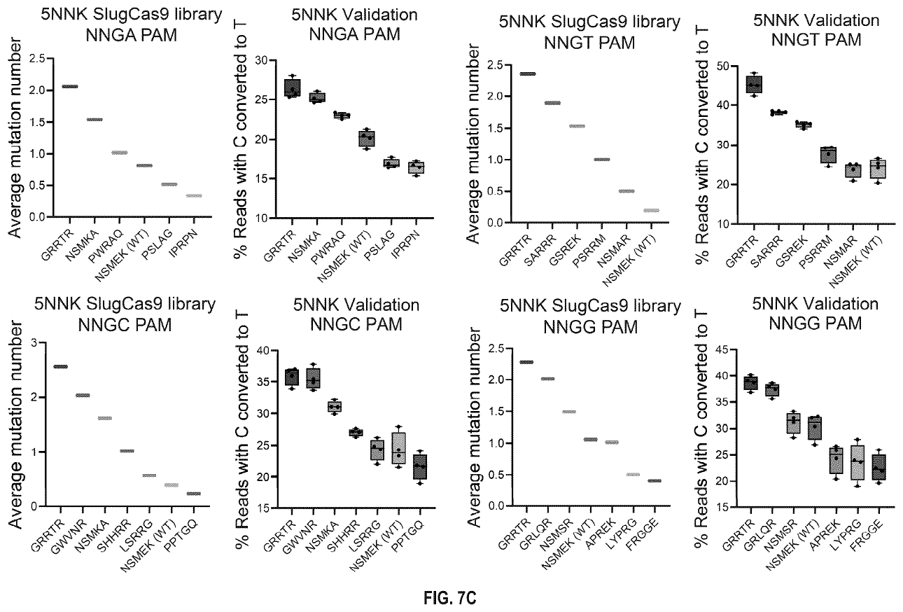

Focusing on five important residues based on the docking results (FIG. 6B), a focused 5NNK library of SlugCas9 was created to facilitate SDEML-driven evolution of SlugCas9. Each of these residues in SlugCas9 was subjected to site-saturation mutagenesis, randomized to NNK codons (N=any nucleotide, K=G or T), enabling coverage of all 20 amino acids. Over 105 SlugCas9 variants underwent SD hypermutagenesis reactions. NGS was used to analyze the SlugCas9 variants and the corresponding mutations in four recording barcodes, each located near a different PAM. This process generated a large sequence-activity dataset for SlugCas9 variants. From the dataset, approximately 1.1×105 variants had more than 10 reads, ~3×104 variants had more than 50 reads, ~16,000 variants had more than 100 reads, and ~11,000 variants had more than 200 reads. These results confirm that the dataset provides sufficient and accurate sequence-activity data to train machine learning models, enabling the discovery of underlying relationships between SlugCas9 variants and their preferences for different PAMs. Analysis of the NGS results identified promising SlugCas9 variants capable of recognizing all four PAMs with comparable activity levels, including SlugCas9-SQRKR and SlugCas9-NSRHR (FIG. 7A). Additionally, specific variants were discovered that selectively recognized three PAMs while exhibiting low activity toward an alternative PAM motif, such as SlugCas9-NSNER and SlugCas9-AEMEK.

For the SlugCas9-WT, the large library sequence data and NGS validation data both showed that the average mutation numbers follow the order NNGG>NNGA>NNGC>NNGT, consistent with results above and the reported activities of SlugCas9 in previous studies (FIG. 7B). Based on the results from all sequenced variants and NGS validation experiments, SlugCas9-GRRTR exhibited the highest activity across all four PAM modes. To evaluate the accuracy and resolution of the SDEML system for SlugCas9, distinct SlugCas9 variants with different activities toward the four PAMs were selected for NGS validation. A comparison between the large library sequencing data and the validation results demonstrated a strong correlation in activity levels for each variant in four PAM modes (FIG. 7C). These results demonstrate that the SD evolution system can generate a comprehensive and high-quality dataset linking SlugCas9 variants to their activities for PAM recognition, providing a robust foundation for downstream machine learning to model these relationships.

### Statistical Analysis of Appropriate Sample Size for Detecting Differences of Average Mutation Numbers Among SlugCas9 Variants

Statistical analysis was performed on the SlugCas9 variant results from the 2NNK NGS validation dataset to evaluate the resolution of the SDEML system for SlugCas9 evolution. The weighted average population variance of the samples from the 2NNK NGS validation data was used to calculate the appropriate sample size required to detect differences in the average mutation numbers among SlugCas9 variants. The appropriate number of reads required to detect differences in average mutation numbers were evaluated under varying confidence levels and test power (Table 2). As the confidence level increases from 80% to 95%, the likelihood of false-positive results decreases, but the number of reads required for detecting differences in each variant increases. Similarly, increasing the power of a test from 60% to 80% reduces the probability of false-negative results, also requiring a higher number of reads. For new technology evaluations, such as SDEML, a confidence level of 90% and a test power of 60% are generally acceptable for initial testing and technology development. Under these conditions, approximately 20 reads are sufficient to detect a difference of 1 in average mutation numbers among SlugCas9 variants. For detecting smaller differences, the required reads increase: ~50 reads for a 0.5 difference, ~100 reads for a 0.3 difference, and ~1,000 reads for a 0.1 difference. These results highlight the relationship between resolution and the number of reads needed for precise differentiation among SlugCas9 variants.

To balance resolution and the number of variants for training ML models, a filtering strategy was applied to exclude 5NNK SlugCas9 variants with fewer than 100 reads. This approach ensures high-resolution data while retaining a substantial dataset (~16,000 variants) for model training, validation, and testing.

Appendix A details more information about determination of sample size for detecting differences between variants, and is incorporated by reference in its entirety.

### Modeling the Relationship Between SlugCas9 Variants and their Activities Using pLM

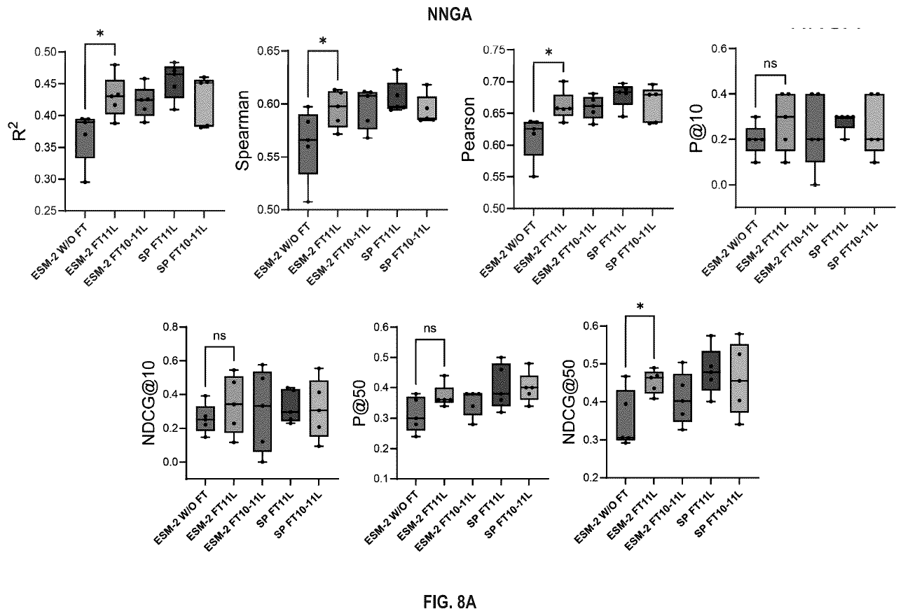

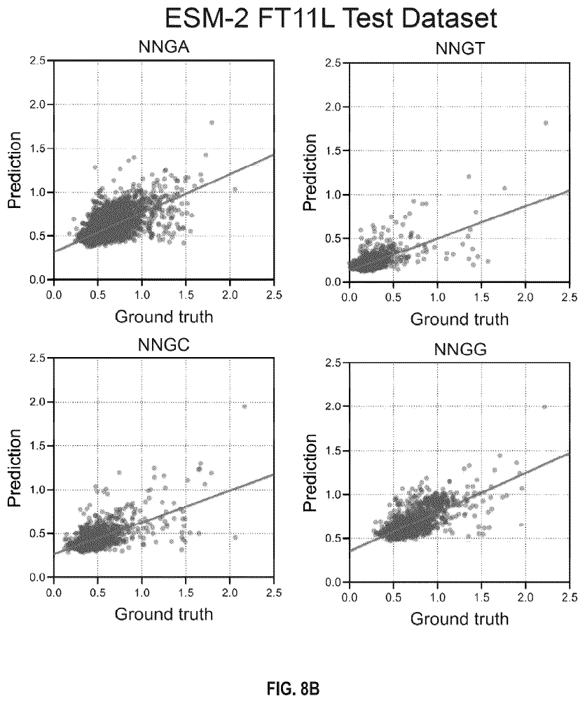

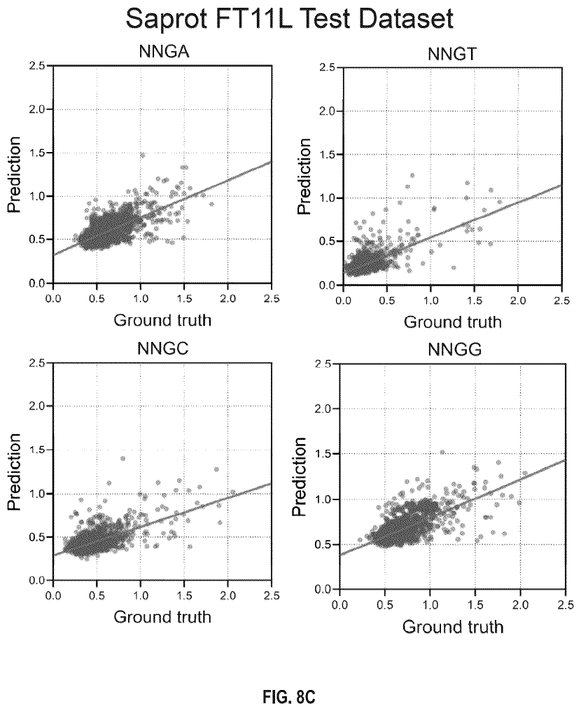

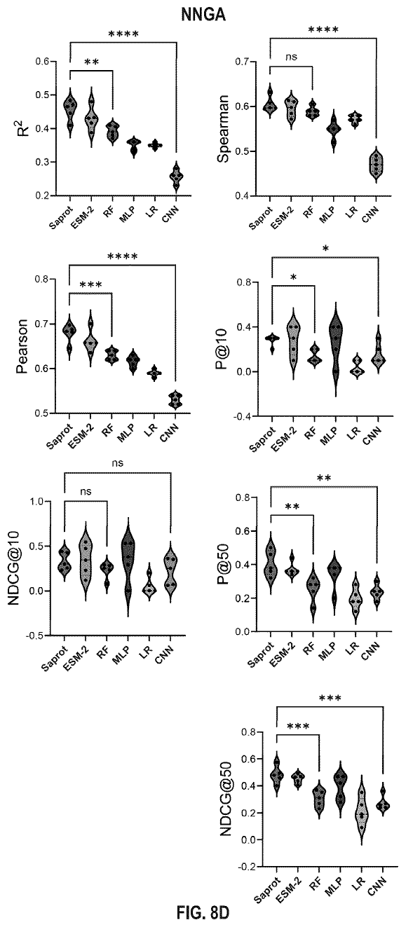

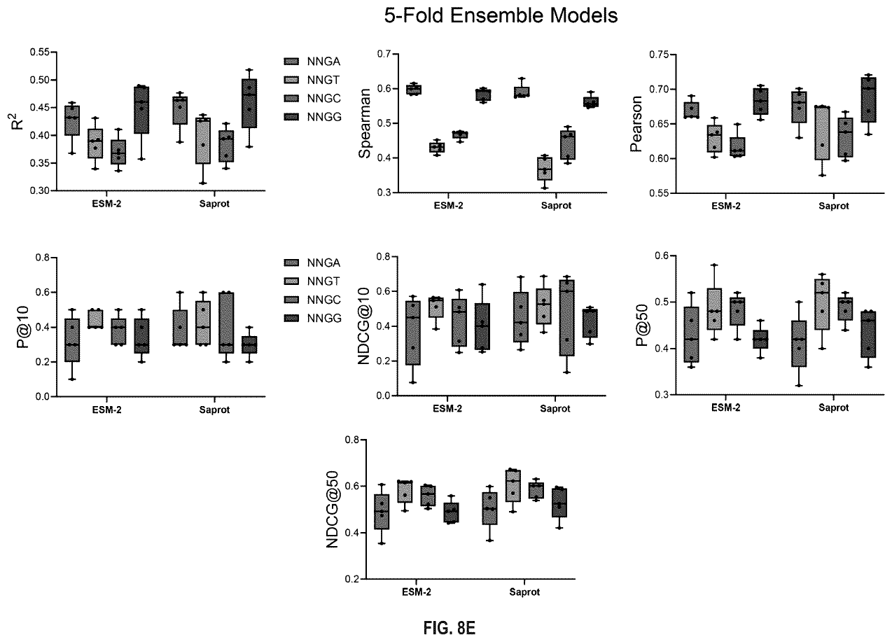

After validating the accuracy of the SDEML system for 5NNK SlugCas9, ESM-2 (35M) and SaProt (35M) were used to extract embedding dimensions (Emb Dim) for different SlugCas9 variants. Different fine-tuning strategies for ESM-2 and Saprot models were compared and their performances were evaluated using seven distinct metrics (FIG. 8A). Metrics such as R², Spearman's rank correlation coefficient, and Pearson's correlation coefficient were employed to assess the overall prediction accuracy across the entire test dataset. These metrics provide insight into the model's ability to capture the relationship between SlugCas9 variant sequences and their activities comprehensively.

TABLE 2
Required sample size to detect differences in average
mutation number for different PAMs across variants.

| Confidence | Power of a | Hypothesized | Sample size range for different PAM | | | |
|---|---|---|---|---|---|---|
| level (%) | test (%) | difference | NNGA | NNGT | NNGC | NNGG |
| 80 | 60 | 1 | 8 | 8 | 13-14 | 9 |
| | 70 | | 11 | 11-12 | 18-19 | 12 |
| | 80 | | 15 | 15-16 | 25-26 | 16-17 |
| | 60 | 0.5 | 30-32 | 31-32 | 51-54 | 33-35 |
| | 70 | | 42-44 | 43-45 | 70-74 | 46-48 |
| | 80 | | 58-60 | 59-61 | 97-102 | 63-66 |
| | 60 | 0.3 | 84-87 | 86-89 | 141-148 | 92-96 |
| | 70 | | 115-121 | 119-123 | 195-205 | 127-133 |
| | 80 | | 159-167 | 164-170 | 269-283 | 175-184 |
| | 60 | 0.1 | 748-782 | 770-796 | 1262-1327 | 820-862 |
| | 70 | | 1035-1083 | 1066-1102 | 1747-1837 | 1136-1193 |
| | 80 | | 1430-1496 | 1473-1523 | 2414-2539 | 1569-1648 |
| 90 | 60 | 1 | 12 | 12-13 | 20-21 | 13-14 |
| | 70 | | 15-16 | 16 | 26-27 | 17-18 |
| | 80 | | 20-21 | 21 | 34-35 | 22-23 |
| | 60 | 0.5 | 46-48 | 48-49 | 78-82 | 51-53 |
| | 70 | | 60-63 | 62-64 | 101-106 | 66-69 |
| | 80 | | 79-83 | 81-84 | 133-140 | 87-91 |
| | 60 | 0.3 | 127-133 | 131-136 | 215-226 | 140-147 |
| | 70 | | 166-174 | 171-177 | 280-295 | 182-192 |
| | 80 | | 218-228 | 225-232 | 368-387 | 240-252 |
| | 60 | 0.1 | 1143-1196 | 1178-1217 | 1930-2029 | 1255-1318 |
| | 70 | | 1493-1562 | 1538-1590 | 2520-2650 | 1638-1721 |
| | 80 | | 1961-2052 | 2020-2088 | 3311-3482 | 2152-2260 |
| 95 | 60 | 1 | 16-17 | 17 | 27-28 | 18 |
| | 70 | | 20-21 | 21 | 34-35 | 22-23 |
| | 80 | | 25-27 | 26-27 | 43-45 | 28-29 |
| | 60 | 0.5 | 63-66 | 65-67 | 105-111 | 69-72 |
| | 70 | | 79-82 | 81-84 | 133-140 | 86-91 |
| | 80 | | 100-105 | 103-107 | 169-177 | 110-115 |
| | 60 | 0.3 | 173-181 | 178-184 | 292-307 | 190-199 |
| | 70 | | 218-228 | 225-232 | 368-387 | 239-251 |
| | 80 | | 277-290 | 285-295 | 467-492 | 304-319 |
| | 60 | 0.1 | 1554-1626 | 1601-1655 | 2623-2759 | 1706-1791 |
| | 70 | | 1958-2048 | 2017-2085 | 3305-3476 | 2149-2257 |
| | 80 | | 2489-2605 | 2565-2651 | 4203-4420 | 2732-2870 |

Given that the ultimate goal is to identify highly-active SlugCas9 variants with robust activities across all four PAMs, Precision@K (P@K) and Normalized Discounted Cumulative Gain (NDCG@K) were chosen as additional evaluation metrics to focus on the predictive power of models for top-performing variants. Specifically, P@10 and NDCG@10 evaluate the ability of models to accurately rank the top 10 variants, reflecting its capacity to identify the best candidates for further experimental validation. Similarly, Precision@50 and NDCG@50 assess the accuracy of the model's predictions for the top 50 variants, offering a broader perspective on its ability to identify a diverse pool of promising candidates. These metrics collectively provide a comprehensive evaluation framework, balancing the performance of models in predicting overall activity relationships and its ability to prioritize high-performing variants critical for downstream applications.

The results demonstrate that fine-tuning significantly enhances model performance across all seven evaluation metrics, particularly R2, Spearman, and Pearson (FIG. 8A). These improvements indicate that fine-tuning effectively optimizes the ability of models to capture the relationship between SlugCas9 variant sequences and their activities. When comparing fine-tuning strategies, models fine-tuned on the last layer of pLMs exhibited superior performance compared to those fine-tuned on the last two layers. This observation suggests that fine-tuning only the last layer allows the model to adapt to the task-specific data while preserving the underlying structural and contextual knowledge encoded in the pre-trained embeddings of ESM-2 and Saprot. In contrast, excessive fine-tuning of additional layers may disrupt these foundational properties, potentially diminishing the model's generalization capabilities. Scatter plots of the test datasets for the four PAMs, generated using ESM-2 fine-tuned on the last layer and Saprot fine-tuned on the last layer, reveal a strong correlation between the ground truth and the model predictions (FIGS. 8B and 8C). These results validate the reliability of the models in predicting SlugCas9 variant activities across diverse PAMs, providing a robust foundation for further application in SlugCas9 evolution.

Additionally, the performance of pLMs, ESM-2 and Saprot were compared with classical one-hot encoding-based ML models, including Random Forest (RF), Multi-Layer Perceptron (MLP), Linear Regression (LR), and Convolutional Neural Networks (CNNs). Across all seven evaluation metrics, pLMs consistently outperformed classical ML models and CNNs, demonstrating superior predictive accuracy and robustness (FIG. 8D). This performance gap highlights the advantages of pLMs in capturing the complex sequence-activity relationships inherent in the SlugCas9 evolution task. Unlike one-hot encoding models, which rely on manual feature extraction and may struggle to represent high-dimensional sequence data effectively, pLMs leverage contextual embeddings learned from large protein sequence databases. These embeddings provide richer, task-relevant features that enhance the ability of models to predict SlugCas9 variant activities accurately.

To further improve the accuracy and robustness of the predictions, a 5-fold ensemble strategy was employed, combining predictions from multiple models. This approach aggregates outputs from individual models to reduce variability and enhance overall performance. Analysis across the seven evaluation metrics revealed that the performance of the ensemble models was consistent, with minimal variation between individual folds (FIG. 8E). Among the models, the 5-fold ensemble of Saprot demonstrated slightly better performance compared to the 5-fold ensemble of ESM-2. The 5-fold ensemble strategy proved effective in mitigating model-specific biases and leveraging the strengths of each fold, resulting in robust predictions for SlugCas9 variants.

### Activity Inference and NGS Validation of SlugCas9 Variant

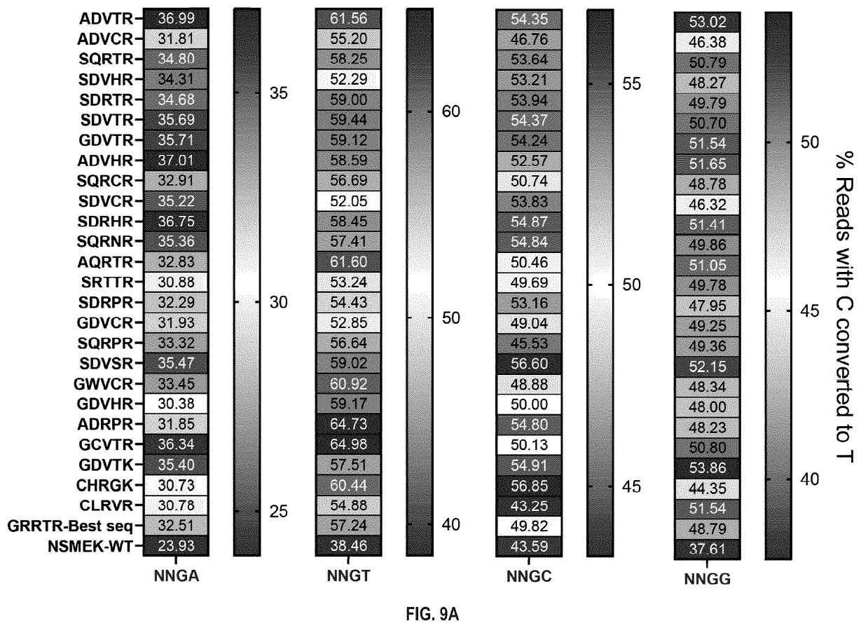

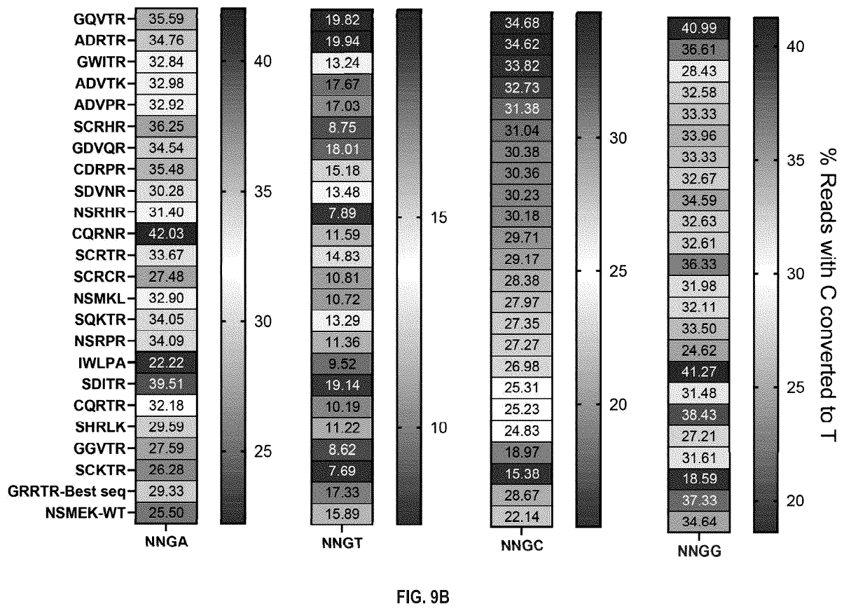

Activity inference was performed for all 5NNK combinations of SlugCas9 across four PAMs using 10 ensemble models (5-fold ESM-2 and 5-fold Saprot). Variants were ranked based on two criteria: average mutation numbers and average ranks across the predictions from the ensemble models. From the inference results, the top 25 variants ranked by average mutation numbers and the top 22 variants ranked by average ranks were selected for validation using NGS. This dual ranking approach ensures a comprehensive evaluation of predicted SlugCas9 variant activities, prioritizing both absolute mutation levels and relative performance consistency across models. NGS validation of the predicted SlugCas9 hits revealed numerous variants with activities surpassing SlugCas9-GRRTG, the top-performing variant identified from the large library sequencing data (FIGS. 9A and 9B). Notable examples include SlugCas9-ADVTR, SQRTR, SDVHR, ADVHR, GQVTR, and ADRTR, all of which exhibited robust activity across the four PAMs. Furthermore, the ranking strategy based on average mutation numbers proved more effective in identifying high-performing variants compared to the average ranking strategy. This suggests that prioritizing absolute activity levels provides a more reliable criterion for selecting optimal SlugCas9 variants for further development and applications.

Results demonstrate that the SDEML platform successfully identified high-performing SlugCas9 variants with enhanced activities across diverse PAMs. Compared to the top variant (SlugCas9-GRRTG) identified from the initial large library sequencing data, several new variants exhibited superior activity profiles, highlighting the effectiveness of SDEML in discovering optimal candidates. These findings validate the capability of SDEML to efficiently generate sequence-activity data and leverage advanced ML models to predict and prioritize variants with desirable functional properties, showcasing its potential as a powerful tool for protein evolution and engineering.

### Discussion

The SDEML platform outlined herein is a powerful tool that integrates high-throughput mutagenesis, NGS, and advanced ML models to establish sequence-activity landscapes for protein evolution. Unlike traditional evolution methods, which often discard information about low-performing variants, SDEML captures comprehensive sequence-activity data for all variants, enabling more informed predictions of protein activity. By leveraging pLMs and ensemble strategies, SDEML achieves high accuracy and resolution, significantly reducing the time and resources required for protein engineering. This approach not only provides an efficient way to screen protein libraries but also generates large-scale datasets for future applications in rational protein design.

SDEML enabled successful identification of several high-performing SlugCas9 variants with enhanced activity across diverse PAMs. Variants such as SlugCas9-ADVTR, SQRTR, and SDVHR demonstrated superior activity compared to SlugCas9-GRRTG, the best variant identified from the initial library. These results highlight the ability of SDEML to predicted SlugCas9 variants with broad PAM recognition The strong correlation between the predicted and validated activities underscores the reliability of the SDEML pipeline. These findings establish SDEML as an effective platform for discovering and optimizing protein variants, paving the way for its application in broader protein engineering challenges, including gene editing and therapeutic development.

### Expanded Applications of SDEML for the Evolution of Diverse Proteins

The SDEML platform can be further utilized for the evolution of various other proteins. The present disclosure further highlights the effectiveness and broad applicability of the SDEML platform through optimized plasmid design for diverse protein evolution using SDEML. For example, the SDEML platform can be used to evolve aminoacyl-tRNA synthetase (aaRS) (FIG. 11A). To achieve this, a TAG stop codon was introduced after the first methionine (Met) of the rAPOBEC1-dCas9-UGI complex in the writing plasmid, along with the corresponding sgRNA. The aaRS library and recording barcode were incorporated into the recording plasmid, which also contained the gene of tRNA. If an aaRS exhibits high activity, it can incorporate more non-canonical amino acids (ncAAs) at the TAG codon within the rAPOBEC1-dCas9-UGI complex, leading to an increased production of the Cas9 complex and, consequently, a higher frequency of mutations in the recording barcode. This establishes a direct linkage between the activity of aaRS toward different ncAAs and the average mutation rate in the barcode, enabling the evolution of aaRS using the SDEML platform. Additionally, the SDEML platform can be utilized for the evolution of RNA polymerases. Using T7 RNA polymerase as an example (FIG. 11B), a T7 promoter was introduced upstream of the rAPOBEC1-dCas9-UGI complex in the writing plasmid, along with the corresponding sgRNA. In the recording plasmid, the recording barcode was positioned downstream of the T7 RNA polymerase library. If a T7 RNA polymerase variant exhibits high activity, it will initiate transcription from more T7 promoters, leading to increased expression of the base editor-Cas9 complex and, consequently, a higher frequency of mutations in the recording barcode. This establishes a direct correlation between T7 RNA polymerase activity and the number of mutations in the barcode, enabling the evolution of RNA polymerase using the SDEML platform. Furthermore, the SDEML platform can be utilized for the evolution of transcription factors (TFs) (FIG. 11C). A TF binding site was introduced upstream of the rAPOBEC1-dCas9-UGI complex in the writing plasmid. The TF library and recording barcode were incorporated into the recording plasmid along with the sgRNA. If a TF variant exhibits high activity, it will bind tightly to the TF binding site, leading to increased transcription of the base editor-Cas9 complex and, consequently, a higher frequency of mutations in the recording barcode. Therefore, the activity of a TF variant can be evaluated based on the number of mutations in the barcode, enabling the evolution of transcription factors using the SDEML platform. Other Cas proteins also can be evolved by SDEML. Using the ancestor of Cas proteins, TnpB, as an example (FIG. 11D), a TnpB library was introduced into the recording plasmid along with the recording barcode. The sgRNA was incorporated into a targeting plasmid. If a TnpB variant exhibits higher activity, it will facilitate the anchoring of rAPOBEC1 and UGI to the barcode more efficiently, resulting in an increased number of mutations. Therefore, the activity of TnpB can be correlated with the number of mutations in the recording barcode, enabling its evolution using the SDEML platform. The SDEML platform can be expanded to facilitate the evolution of various proteins. Additionally, SDEML can be applied to the evolution of other proteins and nucleotides beyond those demonstrated here.

### Example 2: Sequence Display—Based Generation of a Sequence—Activity Dataset for Uracil DNA Glycosylase Inhibitor (UGI) Variants

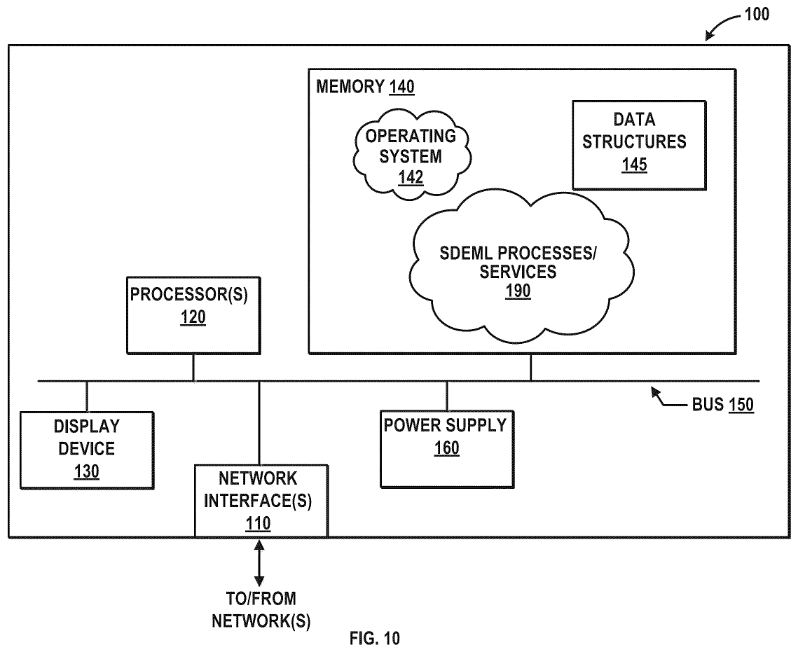

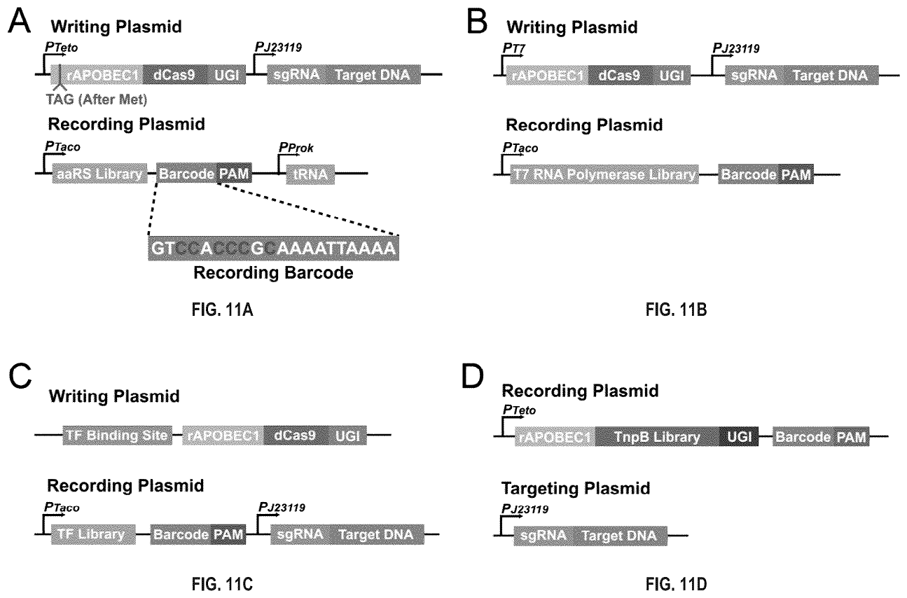

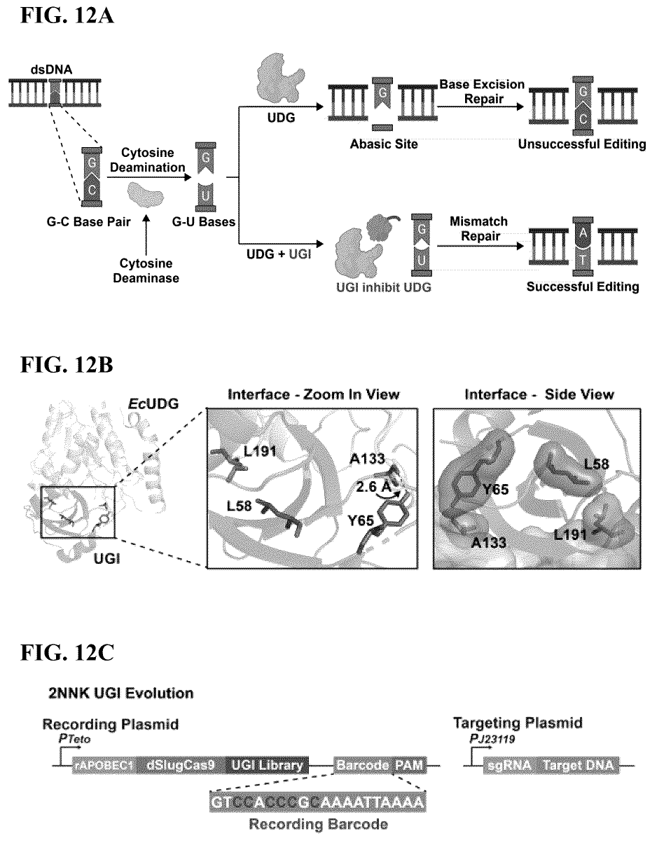

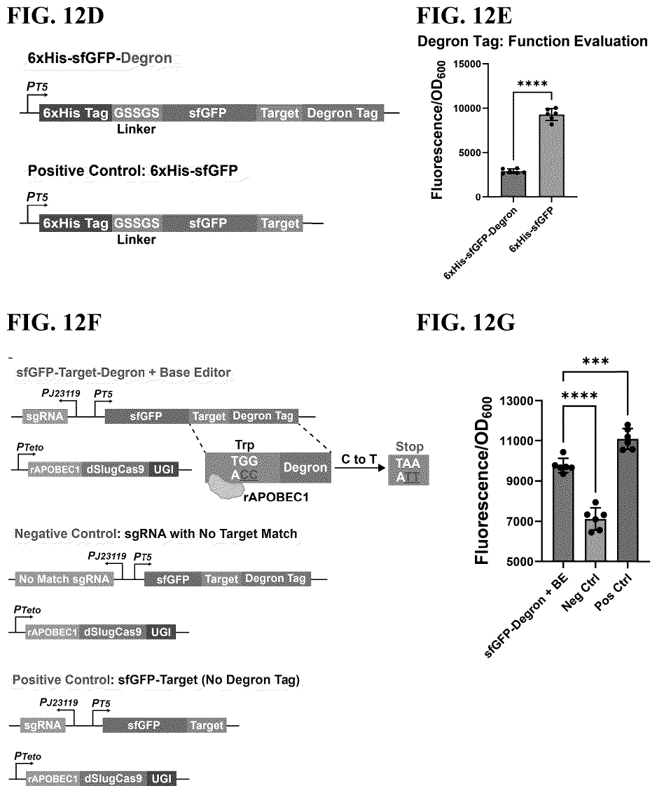

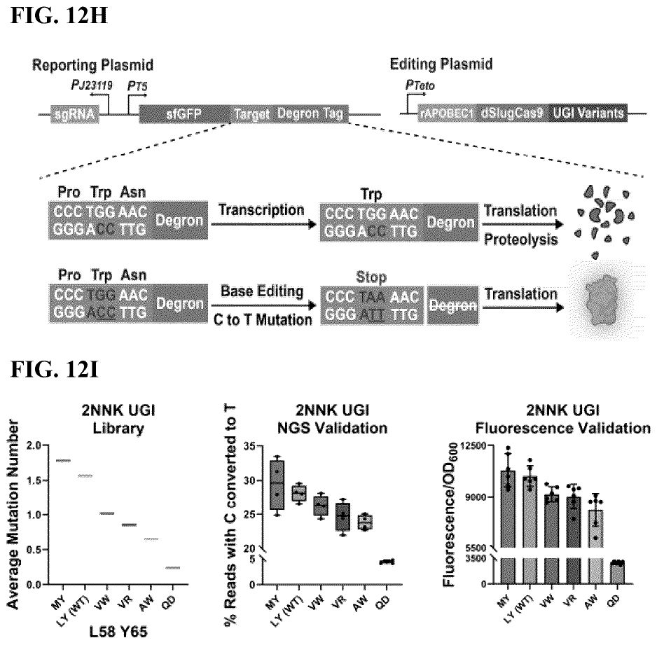

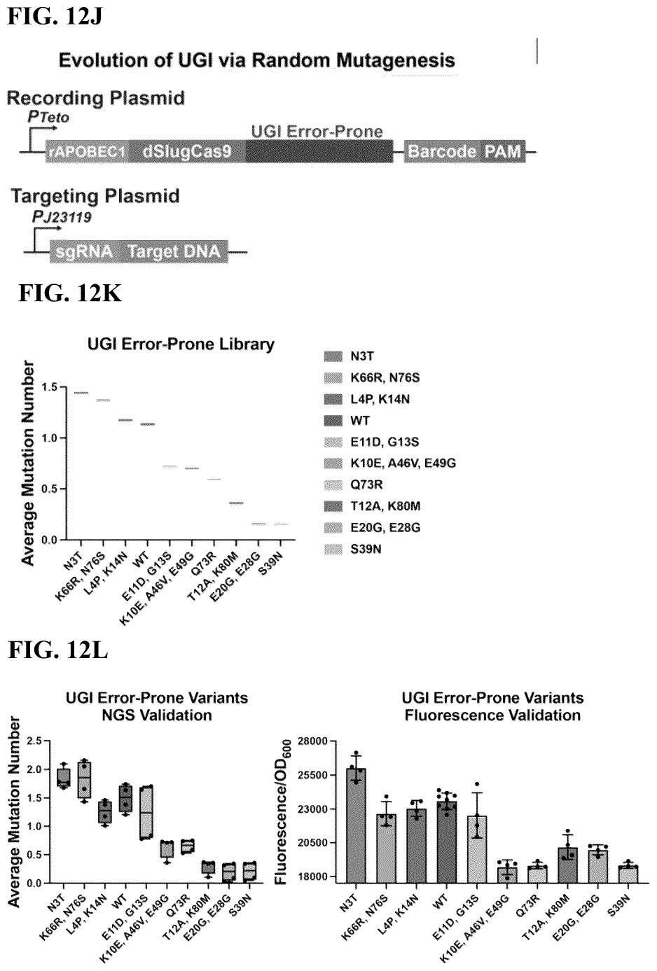

The following example describes, in some aspects, the effectiveness and accuracy of Sequence Display in obtaining sequence-activity datasets using UGI as a model protein. UGI is a protein inhibitor that specifically targets uracil-DNA glycosylase (UDG). When cytosine in double-stranded DNA (dsDNA) undergoes deamination, it is converted into uracil. UDG initiates base excision repair (BER) by removing the uracil base and forming an abasic site (a site lacking a base). UGI inhibits UDG activity by preventing its binding to uracil-containing DNA, thereby preserving uracil in its original position. This inhibition triggers the mismatch repair mechanism, which incorporates an adenine opposite the uracil, ultimately resulting in a C-to-T mutation (FIG. 12A).

A sequence-activity dataset was first created for a two-position UGI library. Based on the crystal structure of the UGI-EcUDG complex, a UGI library containing two randomized positions (Leu58 and Tyr65) involved in the recognition of EcUDG was generated (FIG. 12B). The co-crystal structure of the UGI-EcUDG complex revealed that Leu58 engaged in hydrophobic interactions with Leu191 of EcUDG, while Tyr65 formed a hydrogen bond with Ala133 of EcUDG, underscoring their crucial roles in binding to EcUDG. The Leu58 and Tyr65 residues were randomized to construct a 2NNK (N=any nucleotide, K=G or T) UGI library for Sequence Display.

This library was co-expressed with rAPOBEC1 and a catalytically dead SlugCas9 (dSlugCas9) in a recording plasmid, which contained a recording barcode and the PAM sequence downstream of the UGI library. In the targeting plasmid, an sgRNA complementary to the recording barcode was expressed, thereby directing the fusion base editing complex containing rAPOBEC1, dSlugCas9, and UGI to the barcode locus (FIG. 12C). The recording barcode was designed as a 20-nucleotide (nt) DNA sequence (GTCCACCCGCAAAATTAAAA) with six cytosine (C) sites capable of being converted to thymine (T) by the complex. A highly active UGI variant effectively inhibited UDG, leading to an increased frequency of C-to-T mutations in the recording barcode. In contrast, a low-activity variant resulted in fewer mutations.

The constructed 2NNK UGI library underwent a three-day Sequence Display reaction in liquid culture to encode the activity of each variant into barcodes, followed by NGS analysis. This process generated a sequence-activity dataset comprising 64,603 reads for 376 UGI variants, with 98 variants having more than 60 reads. Within this dataset, UGI-WT had an average mutation number of 1.566. Notably, some variants in the dataset exhibited higher average mutation numbers than UGI-WT, indicating enhanced activity. For example, UGI-MY (L58M, Y65) had an average mutation number of 1.783, and UGI-LM (L58, Y65M) had an average mutation number of 1.676, suggesting that these variants were more active than UGI-WT.

To validate the accuracy of this sequence-activity dataset, a degron-based fluorescence assay was established (FIGS. 12D-12G). A degron tag is a short peptide sequence (AANDENYALAA) that targets proteins for proteasomal degradation and is widely used to modulate protein expression in functional assays. The degron tag was fused to sfGFP via a Trp codon-containing linker that underwent rapid degradation, thereby resulting in low fluorescence in the absence of gene editing. With an efficient gene editing system, the base editor converted the Trp codon (TGG) into a stop codon (UAG, UGA, or UAA), removing the degron tag and restoring fluorescence. Consequently, this degron-based fluorescence assay served as a direct and quantitative readout of UGI variant activity.

This assay was then used to assess the accuracy of the sequence-activity dataset for UGI (FIG. 12H). Five variants were randomly selected from the dataset, and their activity was evaluated using the average mutation number, with comparison to UGI-WT. To confirm the accuracy of the dataset obtained from Sequence Display, NGS and fluorescence validation assays were also performed on these five variants alongside UGI-WT.

Based on the Sequence Display results, UGI-MY and UGI-WT exhibited the highest activity levels and were associated with the highest average mutation numbers. Variants UGI-VW, UGI-VR, and UGI-AW showed intermediate activity, corresponding to moderate average mutation numbers. In contrast, UGI-QD demonstrated the lowest activity with the lowest average mutation number (FIG. 12I). Consistent with the Sequence Display results, NGS and fluorescence validation assays further confirmed that UGI-MY and UGI-WT exhibited the highest C-to-T conversion percentages and fluorescence signals. Variants UGI-VW, UGI-VR, and UGI-AW demonstrated intermediate activity across both assays, following the trend VW>VR>AW, which was in line with their moderate C-to-T conversion rates and fluorescence intensities. UGI-QD consistently showed the lowest activity, with the lowest C-to-T conversion percentage and fluorescence signal, identifying it as the least functional variant (FIG. 12I). The validation results from both NGS and fluorescence assays aligned with the Sequence Display data, demonstrating a consistent activity trend across both the variants and UGI-WT. These results confirmed the high accuracy of the sequence-activity data generated by Sequence Display.

In addition to the focused UGI library, a random UGI protein library was prepared using error-prone PCR (FIG. 12J). For the experimental pipeline, this library was processed through the Sequence Display workflow. NGS simultaneously captured UGI variant sequences and their corresponding recording barcodes, thereby enabling generation of a comprehensive sequence-activity dataset.

To validate the accuracy of this dataset, nine variants along with UGI-WT were selected, and their activity was validated using NGS and a fluorescence assay (FIGS. 12K-12L). Both assays confirmed that variants with higher average mutation numbers in the barcodes exhibited greater activity.

To further quantitatively assess the data quality of the random mutagenesis library, the correlation between the average mutation number derived from the error-prone Sequence Display library and the fluorescence intensity measured by fluorescence assays was analyzed.

The results showed strong concordance between the two readouts, with a Pearson correlation coefficient of r=0.825, R²=0.681, and a Spearman rank correlation coefficient of p=0.733, indicating a strong correlation between activities measured by Sequence Display and the fluorescence assay.

Together, these results demonstrated that Sequence Display could also be accurately used to record the activities of a protein error-prone library.

### Example 3: Sequence Display-Based Generation of a Sequence-Activity Dataset for rAPOBEC1 Cytosine Deaminase Variants

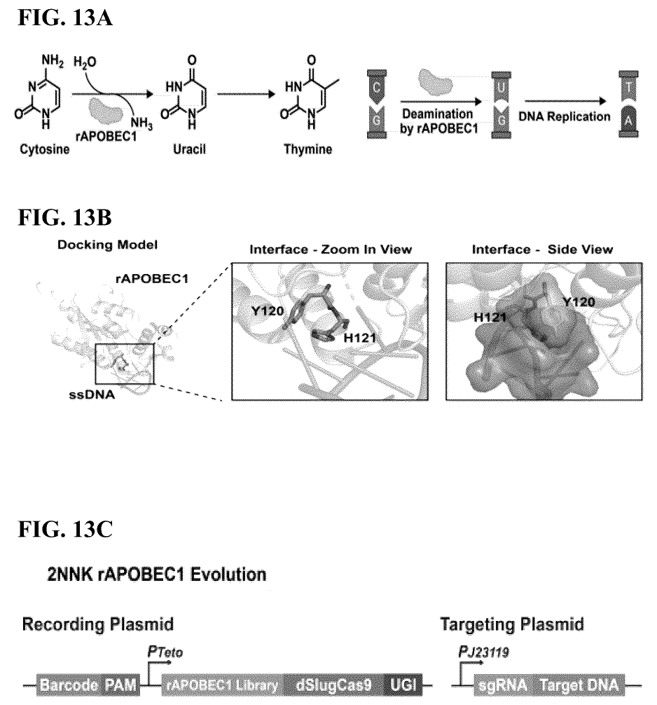

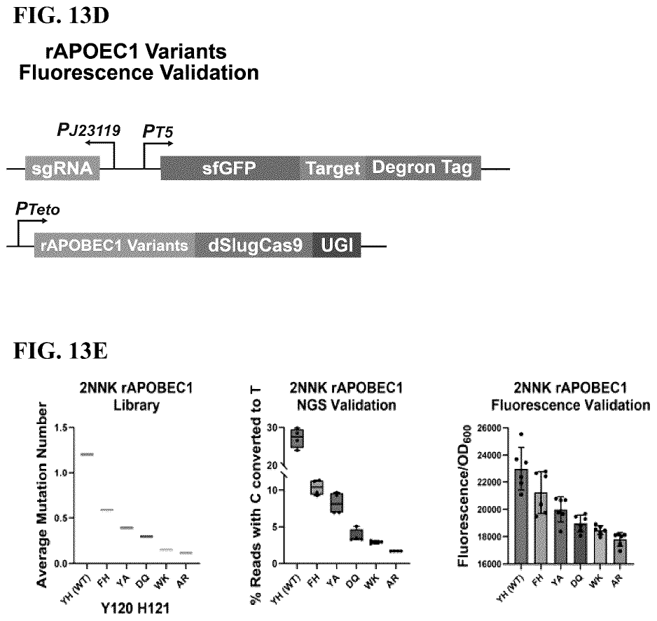

The following example described, in some aspects, the effectiveness and accuracy of Sequence Display in obtaining sequence-activity datasets using rAPOBEC1 as a model protein. The versatility of the Sequence Display methodology described herein was demonstrated by obtaining sequence-activity datasets for other proteins, such as rAPOBEC1. rAPOBEC1 is a cytosine deaminase that catalyzed the conversion of cytosine to uracil by removing the amino group from cytosine in single-stranded DNA (ssDNA). During DNA replication, this modification led to a C-to-T mutation (FIG. 13A).

To generate a sequence-activity dataset for rAPOBEC1, a two-position rAPOBEC1 library was constructed. Using AlphaFold2, the structure of rAPOBEC1 was predicted and docked with ssDNA (FIG. 13B). The predicted structure of the rAPOBEC1-ssDNA complex revealed two key residues, Tyr120 and His121, which had previously been shown to affect ssDNA binding and rAPOBEC1 catalytic activity. An rAPOBEC1 library of Tyr120 and His121 was inserted into a recording plasmid containing a recording barcode and PAM, while the target plasmid carried an sgRNA and a target DNA sequence (FIG. 13C).

A similar Sequence Display reaction as discussed hereinabove was then carried out, resulting in a sequence-activity dataset comprising 41,568 reads for 324 rAPOBEC1 variants, with 82 variants having more than 60 reads. Subsequently, a degron-based fluorescence assay for rAPOBEC1 was developed (FIG. 13D).

To validate the accuracy of this dataset, five variants along with rAPOBEC1-WT were selected, and their activity was validated using an NGS assay and a degron-based fluorescence assay. The NGS and degron-based fluorescence assays confirmed that variants with higher average mutation numbers in the barcodes exhibited greater activity (FIG. 13E).

Differences in detection thresholds, dynamic range, and resolution between fluorescence assays and Sequence Display introduced intrinsic assay-specific biases; therefore, quantitative discrepancies for individual variants, particularly near sensitivity limits, were expected and did not reflect reduced data quality. Accordingly, the robustness of Sequence Display datasets was evaluated based on consistency in relative activity ranking and preservation of global sequence-activity landscape features, rather than exact numerical agreement for individual variants.

### Example 4: Sequence Display-Based Evolution of Aminoacyl-tRNA Synthetase (aaRS) Variants Toward Non-Canonical Amino Acids

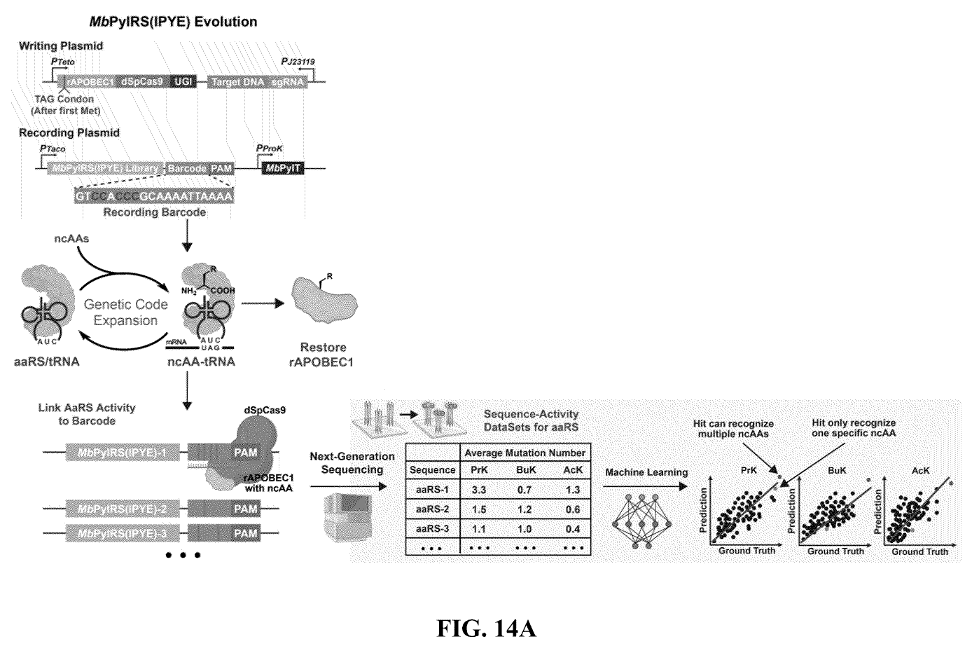

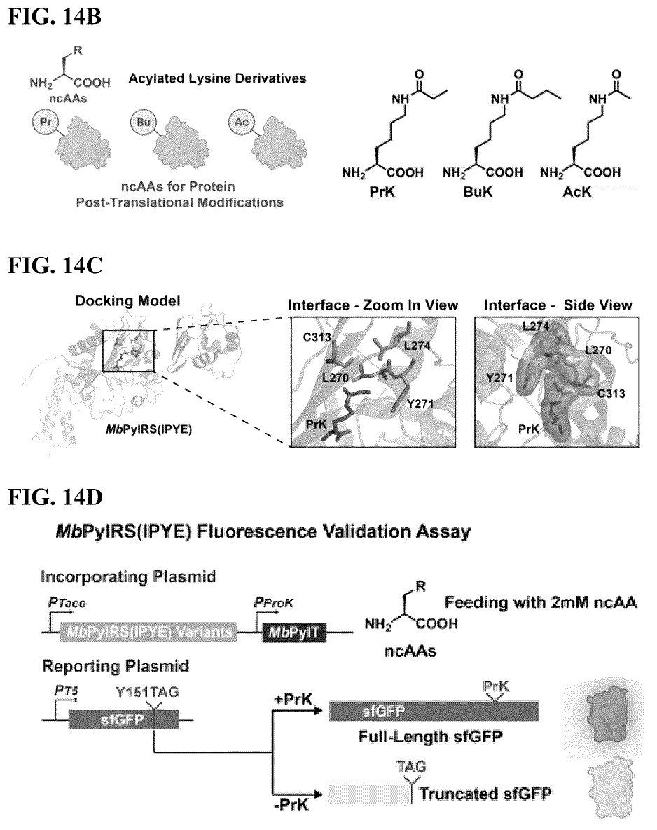

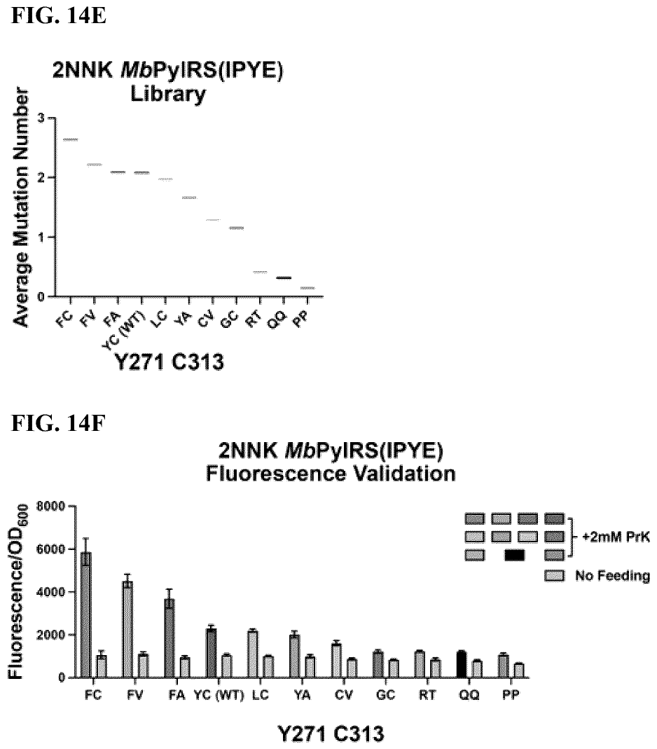

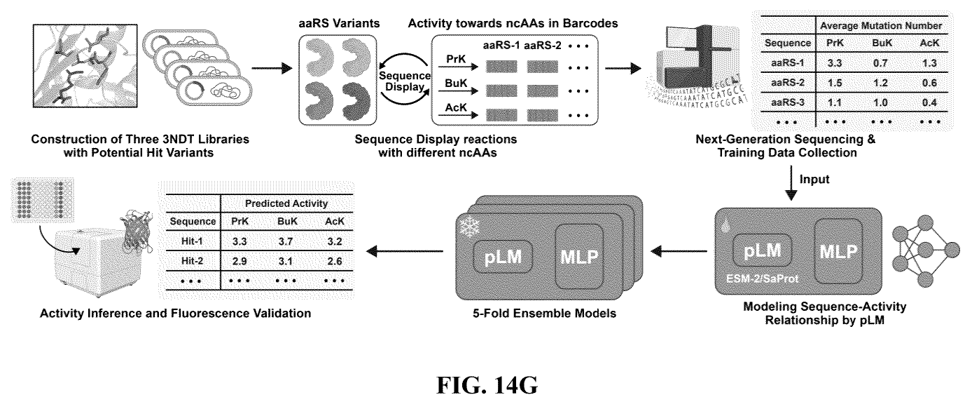

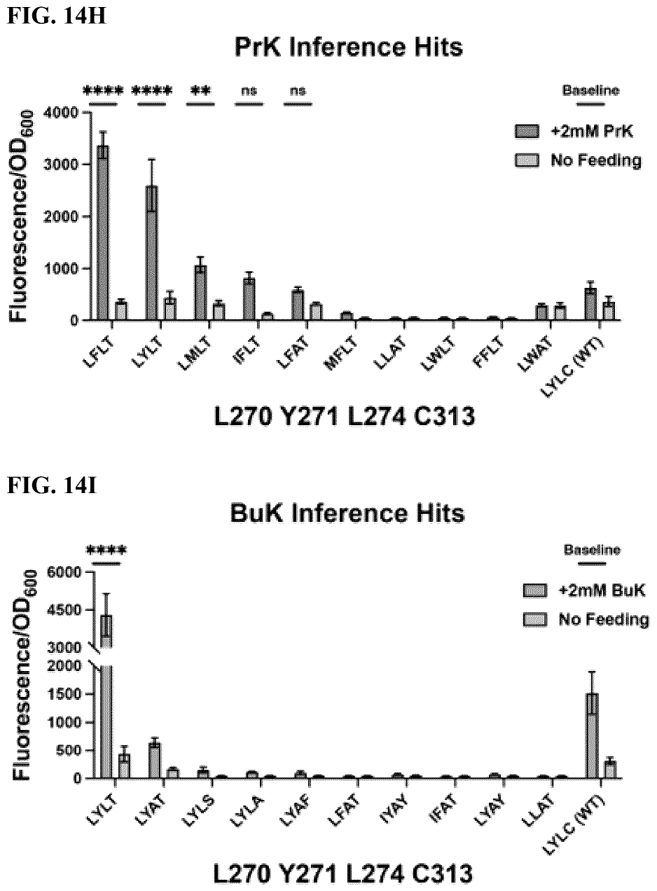

To demonstrate that Sequence Display was a versatile platform for protein evolution, the evolution of aaRS against ncAAs (i.e., acylated lysine derivatives serving as chemical mimics of lysine acylation PTMs) was carried out. aaRSs are essential enzymes that charge tRNAs with their cognate amino acids, thereby ensuring accurate translation of the genetic code. Genetic code expansion (GCE) technology was used to engineer the aaRS binding pocket, shifting its specificity toward ncAAs and enabling their site-specific incorporation into proteins (FIG. 14A). This technology introduced novel chemical functionalities, providing powerful tools for probing protein function and creating proteins with new or enhanced properties.

The aminoacyl-tRNA synthetase Methanosarcina barkeri PylRS(IPYE) (MbPylRS(IPYE)), which enables the incorporation of diverse ncAAs, was selected. Using Sequence Display, this synthetase was first evolved for improved activity toward the ncAA propionyllysine (PrK) (FIG. 14B) by constructing a two-position library. Using AlphaFold2, the structure of MbPylRS(IPYE) was predicted and docked with PrK (FIG. 14C). The predicted structure of the MbPylRS(IPYE)-PrK complex revealed four key residues-Leu270, Tyr271, Leu274, and Cys313-which had previously been shown to affect MbPylRS incorporation efficiency. An MbPylRS(IPYE) library with mutations at Tyr271 and Cys313 was cloned into a recording plasmid containing a recording barcode with a PAM site and MbPylT (FIG. 14A).

The writing plasmid encoded a fusion base editing complex containing rAPOBEC1, dSlugCas9, and UGI, together with an sgRNA complementary to the recording barcode. A TAG amber codon was introduced at the second position of rAPOBEC1. MbPylRS(IPYE) variants with high incorporation efficiency toward PrK restored rAPOBEC1 activity by incorporating PrK at this amber site, thereby reactivating the base editor system to introduce mutations into the recording barcodes. Consequently, the number of mutations in the barcodes served as a quantitative proxy for the PrK incorporation efficiency of MbPylRS(IPYE) variants (FIG. 14A).

A Sequence Display reaction was then performed in the presence of 2 mM PrK, yielding a sequence-activity dataset comprising 126,344 reads across 400 MbPylRS(IPYE) variants, with 234 variants represented by more than 60 reads. To validate the accuracy of this dataset, a fluorescence assay based on readthrough of the amber codon of GFP was used (FIG. 14D). Ten variants along with MbPylRS(IPYE)-WT were selected, and their activity was validated using this fluorescence assay.

Based on the Sequence Display results, MbPylRS(IPYE)-FC (Y271F, C313C) and MbPylRS(IPYE)-FV (Y271F, C313V) exhibited the highest activity, as indicated by the greatest average mutation numbers in the barcodes. MbPylRS(IPYE)-WT and MbPylRS(IPYE)-CV (Y271C, C313V) showed intermediate activity with moderate mutation numbers, while MbPylRS(IPYE)-QQ (Y271Q, C313Q) and MbPylRS(IPYE)-PP (Y271P, C313P) displayed the lowest activity, corresponding to the fewest mutations (FIG. 14E). These results were consistent with fluorescence assay measurements (FIG. 14F), indicating that Sequence Display efficiently captured the activity of large mutant libraries and identified variants with superior activity.

In addition, to demonstrate that Sequence Display-generated sequence-activity datasets could be used to evolve aaRS variants toward diverse ncAAs, Sequence Display was applied to generate MbPylRS(IPYE) datasets for multiple ncAA substrates, including propionyllysine (PrK), butyrylysine (BuK), and acetyllysine (AcK) (FIG. 14A-B). Guided by the docking model (FIG. 14C), three focused 3NDT (N=any nucleotide; D=A, G, or T) libraries were designed targeting four key residues: Library 1 targeted Leu270, Tyr271, and Cys313; Library 2 targeted Tyr271, Leu274, and Cys313; and Library 3 targeted Leu270, Tyr271, and Leu274. These libraries were pooled to generate the final screening library.

This library was then subjected to Sequence Display reactions in the presence of individual ncAAs. Following NGS, ncAA-specific sequence-activity datasets, together with the previously generated 2NNK dataset, were used to train protein language models (pLMs) (ESM-2 and SaProt) coupled with downstream multilayer perceptrons (MLPs). A five-fold ensemble strategy was subsequently applied. All combinations of the four targeted residues in MbPylRS(IPYE) were evaluated by activity inference, and the top-ranked variants were selected for fluorescence-based validation (FIG. 14G).

Using this approach, multiple MbPylRS(IPYE) variants with enhanced recognition of PrK, BuK, or AcK relative to MbPylRS(IPYE)-WT were identified. For example, MbPylRS(IPYE)-LFLT (Y271F, C313T), -LYLT (C313T), and -LMLT (Y271M, C313T) exhibited significantly increased incorporation activity toward PrK compared with the WT (FIG. 1411). MbPylRS(IPYE)-LYLT also showed a significant enhancement in incorporation activity toward BuK (FIG. 14I). In addition, MbPylRS(IPYE)-ILAF (L270I, Y271L, L274A, C313F) and -LFLT (Y271F, C313T) displayed significantly improved incorporation activity toward AcK relative to MbPylRS(IPYE)-WT (FIG. 14J).

Collectively, these results demonstrated that Sequence Display enabled identification of aaRS variants with enhanced activity and specificity toward distinct ncAAs.

### Example 5: Sequence Display-Based Generation of a Sequence-Activity Dataset for SlugCas9 Variants with Diverse PAM Recognition

SlugCas9 (1054 amino acids), guided by a single-guide RNA (sgRNA), precisely targeted specific DNA sequences and induced a double-stranded break at the designated site. In comparison, widely used Cas9 proteins, such as SpCas9 (1368 amino acids) and others (e.g., NmCas9 (1082 amino acids), GeoCas9 (1087 amino acids), St1Cas9 (1121 amino acids), and St3Cas9 (1388 amino acids)), were too large to be efficiently delivered via a single adeno-associated virus (AAV) for in vivo therapies. SlugCas9 was a compact Cas9 nuclease that could be readily packaged into a single AAV. However, the limited PAM recognition of compact Cas9 nucleases posed a challenge for their use in gene editing applications.

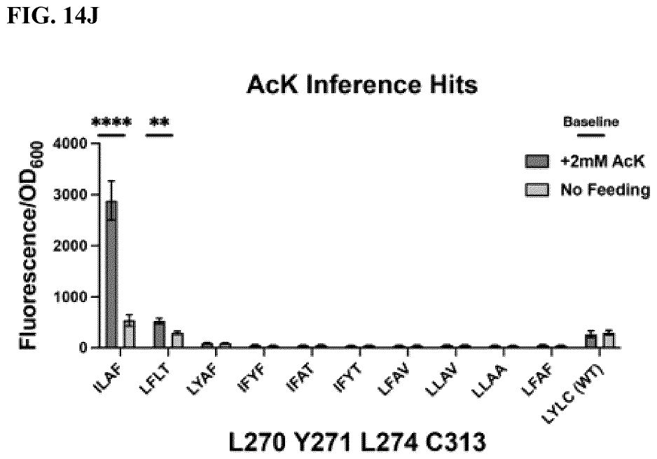

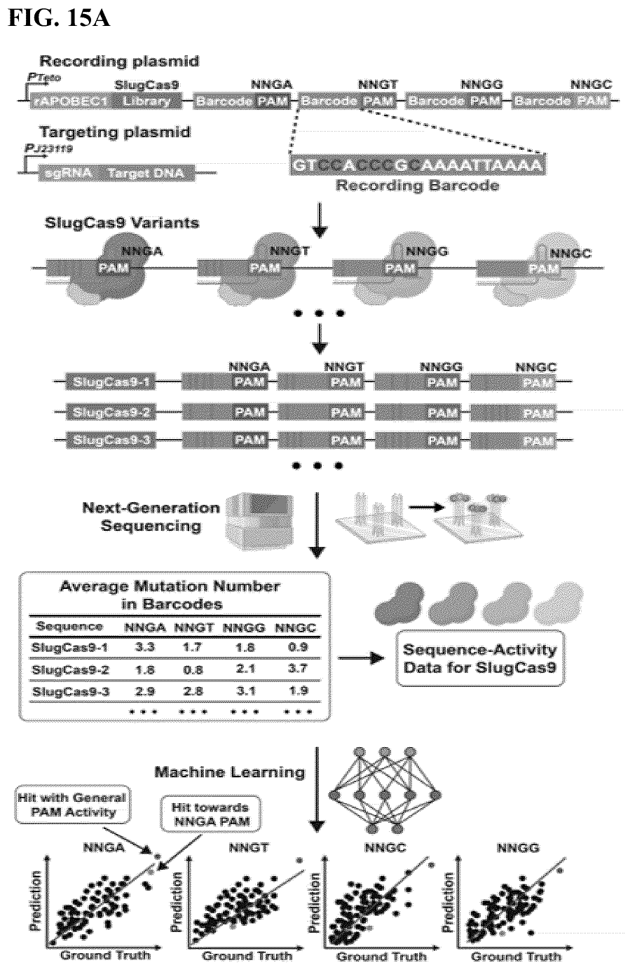

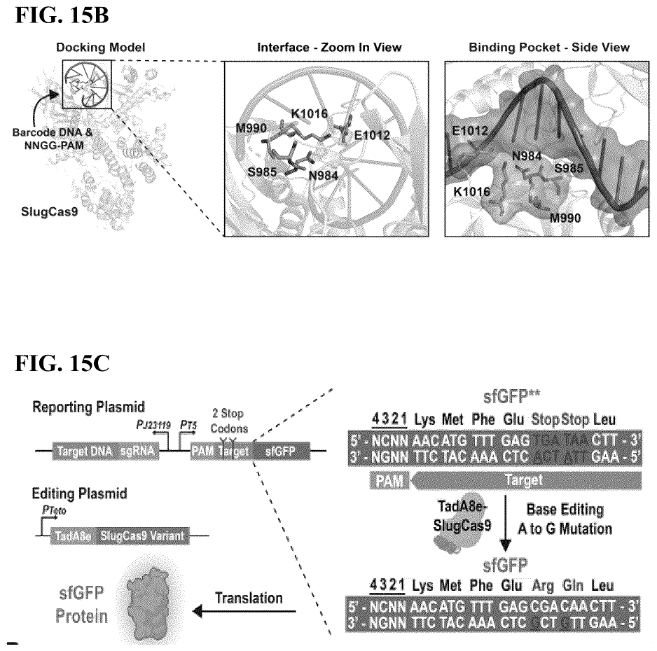

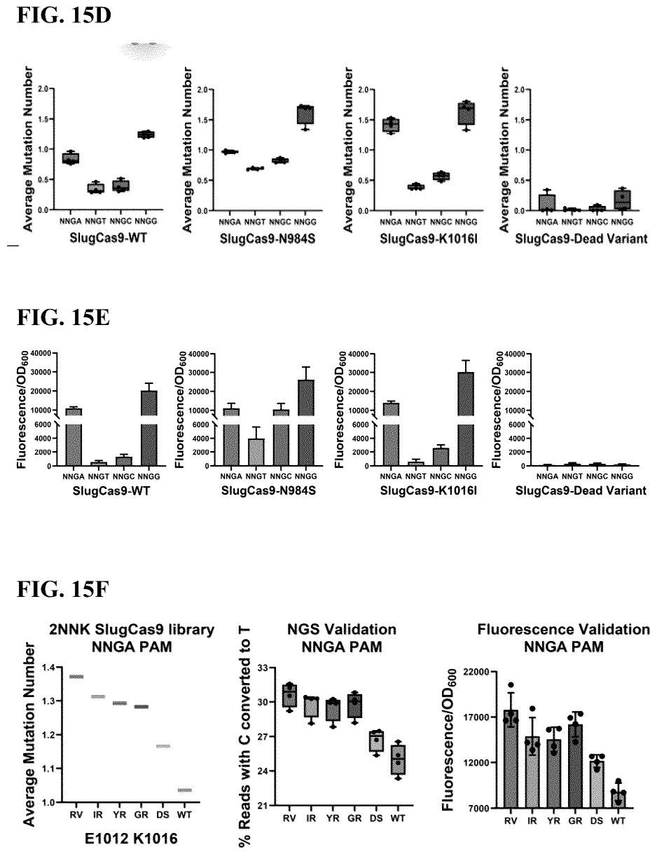

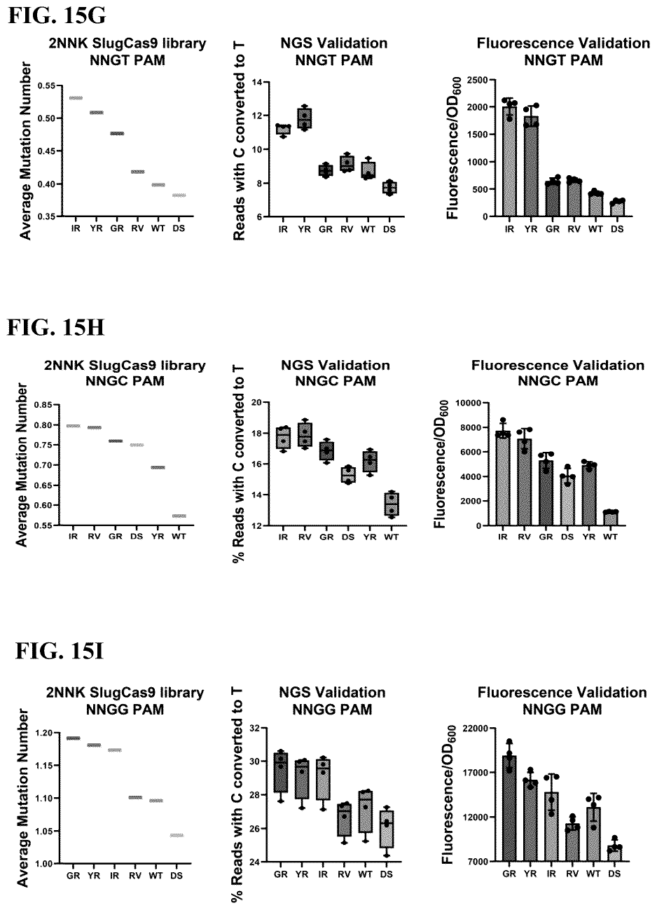

It was next demonstrated that Sequence Display could accurately capture the activity of SlugCas9 variants across different PAM sequences using multiple barcodes (FIG. 15A). rAPOBEC1 and a dSlugCas9 library were incorporated into a recording plasmid containing four recording barcodes with different PAM sequences (NNGA, NNGT, NNGC, NNGG) downstream of the SlugCas9 gene. The recording barcodes were 20 bp DNA sequences containing six cytosine bases, which could mutate to thymine bases upon the action of rAPOBEC1. A separate targeting plasmid expressed an SaCas9-compatible sgRNA, which was recognized by SlugCas9 and guided the base-editing complex to the recording barcode, thereby enabling efficient barcode mutation.

To validate the accuracy of the multiplexed-barcoded Sequence Display system, a sequence-activity dataset was first generated for a SlugCas9 library containing mutations at two positions. The 3D structure of SlugCas9-WT was obtained from the AlphaFold Protein Structure Database (accession: AOA133QCR3) and docked with the recording barcode and an NNGG PAM (FIG. 15B). Based on the docking model, residues N984, M990, S985, E1012, and K1016 were identified as proximal to the Cas9 binding interface and the PAM motif. In addition, previous studies had shown that mutations at E1012 and K1016 could enhance Cas9 binding efficiency and expand its PAM recognition profile 62. Accordingly, E1012 and K1016 were selected to construct a 2NNK SlugCas9 library.

A similar Sequence Display reaction as described above was then performed, generating a sequence-activity dataset comprising 32,167 reads corresponding to 394 SlugCas9 variants, of which 152 variants had more than 60 reads. This dataset enabled assessment of each SlugCas9 variant's activity toward each PAM based on the average mutation number in the barcodes adjacent to the corresponding PAM sequence.

Due to the high fluorescence background observed in degron-based fluorescence assays, a TadA8e-based fluorescence assay was developed to validate the activity of different SlugCas9 variants across four PAMs (FIG. 15C). For this assay, a reporting plasmid was designed encoding an sgRNA and sfGFP fused to a 20 bp target sequence and a PAM sequence. The editing plasmid contained TadA8e together with the SlugCas9 variants. TadA8e is an adenine deaminase that catalyzes the conversion of adenine (A) to guanine (G) in ssDNA.

In the reporting plasmid, sfGFP was linked via a 20 bp target sequence containing two stop codons (TGA and TAA) to an N-terminal PAM sequence. Initially, these stop codons prevented sfGFP translation, resulting in no fluorescence signal. Deamination of adenine to guanine by TadA8e converted these stop codons into Arg (CGA) and Glu (CAA) codons, thereby allowing sfGFP translation to proceed, restoring its expression, and generating a strong fluorescence signal. The fluorescence intensity of sfGFP thus served as a quantitative indicator of the activity of each SlugCas9 variant toward specific PAM sequences.

To enhance the accuracy of the fluorescence assay, the promoter in the editing plasmid driving TadA8e-SlugCas9 expression was optimized. Fluorescence analysis showed that the Tet promoter produced the lowest background fluorescence and yielded a stronger fluorescence signal than the other promoters tested.

To evaluate the performance of the TadA8e-based fluorescence assay, SlugCas9-WT, two previously reported variants, and one dead variant were tested using both the Sequence Display reaction (FIG. 15D) and the fluorescence assay (FIG. 15E). SlugCas9-WT exhibited average mutation numbers in the order of NNGG>NNGA>NNGC>NNGT, consistent with previously reported activity trends and the fluorescence assay results. Furthermore, the variants SlugCas9-N984S and SlugCas9-K1016I showed enhanced activity across all four PAMs compared to SlugCas9-WT, in agreement with both prior studies and the fluorescence assay data. As expected, the dead variant displayed minimal activity and low fluorescence signals across all PAMs. These results confirmed the accuracy and reliability of the TadA8e-based fluorescence assay for validating activity data generated by Sequence Display.

To validate the accuracy of the 2NNK SlugCas9 dataset generated by the multiplexed-barcoded Sequence Display system, five variants along with SlugCas9-WT were selected, and their activity was assessed using both the NGS validation assay and the TadA8e-based fluorescence assay (FIGS. 15F-I).

Using the NNGA PAM mode as an example, SlugCas9-RV (E1012R, K1016V) exhibited the highest average mutation number compared to the other variants. SlugCas9-IR, -YR, and -GR showed moderate average mutation numbers, whereas SlugCas9-DS and SlugCas9-WT exhibited the lowest values. These findings were consistent with the NGS and fluorescence validation results, in which SlugCas9-RV demonstrated the highest C-to-T conversion rate and fluorescence signal; SlugCas9-IR, —YR, and -GR exhibited moderate conversion rates and fluorescence signals; and SlugCas9-DS and SlugCas9-WT displayed the lowest activity (FIG. 3F).

Similar trends were observed across the other three PAMs, further confirming the consistency of the Sequence Display results with the NGS and fluorescence validation results (FIGS. 3G-I). These results demonstrated that the multiplexed-barcoded Sequence Display system accurately correlated SlugCas9 PAM-specific activity with the corresponding genotype sequences.

### Genome and Base Editing by SlugCas9 Variants at Endogenous Loci

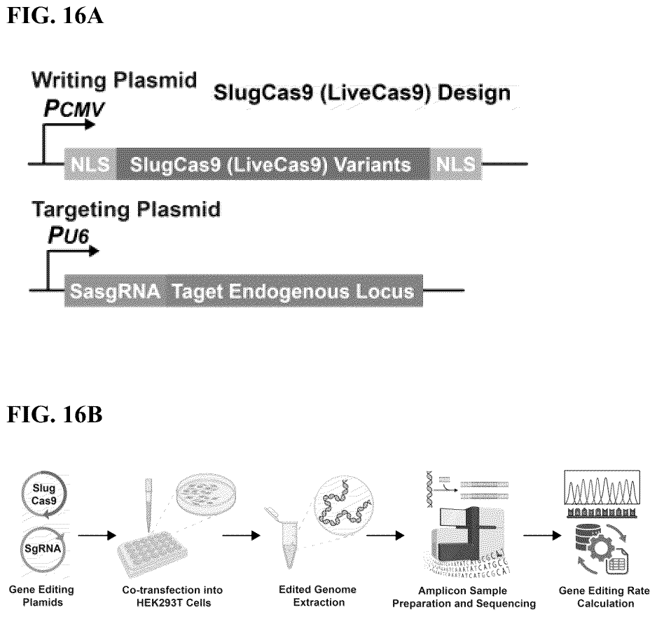

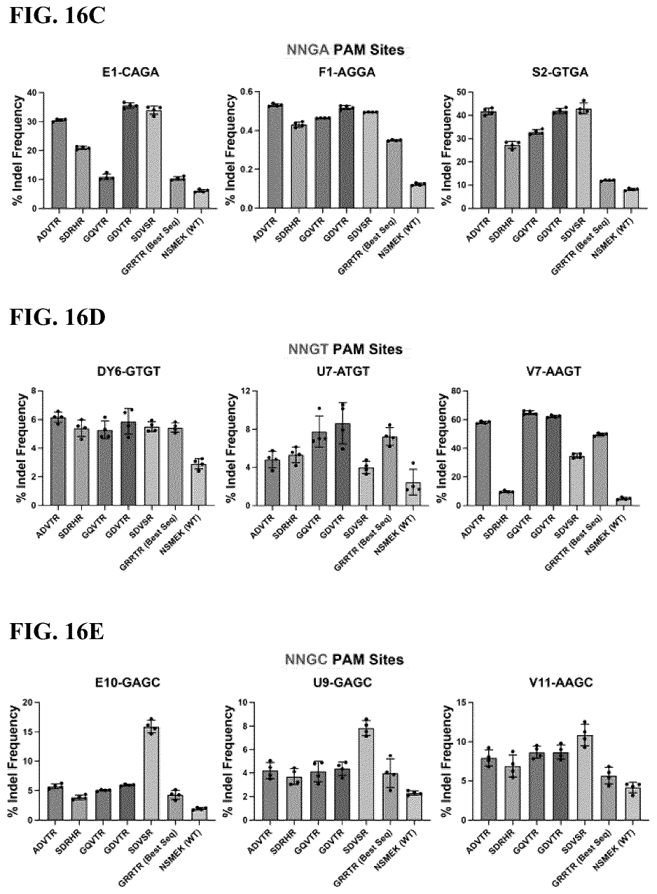

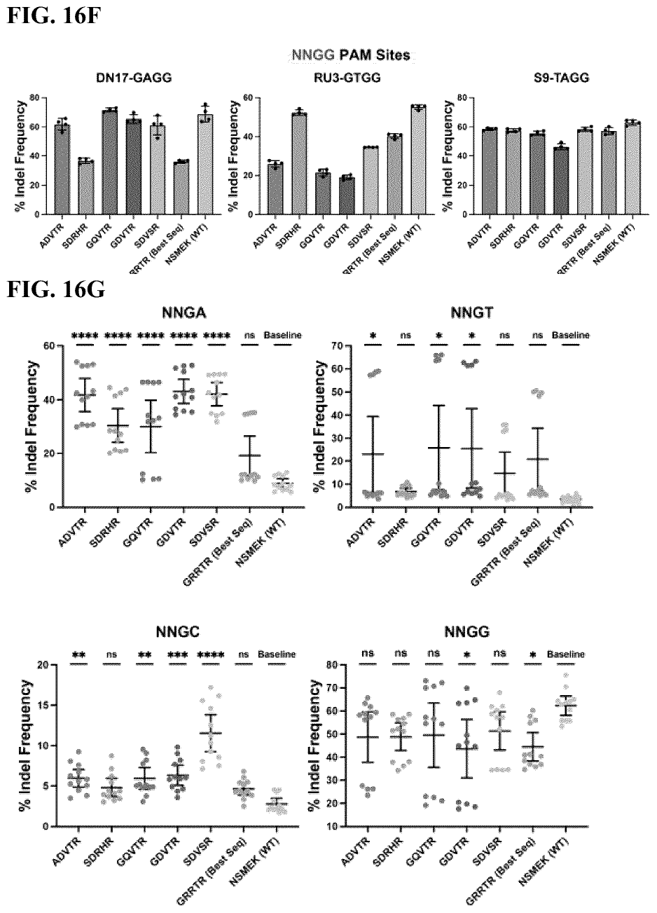

Next, the five top-performing SlugCas9 variants (SlugCas9-ADVTR, -SDRHR, -GQVTR, -GDVTR, and -SDVSR) identified by Sequence Display, together with the best sequencing-derived variant (SlugCas9-GRRTR), were evaluated in HEK293T cells, using SlugCas9-WT as a control. SlugCas9 (LiveCas9) expression plasmids were constructed (FIG. 16A), and 12 endogenous target sites bearing NNGN PAMs (three loci per PAM) were selected. Writing and targeting plasmids were co-transfected into HEK293T cells. After 3 days, genomic DNA was extracted and used to prepare amplicon libraries for NGS. Editing efficiencies were quantified from the sequencing data (FIG. 16B).

The five top-performing variants and the best sequencing-derived variant exhibited higher editing efficiencies than SlugCas9-WT at NNGA, NNGT, and NNGC PAMs (FIGS. 16C-E), while maintaining relatively strong activity at the native NNGG PAM compared with SlugCas9-WT (FIG. 16F). Although editing efficiencies at the native NNGG PAM were reduced relative to SlugCas9-WT, this trend was consistent with previously reported evolved SlugCas9 variants. This result likely reflected a trade-off in which mutations that broadened PAM recognition reconfigured the PAM-interacting interface, thereby weakening the optimal contacts required for efficient recognition of the native NNGG PAM.

Consistent with these findings, summary analyses of indel frequencies (FIG. 16G) and fold changes relative to SlugCas9-WT across the 12 endogenous target sites showed that these top variants exhibited significantly enhanced activity at NNGA, NNGT, and NNGC PAMs' PGP24T1 while retaining comparable activity at the native NNGG PAM (Table 3).

TABLE 3
Fold Change in Indel Frequency Relative to SlugCas9-WT

| | Fold Change | | | |
|---|---|---|---|---|
| SlugCas9 | NNGA | NNGT | NNGC | NNGG |
| ADVTR | 4.73x | 6.74x | 2.14x | 0.78x |
| SDRHR | 3.45x | 1.99x | 1.73x | 0.78x |
| GQVTR | 3.40x | 7.60x | 2.13x | 0.80x |
| GDVTR | 4.88x | 7.50x | 2.28x | 0.70x |
| SDVSR | 4.76x | 4.30x | 4.14x | 0.82x |
| GRRTR | 2.17x | 6.09x | 1.67x | 0.71x |

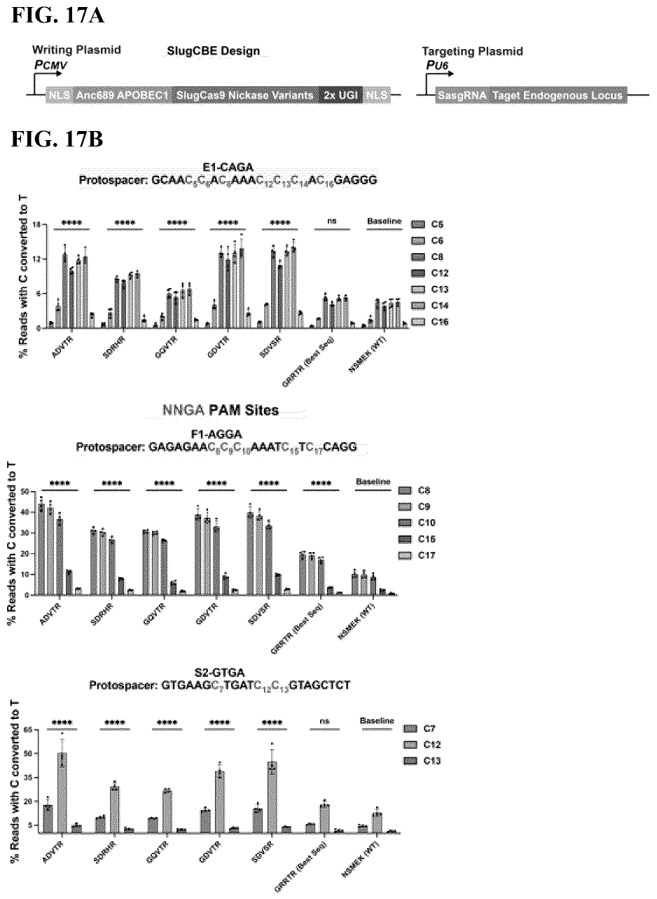

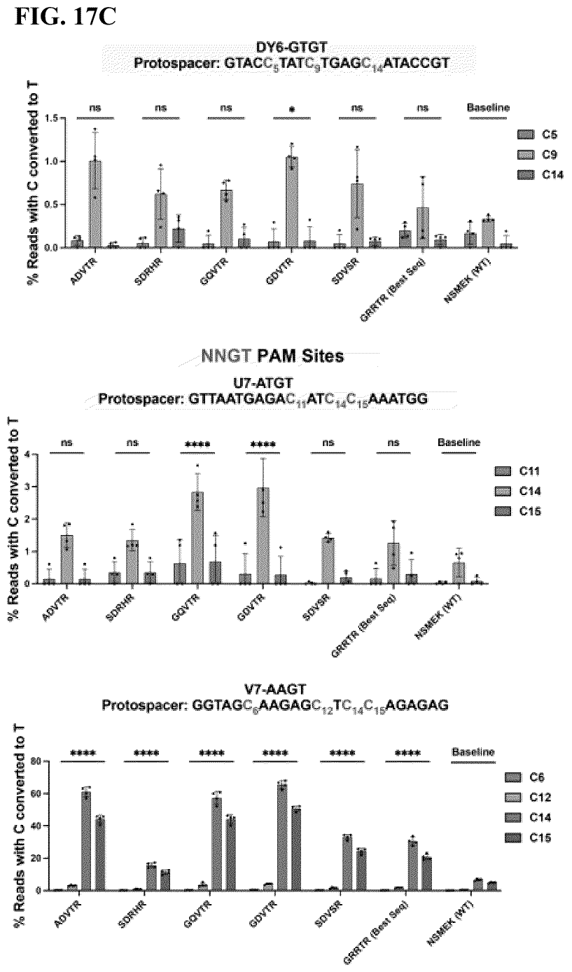

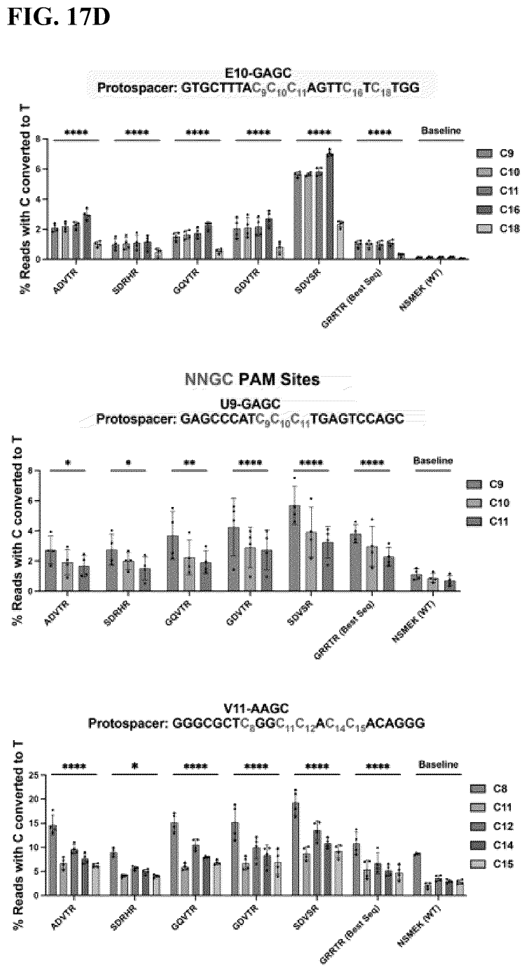

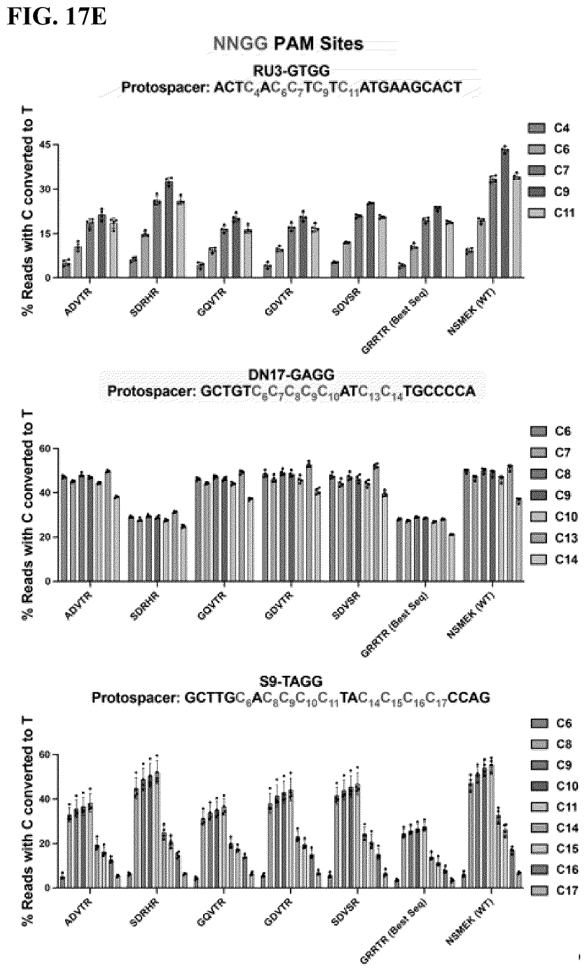

The base-editing capabilities of the five top-performing SlugCas9 variants and the best sequencing-derived variant were also evaluated in comparison with SlugCas9-WT. Anc689 APOBEC1 and two copies of UGI were fused to the nickase form of SlugCas9 to generate a cytidine base editor, termed SlugCBE (FIG. 17A).

Three days after transfection, the five top-performing SlugCBE variants exhibited higher C-to-T conversion efficiencies at the majority of NNGA, NNGT, and NNGC target sites than both the best sequencing-derived variant and SlugCBE-WT (FIGS. 17B-17D). These SlugCBE variants also retained editing efficiencies at the native NNGG PAM comparable to SlugCBE-WT (FIG. 17E).

Together, these results demonstrated that SlugCas9 variants identified through Sequence Display enabled efficient editing of endogenous target sites bearing diverse NNGN PAMs in mammalian cells. These variants substantially expanded the targeting scope of SlugCas9 beyond the native NNGG PAM while maintaining robust activity, thereby underscoring the capability of Sequence Display to rapidly evolve Cas9 proteins with enhanced and programmable PAM compatibility for genome and base-editing applications.

### Embodiments

Embodiment 1. A method, comprising: attaching a recording barcode to a gene encoding a protein of interest (POI); linking a function of the POI to an activity of a writer enzyme, thereby inducing sequence modifications in the recording barcode; and obtaining, at a processor in communication with a memory and through a sequencing method, sequence-function data including a sequence of the POI and the sequence modifications in the recording barcode.

Embodiment 2. The method of embodiment 1, further comprising: introducing, through a barcode hyper-mutagenesis reaction, the recording barcode into a recording plasmid adjacent to a protein amino acid sequence of the POI, the recording barcode corresponding with a protospacer adjacent motif (PAM) sequence, and the barcode hyper-mutagenesis reaction resulting in a POI variant having one or more mutations present in the recording barcode.

Embodiment 3. The method of embodiment 2, the sequence-function data including a barcode-mutated library of the POI variant, which encodes information about correlations between mutations present in the recording barcode and activity of the POI variant with respect to the PAM sequence.

Embodiment 4. The method of embodiment 3, further comprising: generating one or more large sequence-activity datasets including one or more barcode-mutated libraries for one or more POI variants with respect to one or more PAM sequences; and applying, at the processor in communication with the memory, a transfer learning process to a protein language model (pLM) based on the one or more large sequence-activity datasets.

Embodiment 5. The method of embodiment 2, further comprising: mapping, at the processor and using a protein language model (pLM) and based on the mutations present in the recording barcode, a barcode-mutated protein amino acid sequence associated with the POI variant to an activity of the POI variant with respect to the PAM sequence.

Embodiment 6. The method of embodiment 3, further comprising: predicting, at the processor and using a protein structure prediction model, a 3-dimensional structure of the POI variant based on a barcode-mutated library of the POI variant.

Embodiment 7. The method of embodiment 1, the POI being a Staphylococcus lugdunensis Cas9 nuclease.

### CLAUSES

Clause 1. A system comprising: (a) a plasmid library comprising a plurality of plasmids, each plasmid comprising: (i) a nucleic acid sequence encoding a variant of a protein of interest (POI); and (ii) a recording barcode comprising a nucleic acid sequence positioned in cis with the nucleic acid sequence encoding the variant of the POI; (b) a writer enzyme comprising a base-editing enzyme capable of introducing one or more sequence modifications in the recording barcode, wherein activity of the variant of the POI modulates expression or activity of the base-editing enzyme such that sequence modifications are introduced into the recording barcode in an activity-dependent manner; and (c) a sequencing and data-processing subsystem comprising: (i) a sequencing module configured to determine sequences of the nucleic acid sequence encoding the variant of the POI and the corresponding recording barcode; and (ii) at least one processor in communication with a memory storing instructions that, when executed, cause the processor to correlate sequence information of the variant of the POI with sequence modifications in the corresponding recording barcode to generate a sequence-activity dataset.

Clause 2. The system of clause 1, wherein the recording barcode comprises a synthetic nucleic acid sequence of about 10 to about 100 nucleotides.

Clause 3. The system of clause 1 or 2, wherein the recording barcode comprises a plurality of editable nucleotides susceptible to base substitution by the writer enzyme.

Clause 4. The system of any one of clauses 1 to 3, wherein the editable nucleotides comprise cytosine residues configured for C-to-T conversion.

Clause 5. The system of any one of clauses 1 to 3, wherein the editable nucleotides comprise adenine residues configured for A-to-G conversion.

Clause 6. The system of any one of clauses 1 to 5, wherein the recording barcode is positioned adjacent to the nucleic acid encoding the variant of the POI.

Clause 7. The system of any one of clauses 1 to 6, wherein each plasmid comprises a unique recording barcode sequence corresponding to a single POI variant.

Clause 8. The system of any one of clauses 1 to 7, wherein the writer enzyme comprises a base editor.

Clause 9. The system of clause 8, wherein the base editor comprises a deaminase fused to a programmable DNA-binding protein.

Clause 10. The system of clause 9, wherein the programmable DNA-binding protein comprises a Cas protein or Cas nickase.

Clause 11. The system of clause 9 or 10, wherein the programmable DNA-binding protein is guided to the recording barcode by a guide RNA.

Clause 12. The system of any one of clauses 1 to 11, wherein activity of the variant of the POI regulates transcription of the writer enzyme.

Clause 13. The system of any one of clauses 1 to 11, wherein activity of the variant of the POI regulates assembly or activation of the writer enzyme.

Clause 14. The system of any one of clauses 1 to 13, wherein the writer enzyme is encoded on the same plasmid as the POI variant.

Clause 15. The system of any one of clauses 1 to 13, wherein the writer enzyme is not encoded on the same plasmid as the POI variant.

Clause 16. The system of any one of clauses 1 to 15, wherein the POI is selected from the group consisting of a protease, a polymerase, a transcription factor, an aminoacyl-tRNA synthetase, a CRISPR-associated nuclease, an antibody or binding protein, and a protein-protein interaction partner.

Clause 17. The system of any one of clauses 1 to 16, wherein the POI comprises a protease and activity-dependent cleavage modulates activation of the writer enzyme.

Clause 18. The system of any one of clauses 1 to 16, wherein the POI comprises a polymerase and transcription from a polymerase-dependent promoter drives expression of the writer enzyme.

Clause 19. The system of any one of clauses 1 to 16, wherein the POI comprises a transcription factor and regulates a promoter controlling expression of the writer enzyme.

Clause 20. The system of any one of clauses 1 to 16, wherein the POI comprises an aminoacyl-tRNA synthetase and activity-dependent incorporation of a non-canonical amino acid restores activity of the writer enzyme.

Clause 21. The system of any one of clauses 1 to 16, wherein the POI comprises a CRISPR-associated nuclease and recognition of a target sequence modulates barcode editing.

Clause 22. The system of any one of clauses 1 to 16, wherein the POI comprises an antibody or binding protein and ligand binding modulates activation of the writer enzyme.

Clause 23. The system of any one of clauses 1 to 16, wherein the POI comprises a protein-protein interaction partner fused to a fragment of a split writer enzyme.

Clause 24. The system of any one of clauses 1 to 23, wherein the sequencing module comprises a next-generation sequencing platform.

Clause 25. The system of any one of clauses 1 to 24, wherein the sequencing module is configured to determine both the POI coding sequence and the corresponding recording barcode sequence in a single sequencing read.

Clause 26. The system of any one of clauses 1 to 25, wherein the processor calculates an edit frequency, an edit pattern, or a total number of edits within the recording barcode.

Clause 27. The system of any one of clauses 1 to 26, wherein the processor generates a quantitative activity score for each POI variant based on barcode modification metrics.

Clause 28. The system of any one of clauses 1 to 27, wherein the sequence-activity dataset comprises a plurality of POI variant sequences and corresponding activity values.

Clause 29. The system of clause 28, wherein the sequence-activity dataset is configured for use in training a machine learning model.

Clause 30. The system of clause 29, wherein the machine learning model comprises a protein language model.

Clause 31. The system of any one of clauses 1 to 30, wherein the plasmid library comprises variants generated by site-saturation mutagenesis or error-prone PCR.

Clause 32. The system of any one of clauses 1 to 31, wherein the plasmid library comprises variants targeting residues within a functional domain of the POI.

Clause 33. The system of any one of clauses 1 to 32, wherein the plasmid library is expressed in a prokaryotic cell.

Clause 34. The system of any one of clauses 1 to 32, wherein the plasmid library is expressed in a eukaryotic cell.

Clause 35. The system of any one of clauses 1 to 34, wherein the system records activity over multiple rounds of cell growth.

Clause 36. A method of generating a sequence-activity dataset for variants of a protein of interest (POI), the method comprising: (a) constructing a plasmid library comprising a plurality of plasmids, each plasmid comprising: (i) a nucleic acid sequence encoding a variant of the POI; and (ii) a recording barcode comprising a nucleic acid sequence positioned in cis with the nucleic acid sequence encoding the variant of the POI; (b) expressing the plasmid library in a cell in the presence of a writer enzyme capable of introducing one or more sequence modifications in the recording barcode; (c) coupling biological activity of each variant of the POI to expression or activity of the writer enzyme such that sequence modifications are introduced into the corresponding recording barcode in an activity-dependent manner; (d) sequencing the nucleic acid sequence encoding the variant of the POI and the corresponding recording barcode; and (e) using at least one processor in communication with a memory to correlate the nucleic acid sequence encoding the variant of the POI with sequence modifications in the corresponding recording barcode to generate a sequence-activity dataset.

Clause 37. The method of clause 36, wherein the recording barcode comprises a synthetic nucleic acid sequence of about 10 to about 100 nucleotides.

Clause 38. The method of clause 36 or 37, wherein the recording barcode comprises a plurality of editable nucleotides susceptible to base substitution by the writer enzyme.

Clause 39. The method of any one of clauses 36 to 38, wherein the editable nucleotides comprise cytosine residues configured for C-to-T conversion.

Clause 40. The method of any one of clauses 36 to 38, wherein the editable nucleotides comprise adenine residues configured for A-to-G conversion.

Clause 41. The method of any one of clauses 36 to 40, wherein each plasmid comprises a unique recording barcode corresponding to a single variant of the POI.

Clause 42. The method of any one of clauses 36 to 41, wherein the recording barcode is positioned adjacent to the nucleic acid encoding the variant of the POI.

Clause 43. The method of any one of clauses 36 to 42, wherein the writer enzyme comprises a base editor.

Clause 44. The method of clause 43, wherein the base editor comprises a nucleic acid deaminase fused to a programmable DNA-binding protein.

Clause 45. The method of clause 44, wherein the programmable DNA-binding protein comprises a Cas protein or Cas nickase.

Clause 46. The method of clause 44 or 45, wherein the programmable DNA-binding protein is directed to the recording barcode by a guide RNA.

Clause 47. The method of any one of clauses 36 to 46, wherein the writer enzyme is encoded on the same plasmid as the variant of the POI.

Clause 48. The method of any one of clauses 36 to 46, wherein the writer enzyme is not encoded on the same plasmid as the variant of the POI.

Clause 49. The method of any one of clauses 36 to 48, wherein biological activity of the variant of the POI regulates transcription of the writer enzyme.

Clause 50. The method of any one of clauses 36 to 48, wherein biological activity of the variant of the POI regulates assembly or activation of the writer enzyme.

Clause 51. The method of any one of clauses 36 to 50, wherein the POI is selected from the group consisting of a protease, a polymerase, a transcription factor, an aminoacyl-tRNA synthetase, a CRISPR-associated nuclease, an antibody or binding protein, and a protein-protein interaction partner.

Clause 52. The method of any one of clauses 36 to 51, wherein the POI comprises a protease and cleavage of a regulatory linker modulates activity of the writer enzyme.

Clause 53. The method of any one of clauses 36 to 51, wherein the POI comprises a polymerase and transcription from a polymerase-dependent promoter drives expression of the writer enzyme.

Clause 54. The method of any one of clauses 36 to 51, wherein the POI comprises a transcription factor that regulates a promoter controlling expression of the writer enzyme.

Clause 55. The method of any one of clauses 36 to 51, wherein the POI comprises an aminoacyl-tRNA synthetase and incorporation of a non-canonical amino acid restores activity of the writer enzyme.

Clause 56. The method of any one of clauses 36 to 51, wherein the POI comprises a CRISPR-associated nuclease and recognition of a target sequence modulates barcode editing.

Clause 57. The method of any one of clauses 36 to 51, wherein the POI comprises an antibody or binding protein and ligand binding modulates activation of the writer enzyme.

Clause 58. The method of any one of clauses 36 to 57, wherein the plasmid library comprises variants generated by site-saturation mutagenesis.

Clause 59. The method of any one of clauses 36 to 57, wherein the plasmid library comprises variants generated by error-prone PCR.

Clause 60. The method of any one of clauses 36 to 58, wherein the plasmid library comprises variants targeting residues within a functional domain of the POI.

Clause 61. The method of any one of clauses 36 to 60, wherein the plasmid library comprises at least 10², at least 103, or at least 104 distinct variants.

Clause 62. The method of any one of clauses 36 to 61, wherein the plasmid library is expressed in a prokaryotic cell.

Clause 63. The method of any one of clauses 36 to 61, wherein the plasmid library is expressed in a eukaryotic cell.

Clause 64. The method of any one of clauses 36 to 63, wherein the writer enzyme is induced for a period sufficient to permit accumulation of multiple sequence modifications in the recording barcode.

Clause 65. The method of any one of clauses 36 to 64, wherein the recording step comprises multiple rounds of cell growth and writer enzyme induction.

Clause 66. The method of any one of clauses 36 to 65, wherein sequencing comprises next-generation sequencing.

Clause 67. The method of any one of clauses 36 to 66, wherein sequencing comprises long-read sequencing.

Clause 68. The method of any one of clauses 36 to 67, wherein the nucleic acid encoding the variant of the POI and the corresponding recording barcode are sequenced within a single read.

Clause 69. The method of any one of clauses 36 to 68, further comprising amplifying the nucleic acid encoding the variant of the POI and the recording barcode prior to sequencing.

Clause 70. The method of any one of clauses 36 to 69, wherein correlating comprises calculating an edit frequency, edit pattern, or total number of edits within the recording barcode.

Clause 71. The method of any one of clauses 36 to 70, wherein the edit frequency or total number of edits is used to calculate a quantitative activity score for the variant of the POI.

Clause 72. The method of any one of clauses 36 to 71, wherein the sequence-activity dataset comprises a plurality of variant sequences and corresponding quantitative activity values.

Clause 73. The method of any one of clauses 36 to 72, further comprising using the sequence-activity dataset to train a machine learning model.

Clause 74. The method of any one of clauses 36 to 73, wherein the machine learning model comprises a protein language model.

Clause 75. A method of recording biological activity of protein variants in nucleic acid memory, the method comprising: (a) providing a plurality of nucleic acid constructs, each construct comprising: (i) a nucleic acid sequence encoding a variant of a protein of interest (POI); and (ii) a recording sequence comprising one or more editable nucleotides; (b) functionally coupling biological activity of each variant of the POI to activity of a programmable writer enzyme such that the writer enzyme modifies the recording sequence in proportion to or in response to the biological activity of the variant; (c) permitting the writer enzyme to introduce one or more sequence modifications into the recording sequence; (d) determining the sequence of the nucleic acid sequence encoding the variant of the POI and the sequence of the modified recording sequence; and (e) correlating, using at least one processor, the sequence of the variant of the POI with the sequence modifications in the recording sequence to generate a dataset associating protein sequence with recorded biological activity.

Clause 76. The method of clause 75, wherein the recording sequence is positioned on the same nucleic acid molecule as the nucleic acid sequence encoding the variant of the POI.

Clause 77. The method of clause 75 or 76, wherein the recording sequence comprises a plurality of editable nucleotides susceptible to base substitution by the writer enzyme.

Clause 78. The method of any one of clauses 75 to 77, wherein the programmable writer enzyme comprises a base editor.

Clause 79. The method of clause 78, wherein the base editor comprises a nucleic acid deaminase fused to a programmable DNA-binding protein.

Clause 80. The method of clause 79, wherein the programmable DNA-binding protein is directed to the recording sequence by a guide RNA.

Clause 81. The method of any one of clauses 75 to 80, wherein biological activity of the variant of the POI regulates transcription, assembly, or activation of the writer enzyme.

Clause 82. The method of any one of clauses 75 to 81, wherein the sequence modifications introduced into the recording sequence quantitatively reflect the biological activity of the variant.

Clause 83. The method of any one of clauses 75 to 82, wherein correlating the sequence of the variant with the sequence modifications comprises calculating an edit frequency or total number of edits within the recording sequence.

Clause 84. The method of clause 83, further comprising generating a quantitative activity value for each variant based on the edit frequency or total number of edits.

Clause 85. A plasmid library comprising a plurality of plasmids, each plasmid comprising: (a) a nucleic acid sequence encoding a variant of a protein of interest (POI); and (b) a recording barcode comprising a nucleic acid sequence positioned on the same plasmid as the nucleic acid sequence encoding the variant of the POI.

Clause 86. The plasmid library of clause 85, wherein the recording barcode is positioned adjacent to the nucleic acid sequence encoding the variant of the POI.

Clause 87. The plasmid library of clause 85 or 86, wherein the recording barcode comprises a plurality of editable nucleotides.

Clause 88. The plasmid library of clause 87, wherein the editable nucleotides comprise cytosine residues susceptible to C-to-T substitution.

Clause 89. The plasmid library of any one of clauses 85 to 88, wherein each plasmid comprises a unique recording barcode corresponding to a single variant of the POI.

Clause 90. The plasmid library of any one of clauses 85 to 89, wherein the plurality of plasmids comprises at least 10² distinct variants.

Clause 91. The plasmid library of any one of clauses 85 to 90, wherein each plasmid further comprises a nucleic acid sequence encoding a writer enzyme.

Clause 92. An isolated nucleic acid comprising: (a) a nucleic acid sequence encoding a variant of a protein of interest (POI); and (b) a recording sequence comprising one or more editable nucleotides positioned in cis with the nucleic acid sequence encoding the variant of the POI.

Clause 93. The isolated nucleic acid of clause 92, wherein the recording sequence is located within about 500 base pairs of the nucleic acid sequence encoding the variant.

Clause 94. The isolated nucleic acid of clause 92 or 93, wherein the recording sequence comprises about 10 to about 100 nucleotides.

Clause 95. The isolated nucleic acid of any one of clauses 92 to 94, further comprising a promoter operably linked to the nucleic acid sequence encoding the variant of the POI.

Clause 96. The isolated nucleic acid of any one of clauses 92 to 95, further comprising a sequence encoding a programmable writer enzyme.

Clause 97. A vector comprising the isolated nucleic acid of any one of clauses 92 to 96.

Clause 98. The vector of clause 97, wherein the vector is a plasmid.

Clause 99. A host cell comprising the vector of clause 97 or 98.

Clause 100. The host cell of clause 99, wherein the host cell is a prokaryotic cell.

Clause 101. The host cell of clause 99, wherein the host cell is a eukaryotic cell.

Clause 102. The host cell of any one of clauses 99 to 101, wherein biological activity of the variant of the POI modulates activity of a programmable writer enzyme in the host cell.

Clause 103. A kit for generating a sequence-activity dataset for variants of a protein of interest (POI), the kit comprising: (a) a plasmid library comprising a plurality of plasmids, each plasmid comprising: (i) a nucleic acid sequence encoding a variant of the POI; and (ii) a recording barcode comprising one or more editable nucleotides positioned on the same plasmid as the nucleic acid sequence encoding the variant of the POI; (b) a nucleic acid construct encoding a programmable writer enzyme capable of introducing sequence modifications into the recording barcode; (c) one or more reagents for expressing the plasmid library and the programmable writer enzyme in a host cell; and (d) instructions for: (i) coupling biological activity of variants of the POI to activity of the programmable writer enzyme to generate activity-dependent sequence modifications in the recording barcode; (ii) sequencing the nucleic acid sequence encoding the variants of the POI and the corresponding recording barcodes; and (iii) correlating variant sequences with barcode modifications to generate a sequence-activity dataset.

What is claimed is:

1. A system comprising:
(a) a plasmid library comprising a plurality of plasmids, each plasmid comprising:
(a) a nucleic acid sequence encoding a variant of a protein of interest (POI); and
(b) a recording barcode comprising a nucleic acid sequence positioned in cis with the nucleic acid sequence encoding the variant of the POI;
(b) a writer enzyme comprising a base-editing enzyme capable of introducing one or more sequence modifications in the recording barcode, wherein activity of the variant of the POI modulates expression or activity of the base-editing enzyme such that sequence modifications are introduced into the recording barcode in an activity-dependent manner; and
(c) a sequencing and data-processing subsystem comprising:
(a) a sequencing module configured to determine sequences of the nucleic acid sequence encoding the variant of the POI and the corresponding recording barcode; and
(b) at least one processor in communication with a memory storing instructions that, when executed, cause the processor to correlate sequence information of the variant of the POI with sequence modifications in the corresponding recording barcode to generate a sequence-activity dataset.

2. The system of claim 1, wherein the recording barcode comprises a synthetic nucleic acid sequence of about 10 to about 100 nucleotides.

3. The system of claim 1, wherein the recording barcode comprises a plurality of editable nucleotides susceptible to base substitution by the writer enzyme.

4. The system of claim 1, wherein the editable nucleotides comprise cytosine residues configured for C-to-T conversion or adenine residues configured for A-to-G conversion.

5. The system of claim 1, wherein the recording barcode is positioned adjacent to the nucleic acid encoding the variant of the POI.

6. The system of claim 1, wherein each plasmid comprises a unique recording barcode sequence corresponding to a single POI variant.

7. The system of claim 1, wherein the writer enzyme comprises a base editor fused to a programmable DNA-binding protein.

8. The system of claim 7, wherein the base editor comprises a deaminase.

9. The system of claim 7, wherein the programmable DNA-binding protein comprises a Cas protein or Cas nickase and wherein the programmable DNA-binding protein is guided to the recording barcode by a guide RNA.

10. The system of claim 1, wherein activity of the variant of the POI regulates transcription of the writer enzyme or activation of the writer enzyme.

11. The system of claim 1, wherein the POI is selected from the group consisting of a protease, a polymerase, a transcription factor, an aminoacyl-tRNA synthetase, a CRISPR-associated nuclease, an antibody or binding protein, and a protein-protein interaction partner.

12. The system of claim 1, wherein the POI is a CRISPR-associated nuclease and recognition of a target sequence modulates barcode editing.

13. The system of claim 1, wherein the sequencing module comprises a next-generation sequencing platform and wherein the processor calculates an edit frequency, an edit pattern, or a total number of edits within the recording barcode.

14. The system of claim 1, wherein the sequence-activity dataset comprises a plurality of POI variant sequences and corresponding activity values.

15. The system of claim 1, wherein the plasmid library comprises POI variants generated by site-saturation mutagenesis or error-prone PCR.

16. A method of generating a sequence-activity dataset for variants of a protein of interest (POI), the method comprising:
(d) constructing a plasmid library comprising a plurality of plasmids, each plasmid comprising:
(a) a nucleic acid sequence encoding a variant of the POI; and
(b) a recording barcode comprising a nucleic acid sequence positioned in cis with the nucleic acid sequence encoding the variant of the POI;
(e) expressing the plasmid library in a cell in the presence of a writer enzyme capable of introducing one or more sequence modifications in the recording barcode;
(f) coupling biological activity of each variant of the POI to expression or activity of the writer enzyme such that sequence modifications are introduced into the corresponding recording barcode in an activity-dependent manner;
(g) sequencing the nucleic acid sequence encoding the variant of the POI and the corresponding recording barcode; and
(h) using at least one processor in communication with a memory to correlate the nucleic acid sequence encoding the variant of the POI with sequence modifications in the corresponding recording barcode to generate a sequence-activity dataset.

17. A plasmid library comprising a plurality of plasmids, each plasmid comprising:
(i) a nucleic acid sequence encoding a variant of a protein of interest (POI); and
(j) a recording barcode comprising a nucleic acid sequence positioned on the same plasmid as the nucleic acid sequence encoding the variant of the POI.

18. The plasmid library of claim 17, wherein the recording barcode comprises a plurality of editable nucleotides.

19. The plasmid library of claim 17, wherein the plurality of plasmids comprises at least 10² distinct variants.

20. The plasmid library of claim 17, wherein each plasmid further comprises a nucleic acid sequence encoding a writer enzyme.


SEQUENCE LISTING (8 sequences)

SEQ ID NO: 1 - DNA, 4164 bp, synthetic construct
  source 1..4164: mol_type = other DNA
atgtcttctgaaaccggtccggttgcggttgacccgaccctgcgtcgtcgtatcgaaccgcacgaattcgaagttttcttcgacccgcgtgaactgcgtaaagaaacctgcctgctgtacgaaatcaactggggtggtcgtcactctatctggcgtcacacctctcagaacaccaacaaacacgttgaagttaacttcatcgaaaaattcaccaccgaacgttacttctgcccgaacacccgttgctctatcacctggttcctgtcttggtctccgtgcggtgaatgctctcgtgcgatcaccgaattcctgtctcgttacccgcacgttaccctgttcatctacatcgcgcgtctgtaccaccacgcggacccgcgtaaccgtcagggtctgcgtgacctgatctcttctggtgttaccatccagatcatgaccgaacaggaatctggttactgctggcgtaacttcgttaactactctccgtctaacgaagcgcactggccgcgttacccgcacctgtgggttcgtctgtacgttctggaactgtactgcatcatcctgggtctgccgccgtgcctgaacatcctgcgtcgtaaacagccgcagctgaccttcttcaccatcgcgctgcagtcttgccactaccagcgtctgccgccgcacatcctgtgggcgaccggtctgaaatccggtagcgaaacaccggggacttcagaatcggccaccccggagtctatgaatcaaaaattcattctggggttggctattggaatcacgtcggtggggtatggccttattgactatgaaaccaagaacatcattgacgcgggggtccgtttgttccccgaggccaatgtcgaaaataacgagggccgtcgtagtaaacgcggctcccgtcgtcttaaacgccgccgcatccaccgtcttgagcgcgtgaagaagttgctggaggattataacctgcttgatcagagccagatcccgcagtctaccaatccatatgctattcgtgtcaaagggctttctgaggcattatccaaggacgagctggtgattgcattacttcacattgctaaacgccgcggtattcataaaatcgatgtaatcgatagtaacgacgatgttggtaacgagctttcgacgaaggaacagttgaacaaaaactcgaaacttttgaaggataagttcgtttgtcagatccaacttgaacgtatgaatgaaggtcaagtccgtggagaaaagaatcgttttaaaactgccgatattatcaaggaaatcatccaattgcttaatgttcaaaaaaatttccaccaattagacgagaattttatcaacaaatacatcgaactggtggagatgcgtcgtgaatattttgaaggacctggaaagggcagtccttatgggtgggaaggggaccccaaggcctggtacgagaccttaatggggcattgtacttattttccagatgaattacgctcagtaaagtatgcgtatagcgcggacttgttcaacgcgttaaatgatttaaacaatctggttatccagcgcgacggcttatcaaagttggagtaccacgagaaatatcatattattgaaaatgtatttaagcagaagaaaaaaccgaccttaaaacaaattgctaatgaaattaacgtaaatccagaagatattaagggttatcgcatcacaaagagtggcaaacctcagttcacggagttcaaattataccatgatttaaagtccgttctttttgaccaatctattttggaaaacgaagatgtgcttgatcagattgctgaaatcttaacgatttaccaagataaagattcaattaaatcgaagttaactgagttggatattttactgaatgaagaagataaggagaacatcgctcaactgaccggatacaccggtactcatcgcttatccttaaagtgcattcgcttggtgttggaagaacaatggtactcctcgcgcaatcaaatggaaatcttcacacacctgaacatcaagcctaagaagatcaacctgacggcagctaataaaatcccgaaagccatgattgatgagttcatcttaagccctgtagtgaaacgtacattcggccaagctattaacttaatcaacaagatcatcgagaaatacggagtccccgaagatatcatcatcgagctggctcgcgagaacaattccaaggacaagcagaagtttattaatgagatgcagaaaaagaacgaaaacacccgcaagcgcattaacgaaattatcggtaaatacggaaaccaaaatgccaagcgtcttgtggagaagattcgcctgcatgatgaacaggagggtaaatgtctgtattcgttggagtctattcctttagaggatttgctgaataacccaaatcattatgaagtagatcacatcatcccgcgttccgtatcatttgacaacagttaccataacaaggtgcttgttaagcagtcggaagcaagcaagaaaagtaatcttacaccctaccaatacttcaattcaggcaagtcaaaactgagctataaccaatttaaacagcacatccttaacctttcaaagagccaggatcgtatttctaaaaagaagaaggaatatcttcttgaggagcgtgatattaataagttcgaggtccagaaagaattcattaatcgtaacttagtagacacgcgctacgctacccgtgaattaacaaattacttgaaagcgtacttctctgcaaacaatatgaacgttaaggtgaaaacgattaatgggtcctttacagattatttgcgcaaagtgtggaagtttaagaaggaacgcaaccacggatacaaacaccacgcggaagacgctttaatcatcgccaatgcagacttcttattcaaggagaacaagaaattaaaagcggttaattcagtgcttgaaaagcccgagatcgaatcgaagcagttagacatccaagttgactccgaggataactactctgaaatgtttattatcccgaagcaggtacaggacattaaggacttccgtaatttcaaatattcccaccgtgtcgataagaaaccaaaccgtcaattaattaacgatacattatacagcacccgtaagaaagacaactcaacttacatcgtgcaaaccatcaaggacatctatgcaaaggataacacaacactgaagaaacagttcgacaagagtccggagaaatttctgatgtaccaacacgacccgcgtaccttcgagaagttagaagtcatcatgaagcaatatgctaatgaaaaaaatcctcttgccaagtatcacgaagaaacgggagaatatctgaccaaatactccaagaaaaacaatggaccaatcgttaagagcctgaaatacattgggaataagttaggctcccacttggatgttacgcatcaatttaaaagttccactaaaaagcttgtgaagttaagtattaaaccataccgttttgatgtatatttgacagataagggttacaagtttattacaattagttacttggacgttttaaaaaaagacaactactactacatcccagagcagaaatatgataaattaaaattgggtaaagcgattgataagaatgctaagtttatcgccagtttctataaaaatgacctgatcaagctggacggggagatttacaaaatcatcggggttaactccgacacacgcaacatgatcgagcttgacttacccgatattcgttacaaggagtattgcgaacttaacaatattaagggggaacctcgtattaaaaagacaatcggcaaaaaagttaatagtattgaaaaactgacaaccgatgtattgggcaacgtctttacaaacacccagtacactaaacctcagcttctttttaaacgcggtaatagcggcggttcaatgacaaatctgtcggatatcatcgaaaaagaaaccggtaaacagctggttatccaagaaagtattttaatgcttcccgaagaagtcgaagaggtgattggcaacaaacccgaaagtgatattctcgtgcatactgcgtatgatgagtcgaccgatgaaaacgtcatgctgctgacctccgacgcgcccgagtataaaccgtgggctttggttatccaggatagcaacggtgaaaataagattaaaatgttataa

SEQ ID NO: 2 - DNA, 136 bp, synthetic construct
  source 1..136: mol_type = other DNA
ttgacagctagctcagtcctaggtataatactagtgtccacccgcaaaattaaaagttttagtactctggaaacagaatctactaaaacaaggcaaaatgccgtgtttatctcgtcaacttgttggcgagattttt

SEQ ID NO: 3 - DNA, 777 bp, synthetic construct
  source 1..777: mol_type = other DNA
atgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaatggccgcttttaattttaccctggaacgcagcgaacgacgaaaattatgcgttggcagcttaa

SEQ ID NO: 4 - DNA, 750 bp, synthetic construct
  source 1..750: mol_type = other DNA
atggcgattggccgcaagatgtttgagtgataacttagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataa

SEQ ID NO: 5 - DNA, 3714 bp, synthetic construct
  source 1..3714: mol_type = other DNA
atgagtgaggtcgaattttctcacgaatactggatgcgccacgctcttactttagccaagcgtgcgcgcgatgagcgcgaggtcccggtaggagctgtcctggtgttaaacaaccgtgtgatcggtgaaggttggaatcgtgctatcggtctgcatgatccaacggcccatgccgagatcatggcgcttcgccagggggggctggtcatgcaaaactaccgccttattgatgctacactgtatgtcactttcgagccttgtgtgatgtgtgcgggagccatgatccacagtcgtattggtcgtgtcgtttttggtgtacgtaacagtaaacgcggcgcagctggatccttaatgaacgtcttaaattatccaggcatgaatcatcgtgttgagattactgaaggcatcttagcggatgaatgtgccgcccttttgtgcgatttttaccgtatgcctcgccaagtgtttaacgcacagaaaaaggcgcaatcgtcgatcaattccggtagcgaaacaccggggacttcagaatcggccaccccggagtctatgaatcaaaaattcattctggggttggctattggaatcacgtcggtggggtatggccttattgactatgaaaccaagaacatcattgacgcgggggtccgtttgttccccgaggccaatgtcgaaaataacgagggccgtcgtagtaaacgcggctcccgtcgtcttaaacgccgccgcatccaccgtcttgagcgcgtgaagaagttgctggaggattataacctgcttgatcagagccagatcccgcagtctaccaatccatatgctattcgtgtcaaagggctttctgaggcattatccaaggacgagctggtgattgcattacttcacattgctaaacgccgcggtattcataaaatcgatgtaatcgatagtaacgacgatgttggtaacgagctttcgacgaaggaacagttgaacaaaaactcgaaacttttgaaggataagttcgtttgtcagatccaacttgaacgtatgaatgaaggtcaagtccgtggagaaaagaatcgttttaaaactgccgatattatcaaggaaatcatccaattgcttaatgttcaaaaaaatttccaccaattagacgagaattttatcaacaaatacatcgaactggtggagatgcgtcgtgaatattttgaaggacctggaaagggcagtccttatgggtgggaaggggaccccaaggcctggtacgagaccttaatggggcattgtacttattttccagatgaattacgctcagtaaagtatgcgtatagcgcggacttgttcaacgcgttaaatgatttaaacaatctggttatccagcgcgacggcttatcaaagttggagtaccacgagaaatatcatattattgaaaatgtatttaagcagaagaaaaaaccgaccttaaaacaaattgctaatgaaattaacgtaaatccagaagatattaagggttatcgcatcacaaagagtggcaaacctcagttcacggagttcaaattataccatgatttaaagtccgttctttttgaccaatctattttggaaaacgaagatgtgcttgatcagattgctgaaatcttaacgatttaccaagataaagattcaattaaatcgaagttaactgagttggatattttactgaatgaagaagataaggagaacatcgctcaactgaccggatacaccggtactcatcgcttatccttaaagtgcattcgcttggtgttggaagaacaatggtactcctcgcgcaatcaaatggaaatcttcacacacctgaacatcaagcctaagaagatcaacctgacggcagctaataaaatcccgaaagccatgattgatgagttcatcttaagccctgtagtgaaacgtacattcggccaagctattaacttaatcaacaagatcatcgagaaatacggagtccccgaagatatcatcatcgagctggctcgcgagaacaattccaaggacaagcagaagtttattaatgagatgcagaaaaagaacgaaaacacccgcaagcgcattaacgaaattatcggtaaatacggaaaccaaaatgccaagcgtcttgtggagaagattcgcctgcatgatgaacaggagggtaaatgtctgtattcgttggagtctattcctttagaggatttgctgaataacccaaatcattatgaagtagatcacatcatcccgcgttccgtatcatttgacaacagttaccataacaaggtgcttgttaagcagtcggaagcaagcaagaaaagtaatcttacaccctaccaatacttcaattcaggcaagtcaaaactgagctataaccaatttaaacagcacatccttaacctttcaaagagccaggatcgtatttctaaaaagaagaaggaatatcttcttgaggagcgtgatattaataagttcgaggtccagaaagaattcattaatcgtaacttagtagacacgcgctacgctacccgtgaattaacaaattacttgaaagcgtacttctctgcaaacaatatgaacgttaaggtgaaaacgattaatgggtcctttacagattatttgcgcaaagtgtggaagtttaagaaggaacgcaaccacggatacaaacaccacgcggaagacgctttaatcatcgccaatgcagacttcttattcaaggagaacaagaaattaaaagcggttaattcagtgcttgaaaagcccgagatcgaatcgaagcagttagacatccaagttgactccgaggataactactctgaaatgtttattatcccgaagcaggtacaggacattaaggacttccgtaatttcaaatattcccaccgtgtcgataagaaaccaaaccgtcaattaattaacgatacattatacagcacccgtaagaaagacaactcaacttacatcgtgcaaaccatcaaggacatctatgcaaaggataacacaacactgaagaaacagttcgacaagagtccggagaaatttctgatgtaccaacacgacccgcgtaccttcgagaagttagaagtcatcatgaagcaatatgctaatgaaaaaaatcctcttgccaagtatcacgaagaaacgggagaatatctgaccaaatactccaagaaaaacaatggaccaatcgttaagagcctgaaatacattgggaataagttaggctcccacttggatgttacgcatcaatttaaaagttccactaaaaagcttgtgaagttaagtattaaaccataccgttttgatgtatatttgacagataagggttacaagtttattacaattagttacttggacgttttaaaaaaagacaactactactacatcccagagcagaaatatgataaattaaaattgggtaaagcgattgataagaatgctaagtttatcgccagtttctataaaaatgacctgatcaagctggacggggagatttacaaaatcatcggggttaactccgacacacgcaacatgatcgagcttgacttacccgatattcgttacaaggagtattgcgaacttaacaatattaagggggaacctcgtattaaaaagacaatcggcaaaaaagttaatagtattgaaaaactgacaaccgatgtattgggcaacgtctttacaaacacccagtacactaaacctcagcttctttttaaacgcggtaattaa

SEQ ID NO: 6 - DNA, 3270 bp, synthetic construct
  source 1..3270: mol_type = other DNA
atgaaaatcgaagaaaatcaaaaattcattctggggttggctattggaatcacgtcggtggggtatggccttattgactatgaaaccaagaacatcattgacgcgggggtccgtttgttccccgaggccaatgtcgaaaataacgagggccgtcgtagtaaacgcggctcccgtcgtcttaaacgccgccgcatccaccgtcttgagcgcgtgaagaagttgctggaggattataacctgcttgatcagagccagatcccgcagtctaccaatccatatgctattcgtgtcaaagggctttctgaggcattatccaaggacgagctggtgattgcattacttcacattgctaaacgccgcggtattcataaaatcgatgtaatcgatagtaacgacgatgttggtaacgagctttcgacgaaggaacagttgaacaaaaactcgaaacttttgaaggataagttcgtttgtcagatccaacttgaacgtatgaatgaaggtcaagtccgtggagaaaagaatcgttttaaaactgccgatattatcaaggaaatcatccaattgcttaatgttcaaaaaaatttccaccaattagacgagaattttatcaacaaatacatcgaactggtggagatgcgtcgtgaatattttgaaggacctggaaagggcagtccttatgggtgggaaggggaccccaaggcctggtacgagaccttaatggggcattgtacttattttccagatgaattacgctcagtaaagtatgcgtatagcgcggacttgttcaacgcgttaaatgatttaaacaatctggttatccagcgcgacggcttatcaaagttggagtaccacgagaaatatcatattattgaaaatgtatttaagcagaagaaaaaaccgaccttaaaacaaattgctaatgaaattaacgtaaatccagaagatattaagggttatcgcatcacaaagagtggcaaacctcagttcacggagttcaaattataccatgatttaaagtccgttctttttgaccaatctattttggaaaacgaagatgtgcttgatcagattgctgaaatcttaacgatttaccaagataaagattcaattaaatcgaagttaactgagttggatattttactgaatgaagaagataaggagaacatcgctcaactgaccggatacaccggtactcatcgcttatccttaaagtgcattcgcttggtgttggaagaacaatggtactcctcgcgcaatcaaatggaaatcttcacacacctgaacatcaagcctaagaagatcaacctgacggcagctaataaaatcccgaaagccatgattgatgagttcatcttaagccctgtagtgaaacgtacattcggccaagctattaacttaatcaacaagatcatcgagaaatacggagtccccgaagatatcatcatcgagctggctcgcgagaacaattccaaggacaagcagaagtttattaatgagatgcagaaaaagaacgaaaacacccgcaagcgcattaacgaaattatcggtaaatacggaaaccaaaatgccaagcgtcttgtggagaagattcgcctgcatgatgaacaggagggtaaatgtctgtattcgttggagtctattcctttagaggatttgctgaataacccaaatcattatgaagtagatcacatcatcccgcgttccgtatcatttgacaacagttaccataacaaggtgcttgttaagcagtcggaagcaagcaagaaaagtaatcttacaccctaccaatacttcaattcaggcaagtcaaaactgagctataaccaatttaaacagcacatccttaacctttcaaagagccaggatcgtatttctaaaaagaagaaggaatatcttcttgaggagcgtgatattaataagttcgaggtccagaaagaattcattaatcgtaacttagtagacacgcgctacgctacccgtgaattaacaaattacttgaaagcgtacttctctgcaaacaatatgaacgttaaggtgaaaacgattaatgggtcctttacagattatttgcgcaaagtgtggaagtttaagaaggaacgcaaccacggatacaaacaccacgcggaagacgctttaatcatcgccaatgcagacttcttattcaaggagaacaagaaattaaaagcggttaattcagtgcttgaaaagcccgagatcgaatcgaagcagttagacatccaagttgactccgaggataactactctgaaatgtttattatcccgaagcaggtacaggacattaaggacttccgtaatttcaaatattcccaccgtgtcgataagaaaccaaaccgtcaattaattaacgatacattatacagcacccgtaagaaagacaactcaacttacatcgtgcaaaccatcaaggacatctatgcaaaggataacacaacactgaagaaacagttcgacaagagtccggagaaatttctgatgtaccaacacgacccgcgtaccttcgagaagttagaagtcatcatgaagcaatatgctaatgaaaaaaatcctcttgccaagtatcacgaagaaacgggagaatatctgaccaaatactccaagaaaaacaatggaccaatcgttaagagcctgaaatacattgggaataagttaggctcccacttggatgttacgcatcaatttaaaagttccactaaaaagcttgtgaagttaagtattaaaccataccgttttgatgtatatttgacagataagggttacaagtttattacaattagttacttggacgttttaaaaaaagacaactactactacatcccagagcagaaatatgataaattaaaattgggtaaagcgattgataagaatgctaagtttatcgccagtttctataaaaatgacctgatcaagctggacggggagatttacaaaatcatcggggttaactccgacacacgcaacatgatcgagcttgacttacccgatattcgttacaaggagtattgcgaacttaacaatattaagggggaacctcgtattaaaaagacaatcggcaaaaaagttaatagtattgaaaaactgacaaccgatgtattgggcaacgtctttacaaacacccagtacactaaacctcagcttctttttaaacgcggtaatgggtcgggcggcggcggctcgggcaagcgtacagccgacggaagtgagttcgagcctaaaaagaagcgtaaggtacatcaccatcaccatcattaa

SEQ ID NO: 7 - DNA, 1260 bp, synthetic construct
  source 1..1260: mol_type = other DNA
atggataagaagccgctggatgttctgatctctgcgaccggtctgtggatgtcccgtaccggcacgctgcacaagatcaagcactatgagatttctcgttctaaaatctacatcgaaatggcgtgtggtgaccatctggttgtgaacaactctcgttcttgtcgtcccgcacgtgcattccgttatcataaataccgtaaaacctgcaaacgttgtcgtgtttctgacgaagatatcaacaacttcctgacccgttctaccgaaggcaaaacctctgttaaagttaaagttgtttctgagccgaaagtgaaaaaagcgatgccgaaatctgtttctcgtgcgccgaaaccgctggaaaatccggtttctgcgaaagcgtctaccgacacctctcgttctgttccgtctccggcgaaatctaccccgaactctccggttccgacctctgcgccggcgccgtctctgacccgttctcagctggatcgtgttgaagcgctgctgtctccggaagataaaatctctctgaacatcgcgaaaccgttccgtgaactggaatctgaactggttacccgtcgtaaaaacgatttccagcgtctgtacaccaacgatcgtgaagactacctgggtaaactggaacgtgacatcaccaaattcttcgttgaccgtgatttcctggaaatcaaatctccgatcctgatcccggcggaatacgttgaacgtatgggtatcaacaacgataccgaactgtctaaacagatcttccgtgttgataaaaacctgtgcctgcgtccgatgctggcgccgaccctgtacaactatctgcgtaaactggatcgtatcctgccggacccgatcaaaatcttcgaagttggtccgtgctaccgtaaagaatctgacggtaaagaacacctggaagagttcaccatggtgaacttctgccagatgggttctggttgcacccgtgagaacctggaatctctgatcaaagaatttctggactacctggaaatcgacttcgaaatcgttggtgactcctgcatggtgtacggtgataccctggacatcatgcacggtgacctggaactgtcttctgcggttgttggtccggttccgctggatcgtgaatggggtatcgacaaaccgtggatcggtgcgggtttcggtctggaacgtctgctgaaagttatgcacggtttcaaaaacatcaaacgtgcgtctcgttctgaatcttactacaacggtatctctaccaacctgtaa

SEQ ID NO: 8 - DNA, 252 bp, synthetic construct
  source 1..252: mol_type = other DNA
tgtgcttctcaaatgcctgaggccagtttgctcaggctctccccgtggaggtaataattgacgatatgatcagtgcacggctaactaagcggcctgctgactttctcgccgatcaaaaggcattttgctattaagggattgacgagggcgtatctgcgcagtaagatgcgccccgcattcggaaacgtgatcatgtagatcgaatggactctaaatccgttcagtggggttagattccccacgtttccgcca